(19) United States
(12) Patent Application Publication
(10) Pub. No.: US 2025/0380697 A1
(43) Pub. Date: Dec. 18, 2025

(54) ISOXAZOLINE DERIVATIVE COMPOUND AND INSECTICIDAL COMPOSITION COMPRISING SAME

(71) Applicants: KYUNG NONG CORPORATION, Seoul (KR); KOREA RESEARCH INSTITUTE OF CHEMICAL TECHNOLOGY, Daejeon (KR)

(72) Inventors: Young Bin KWON, Gyeongju-si (KR); Shizuka Mei MAEZONO, Gyeongju-si (KR); Bowon KWON, Gyeongju-si (KR); Won Seok CHOI, Gyeongju-si (KR); Jin Won SEO, Gyeongju-si (KR); Won Jin KANG, Gyeongju-si (KR); Seong Jun PARK, Daejeon (KR); Hwan Jung LIM, Daejeon (KR)

(73) Assignees: KYUNG NONG CORPORATION, Seoul (KR); KOREA RESEARCH INSTITUTE OF CHEMICAL TECHNOLOGY, Daejeon (KR)

(21) Appl. No.: 18/879,376

(22) PCT Filed: Jun. 28, 2023
(86) PCT No.: PCT/KR2023/009056

§ 371 Date: Dec. 27, 2024

(30) Foreign Application Priority Data
Jun. 30, 2022 (KR) 10-2022-0080693

(51) Int. Cl. A01N 43/56; A01N 43/08; A01N 43/10; A01N 43/40; A01N 43/78; A01N 43/80; A01P 7/04; C07D 261/04; C07D 261/18; C07D 413/04; C07D 413/12; C07D 417/12

(52) U.S. Cl. CPC A01N 43/56; A01N 43/08; A01N 43/10; A01N 43/40; A01N 43/78; A01N 43/80; A01P 7/04; C07D 261/04; C07D 261/18; C07D 413/04; C07D 413/12; C07D 417/12

(57) ABSTRACT

A novel isoxazoline derivative compound represented by Formula 1 below, a stereoisomer thereof, a hydrate thereof, or a salt thereof is disclosed. And an insecticidal composition containing the novel isoxazoline derivative compound and uses thereof are disclosed. The insecticidal composition can exhibit excellent insecticidal effects against various types of pests.

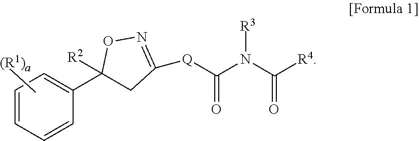

### TECHNICAL FIELD

The present invention relates to a novel isoxazoline derivative compound and a pesticide composition containing the same.

### BACKGROUND ART

Pests can be generally divided into sucking pests, such as aphids and shield bugs, and lepidopteran pests, which are foliar-feeding pests that eat leaves. These pests cause significant damage by extracting nutrients from the roots, stems, or leaves of trees and crops, or by gnawing on these parts themselves. Therefore, pest control is important for the management of trees and crops.

Various types of pesticides have been developed and used to control these pests. However, despite the development of various types of pesticides, there are limits to effective control of pests as resistance to pesticides develops. Accordingly, although the use of highly toxic and high-concentration pesticides is being considered, but this can cause not only serious contamination of the soil, but also secondary damage to humans or livestock that eat the crops due to pesticides remaining in the crops.

Therefore, there is a need for new pesticide substances that are safer for humans and livestock while exhibiting excellent control effects against pests at relatively low concentrations.

### PRIOR ART DOCUMENT

### Patent Document

(Patent Document 1) Korean Patent No. 10-2267724

### DISCLOSURE OF INVENTION

### Technical Problem

An object of the present invention is to provide a novel isoxazoline derivative compound and a pesticide composition including the same, which thus has excellent control effects against various pests.

Another object of the present invention is to provide a method for controlling pests using the isoxazoline derivative compound.

### Solution to Problem

In order to solve the above problems, the present invention provides a compound represented by the following Formula (1), a stereoisomer thereof, a hydrate thereof, or a salt thereof:

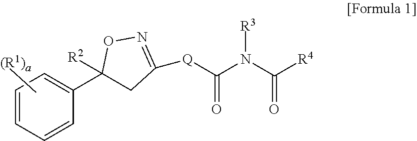

In Formula 1,
R¹ is each independently hydrogen, halogen, cyano (CN), C₁₋₅ alkyl, or C₁₋₅ haloalkyl,
R² is hydrogen, halogen, C₁₋₅ alkyl, or C₁₋₅ haloalkyl,
R³ is hydrogen, C₁₋₅ alkyl, C₃₋₁₀ cycloalkyl, —C(═O)—C₃₋₁₀ cycloalkyl; -C₁₋₅ alkylene-O—C(═O)H substituted with one or more selected from the group consisting of C₁₋₅ alkyl, C₁₋₅ haloalkyl, and C₃₋₁₀ cycloalkyl; or C₁₋₁₀ alkyl substituted with one or more selected from the group consisting of halogen, C₁₋₅ alkyl, C₁₋₅ haloalkyl, and C₃₋₁₀ cycloalkyl,
R⁴ is C₁₋₅ alkyl, C₁₋₅ haloalkyl, C₃₋₁₀ cycloalkyl, C₅₋₁₂ spiroalkyl, 3- to 10-membered heterocycloalkyl, 3- to 10-membered heterocycloalkylene-C(═O)—O—C₁₋₅ alkyl, C₂₋₁₀ alkenyl, C₆₋₂₀ aryl, 3- to 10-membered heteroaryl; C₁₋₅ alkyl substituted with C₃₋₁₀ cycloalkyl; C₃₋₁₀ cycloalkyl substituted with one or more selected from the group consisting of cyano (CN), halogen, C₁₋₅ alkyl, C₁₋₅ haloalkyl, and C₃₋₁₀ cycloalkyl; 3- to 10-membered heterocycloalkyl substituted with one or more selected from the group consisting of halogen, C₁₋₅ alkyl, C₁₋₅ haloalkyl, and C₃₋₁₀ cycloalkyl; C₂₋₁₀ alkenyl substituted with one or more selected from the group consisting of C₁₋₅ alkyl and C₆₋₂₀ aryl; C₆₋₂₀ aryl substituted with one or more selected from the group consisting of cyano (CN), halogen, C₁₋₅ alkyl, and C₁₋₅ haloalkyl; or 3- to 10-membered heteroaryl substituted with one or more selected from the group consisting of halogen, C₁₋₅ alkyl, C₁₋₅ haloalkyl, and C₃₋₁₀ cycloalkyl,
Q is C₆₋₂₀ arylene, 3- to 10-membered heteroarylene, or C₆₋₂₀ arylene substituted with one or more selected from the group consisting of halogen, and C₁₋₅ alkyl,
a is an integer of 1 to 5, and
the heterocycloalkyl, the heteroaryl, the heterocycloalkylene and the heteroarylene each comprise at least one heteroatom selected from the group consisting of N, O, and S.

The present invention also provides a pesticide composition, which includes one or more compounds selected from the group consisting of the compound, a stereoisomer thereof, a hydrate thereof, and a salt thereof as active ingredients.

The present invention also provides a method for controlling pests, which includes treating crops or their habitats with the pesticide composition.

### Advantageous Effects of Invention

The pesticide composition including the novel isoxazoline derivative compound according to the present invention can exhibit excellent control (pesticidal) effect against various pests, particularly those of the order Thrips (e.g., Frankliniella occidentalis and Thrips tabaci Lindeman) or from the order Lepidoptera (e.g., Maruca vitrta, Spodoptera litura, Lymantria dispar, Helicoverpa armigera, and Plutella xylostella).

### BEST MODE FOR CARRYING OUT THE INVENTION

Hereinafter, the present invention will be described in detail. The present invention herein is not limited to the disclosures given below, but it may be modified into various forms as long as the gist of the invention is not changed.

In the present specification, the term “comprising” is intended to specify a particular characteristic, region, step, process, element, and/or component. It does not exclude the presence or addition of any other characteristic, region, step, process, element, and/or component, unless specifically stated to the contrary.

In the present specification, the term “substituted” includes not only the case where each hydrogen or functional group is substituted with one or more substituents, but also the case where the substituent is again substituted with one or more is substituents.

In the present specification, the expression of “*” or “ [symbol] ” indicates the position (site) where the functional group is bound.

[symbol]

### Isoxazoline Derivative Compound

The present invention provides a novel isoxazoline derivative compound. Specifically, one embodiment of the present invention provides a compound represented by Formula 1 below, a stereoisomer thereof, a hydrate thereof, or a salt thereof:

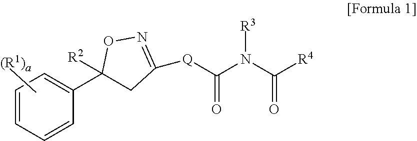

In Formula 1,
R¹ is each independently hydrogen, halogen, cyano (CN), C₁₋₅ alkyl, or C₁₋₅ haloalkyl,
R² is hydrogen, halogen, C₁₋₅ alkyl, or C₁₋₅ haloalkyl,
R³ is hydrogen, C₁₋₅ alkyl, C₃₋₁₀ cycloalkyl, —C(═O)—C₃₋₁₀ cycloalkyl; -C₁₋₅ alkylene-O—C(═O)H substituted with one or more selected from the group consisting of C₁₋₅ alkyl, C₁₋₅ haloalkyl, and C₃₋₁₀ cycloalkyl; or C₁₋₁₀ alkyl substituted with one or more selected from the group consisting of halogen, C₁₋₅ alkyl, C₁₋₅ haloalkyl, and C₃₋₁₀ cycloalkyl,
R⁴ is C₁₋₅ alkyl, C₁₋₅ haloalkyl, C₃₋₁₀ cycloalkyl, C₅₋₁₂ spiroalkyl, 3- to 10-membered heterocycloalkyl, 3- to 10-membered heterocycloalkylene-C(═O)—O—C₁₋₅ alkyl, C₂₋₁₀ alkenyl, C₆₋₂₀ aryl, 3- to 10-membered heteroaryl; C₁₋₅ alkyl substituted with C₃₋₁₀ cycloalkyl; C₃₋₁₀ cycloalkyl substituted with one or more selected from the group consisting of cyano (CN), halogen, C₁₋₅ alkyl, C₁₋₅ haloalkyl, and C₃₋₁₀ cycloalkyl; 3 to 10-membered heterocycloalkyl substituted with one or more selected from the group consisting of halogen, C₁₋₅ alkyl, C₁₋₅ haloalkyl, and C₃₋₁₀ cycloalkyl; C₂₋₁₀ alkenyl substituted with one or more selected from the group consisting of C₁₋₅ alkyl and C₆₋₂₀ aryl; C₆₋₂₀ aryl substituted with one or more selected from the group consisting of cyano (CN), halogen, C₁₋₅ alkyl, and C₁₋₅ haloalkyl; or 3- to 10-membered heteroaryl substituted with one or more selected from the group consisting of halogen, C₁₋₅ alkyl, C₁₋₅ haloalkyl, and C₃₋₁₀ cycloalkyl,
Q is C₆₋₂₀ arylene, 3- to 10-membered heteroarylene, or C₆₋₂₀ arylene substituted with one or more selected from the group consisting of halogen, and C₁₋₅ alkyl,
a is an integer of 1 to 5, and
the heterocycloalkyl, the heteroaryl, the heterocycloalkylene and the heteroarylene each comprise at least one heteroatom selected from the group consisting of N, O, and S. As used herein, the term “alkyl” may refer to a functional group in a linear-chain or branched-chain that does not include any double or triple bonds. Specifically, examples of alkyl may include methyl, ethyl, propyl, isopropyl, n-butyl, isobutyl, sec-butyl, t-butyl, pentyl, etc., but are not limited thereto.

As used herein, the term “haloalkyl” may refer to a functional group in which one to five (specifically, one to three, or one to two) halogens are substituted for alkyl. Specifically, examples of haloalkyl may include trifluoromethyl, trichloromethyl, difluoroethyl, dichloroethyl, etc., but are not limited thereto.

As used herein, the term “cycloalkyl” may refer to a cyclic functional group that does not include any double or triple bonds. Specifically, examples of cycloalkyl may include cyclopropane, cyclobutane, cyclopentane, cyclohexane, etc., but are not limited thereto.

As used herein, the term “spiroalkyl” may refer to a functional group in a form where two rings that do not include any double or triple bonds are connected by sharing one atom.

As used herein, the term “heterocycloalkyl” may refer to a cyclic functional group which has one or more heteroatoms without including any double or triple bonds. Specifically, examples of heterocycloalkyl may include azetidine, pyrrolidine, tetrahydrofuran, tetrahydrothiophene, tetrahydropyran, piperidine, perhydroazepine, oxacycloheptane, etc., but are not limited thereto.

As used herein, the term “alkenyl” may refer to a functional group in a linear-chain or branched-chain having that has one or more double bonds. Specifically, examples of alkenyl may include vinyl, butenyl, pentenyl, hexenyl, etc., but are not limited thereto.

As used herein, the term “aryl” may refer to a cyclic functional group including one or more double bonds. Specifically, examples of aryl may include phenyl, naphthyl, biphenyl, anthryl, phenanthryl, etc., but are not limited thereto.

As used herein, the term “heteroaryl” may refer to a cyclic functional group which has one or more heteroatoms while including one or more double bonds. Specifically, examples of heteroaryl may include pyrrole, thiophene, furan, pyrazole, isoxazole, thiazole, pyridine, quinoline, etc., but are not limited thereto.

As used herein, the term “heterocycloalkylene” may refer to a cyclic divalent functional group which has one or more heteroatoms while not including any double or triple bonds.

As used herein, the term “arylene” may refer to a cyclic divalent functional group including one or more double bonds. Specifically, examples of arylene may include phenylene, naphthalene, biphenylene, anthracene, etc., but are not limited thereto.

As used herein, the term “heteroarylene” may refer to a cyclic divalent functional group which includes one or more double bonds and has one or more heteroatoms.

As used herein, the term “hetero atom” may refer to an atom other than carbon or hydrogen. Specifically, hetero atoms may include N, O, S, etc., but are not limited thereto.

According to an embodiment of the present invention, in Formula 1 above, the Q may have a structure represented by

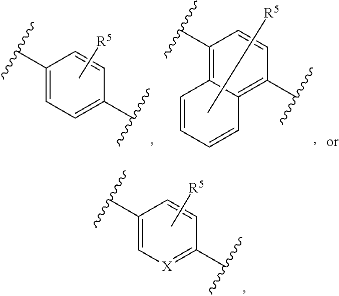

in which R⁵ may be hydrogen, halogen, or C₁₋₅ alkyl, and X may be N, O, or S.

According to an embodiment of the present invention, Formula 1 above may be specified as Formulas 1A to 1E below, but is not limited thereto. Specifically, an embodiment of the present invention may provide a compound represented by any one of the following Formulas 1A to 1E, a stereoisomer thereof, a hydrate thereof, or a salt thereof:

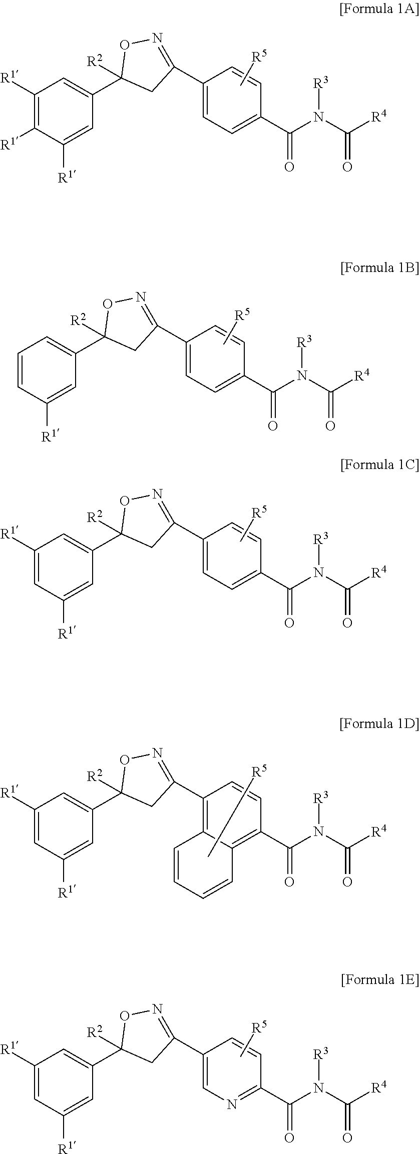

In Formulas TA to TB,

R^{1′} is each independently halogen, cyano (CN), C₁₋₅ alkyl, or C₁₋₅ haloalkyl, the definitions of R² to R⁴ are the same as defined above, and

R⁵ is hydrogen, halogen, or C₁₋₅ alkyl.

More specifically, according to an embodiment of the present invention, in Formulas 1A to 1E above, R^{1′} may each be independently cyano (CN), chlorine (Cl), fluorine (F), or C₁₋₃ haloalkyl (e.g., trifluoromethyl (CF₃)), and R² may be C₁₋₃ haloalkyl (e.g., trifluoromethyl (CF₃)).

According to an embodiment of the present invention, in Formula 1 above (specifically Formulas 1A to 1E), the R³ may specifically be hydrogen, methyl, ethyl,

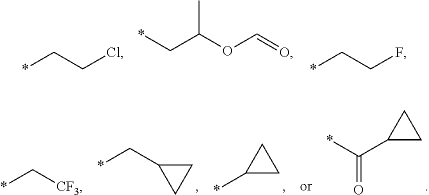

Additionally, according to an embodiment of the present invention, in Formula 1 (specifically Formulas 1A to 1E) above, the R⁴ may specifically be C₃₋₆ cycloalkyl; C₅₋₈ spiroalkyl; 3- to 6-membered heterocycloalkyl; 3- to 6-membered heterocycloalkylene-C(═O)—O—C₁₋₃ alkyl; C₂₋₅ alkenyl; C₆₋₁₀ aryl, 3- to 6-membered heteroaryl; C₁₋₃ alkyl substituted with C₃₋₆ cycloalkyl; C₃₋₆ cycloalkyl substituted with one or more selected from the group consisting of cyano (CN), halogen, and C₁₋₃ alkyl; C₂₋₅ alkenyl substituted with C₆₋₁₀ aryl; C₆₋₁₀ aryl substituted with cyano (CN); or 3- to 6-membered heteroaryl substituted with one or more selected from the group consisting of halogen, C₁₋₃ alkyl, C₁₋₃ haloalkyl, and C₃₋₆ cycloalkyl.

More specifically, in Formula 1 (specifically Formulas 1A to 1E) above, the R⁴ may be a substituent represented by

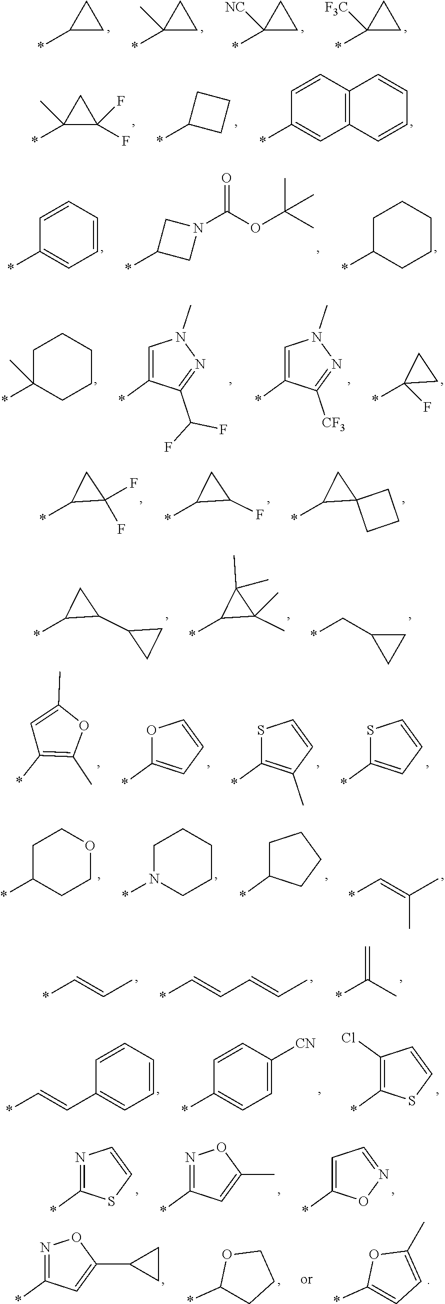

According to an embodiment of the present invention, Formula 1 above, by including, in the molecule, a structure in which an aromatic or heteroaromatic moiety and two carbonyls (C=0) are connected by an amine, an excellent pesticidal effect on pests may be exhibited. In particular, R⁴(e.g., cyclopropane) and isoxazoline moiety, which are bonded closely to the carbonyl (C=0), thereby exhibiting an excellent pesticidal effect on those of the order Thrips (e.g., western flower thrips, Thrips tabaci Lindeman) or from the order Lepidoptera (e.g., maruca pod borer, tobacco cutworm, gypsy moth, cotton bollworm, and diamondback moth).

According to an embodiment of the present invention, Formula 1 above may be is specified as having the following structures, but is not limited thereto. Specifically, an embodiment of the present invention may provide a compound represented by any one of Compounds 1001 to 1085 below, a stereoisomer thereof, a hydrate thereof, or a salt thereof.

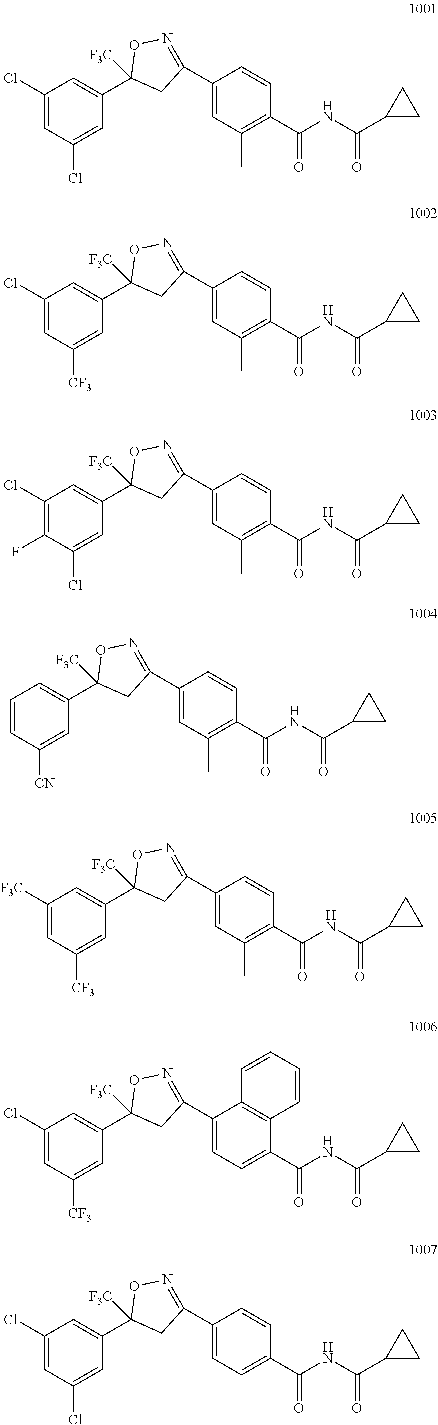

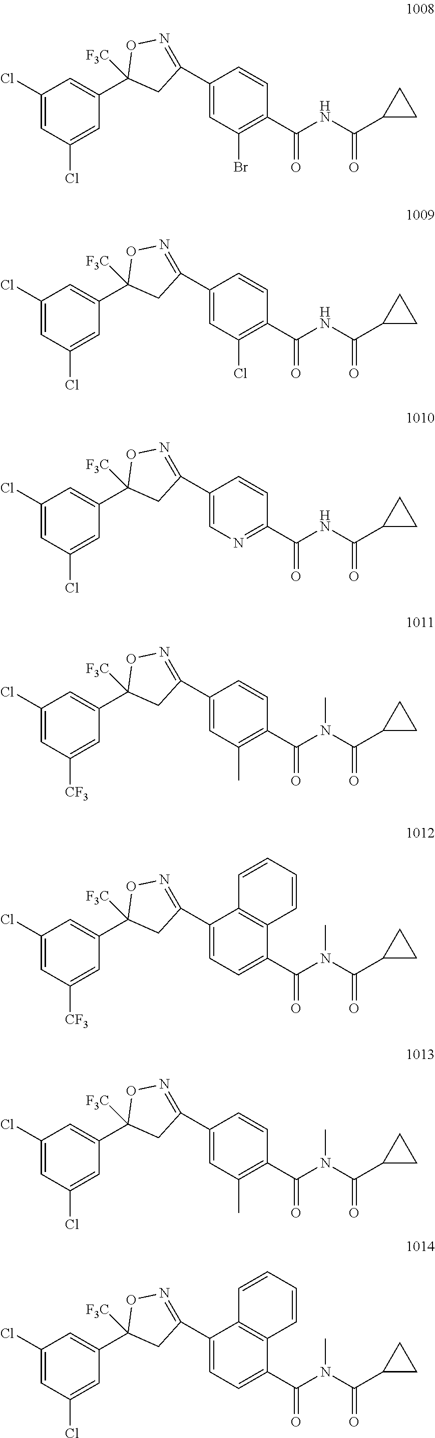

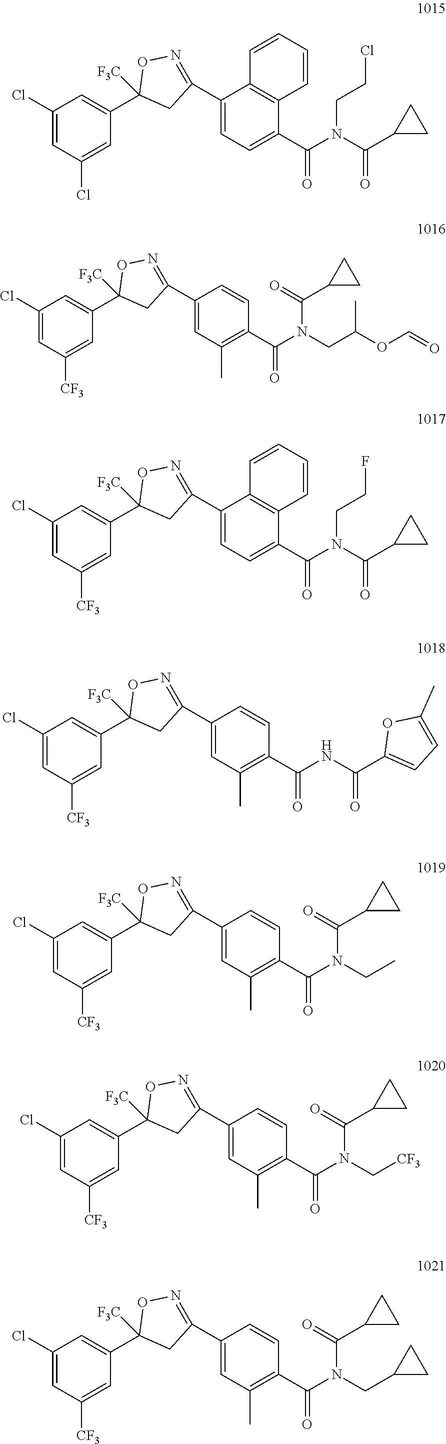

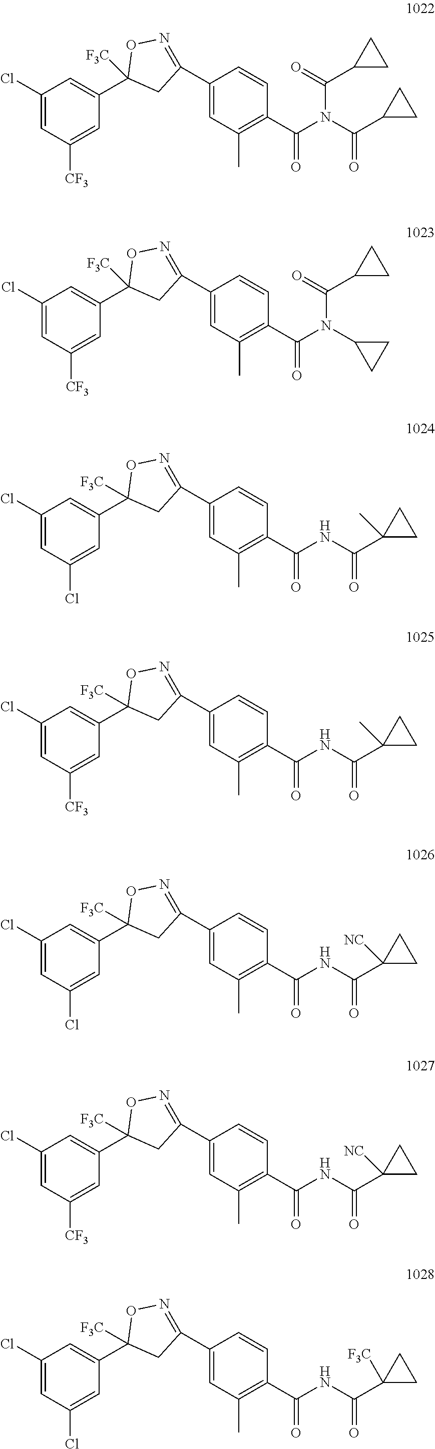

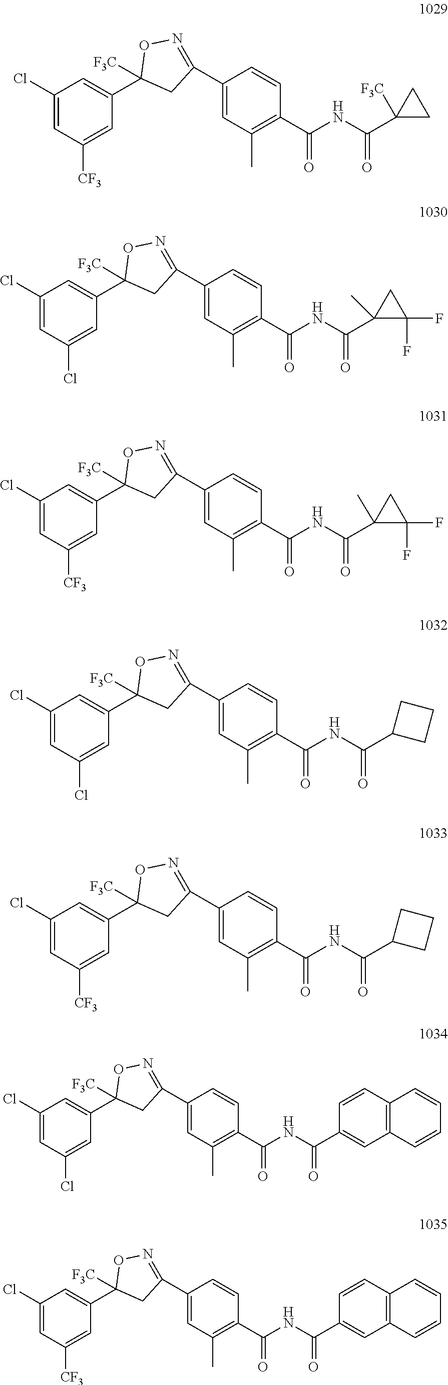

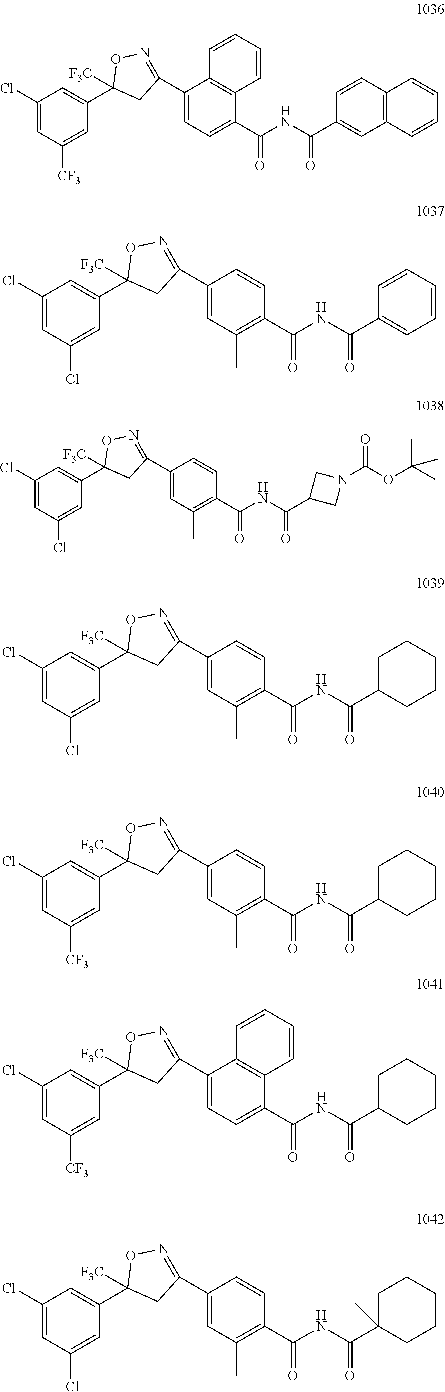

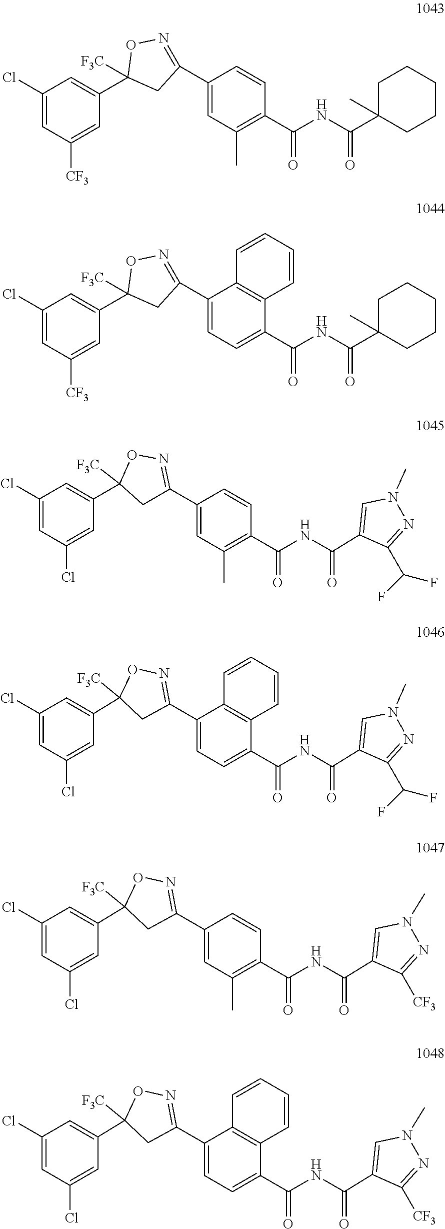 [SMILES: Cc1cc(C2=NOC(c3cc(Cl)cc(C(F)(F)F)c3)(C(F)(F)F)C2)ccc1C(=O)NC(=O)C1(C)CCCCC1 ; CC1(C(=O)NC(=O)c2ccc(C3=NOC(c4cc(Cl)cc(C(F)(F)F)c4)(C(F)(F)F)C3)c3ccccc23)CCCCC1 ; Cc1cc(C2=NOC(c3cc(Cl)cc(Cl)c3)(C(F)(F)F)C2)ccc1C(=O)NC(=O)c1cn(C)nc1C(F)F ; Cn1cc(C(=O)NC(=O)c2ccc(C3=NOC(c4cc(Cl)cc(Cl)c4)(C(F)(F)F)C3)c3ccccc23)c(C(F)F)n1 ; Cc1cc(C2=NOC(c3cc(Cl)cc(Cl)c3)(C(F)(F)F)C2)ccc1C(=O)NC(=O)c1cn(C)nc1C(F)(F)F ; Cn1cc(C(=O)NC(=O)c2ccc(C3=NOC(c4cc(Cl)cc(Cl)c4)(C(F)(F)F)C3)c3ccccc23)c(C(F)(F)F)n1]

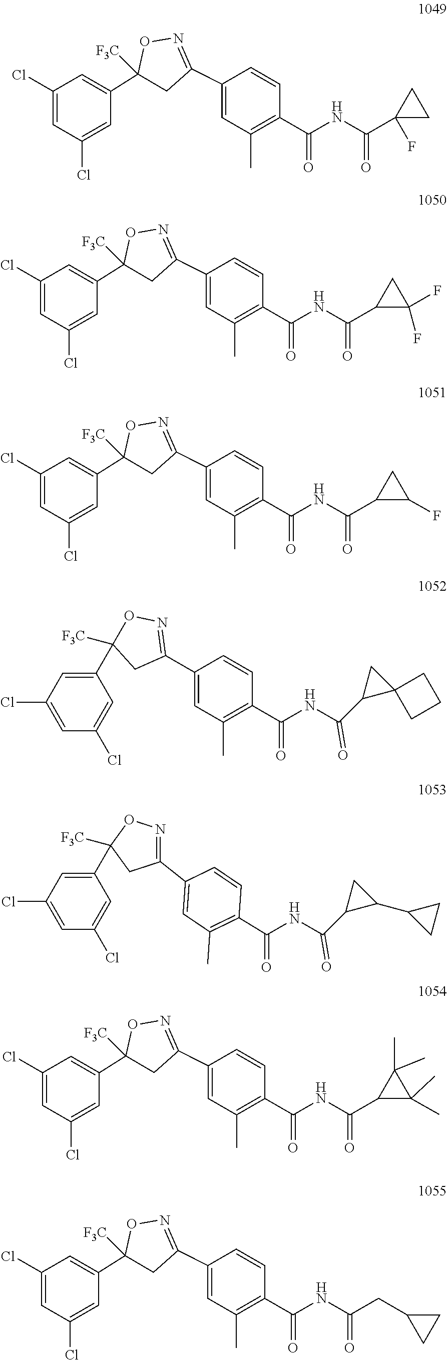

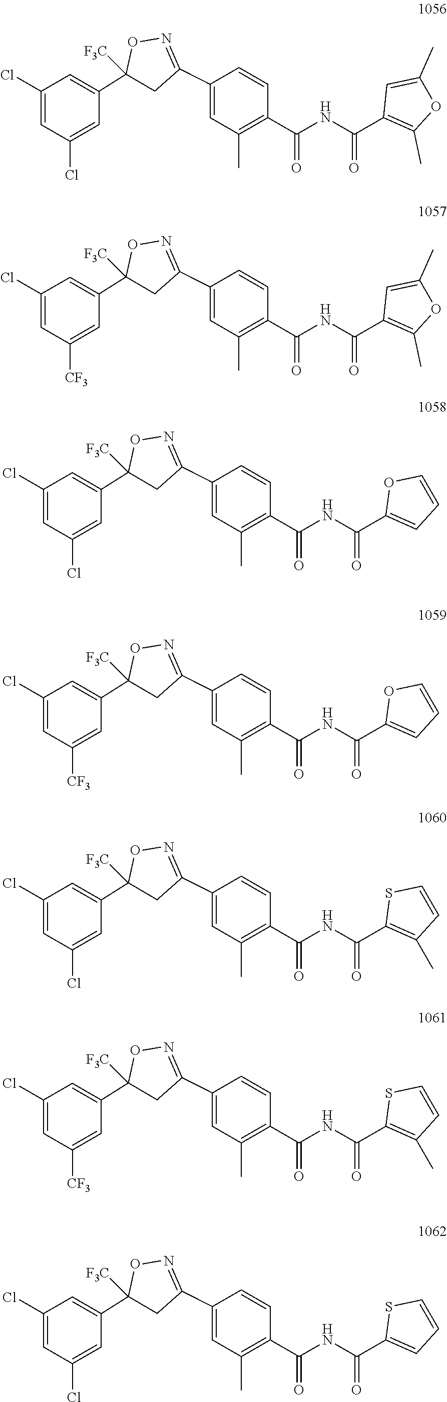

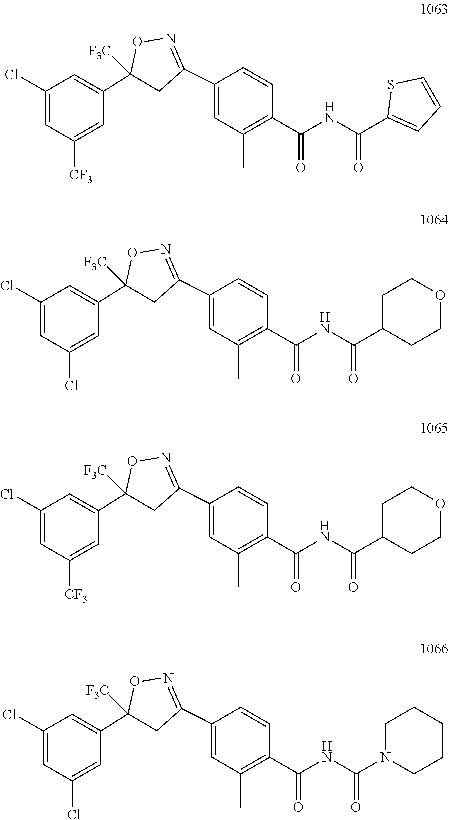 [SMILES: Cc1cc(C2=NOC(c3cc(Cl)cc(C(F)(F)F)c3)(C(F)(F)F)C2)ccc1C(=O)NC(=O)c1cccs1 ; Cc1cc(C2=NOC(c3cc(Cl)cc(Cl)c3)(C(F)(F)F)C2)ccc1C(=O)NC(=O)C1CCOCC1 ; Cc1cc(C2=NOC(c3cc(Cl)cc(C(F)(F)F)c3)(C(F)(F)F)C2)ccc1C(=O)NC(=O)C1CCOCC1 ; Cc1cc(C2=NOC(c3cc(Cl)cc(Cl)c3)(C(F)(F)F)C2)ccc1C(=O)NC(=O)N1CCCCC1]

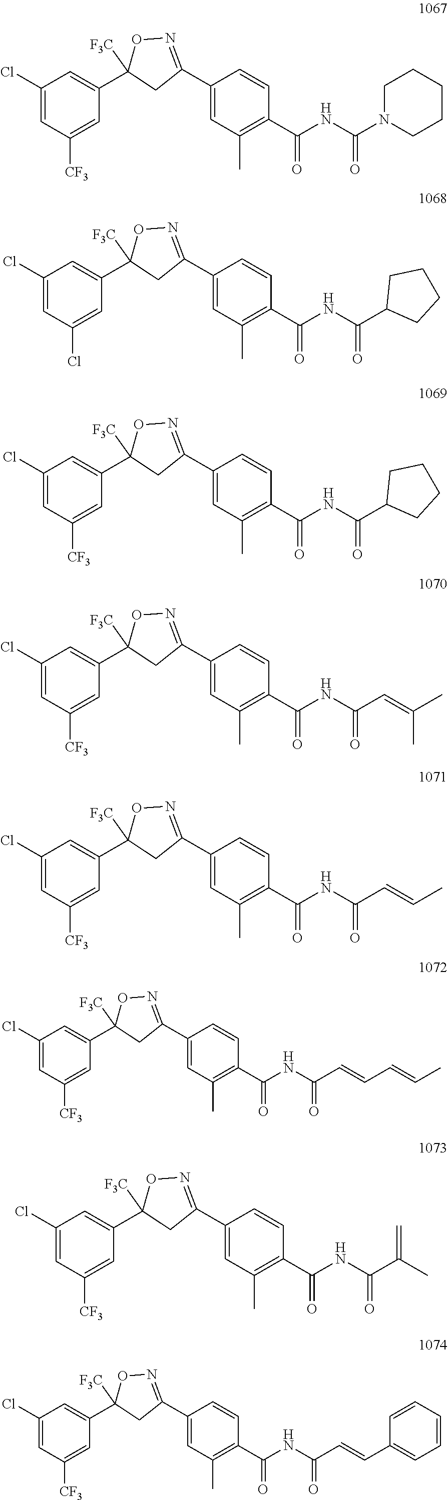

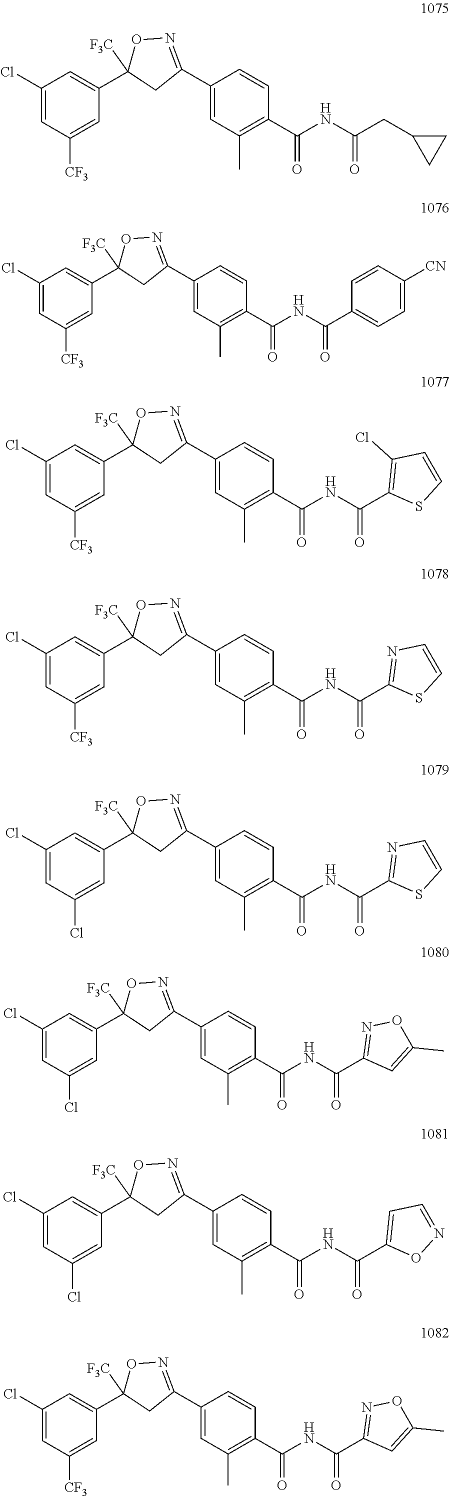

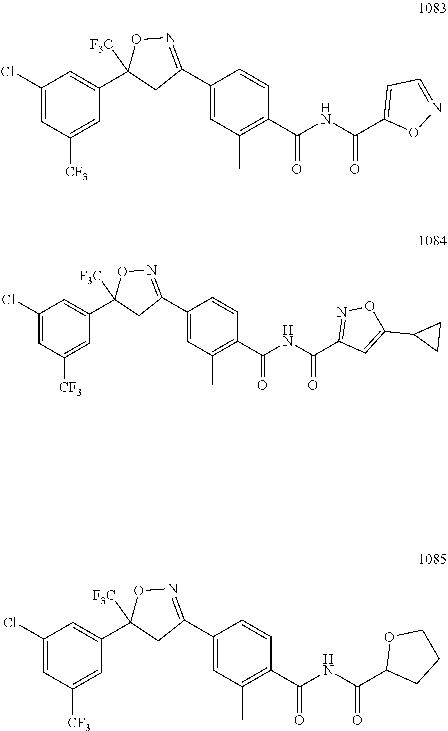 [SMILES: Cc1cc(C2=NOC(c3cc(Cl)cc(C(F)(F)F)c3)(C(F)(F)F)C2)ccc1C(=O)NC(=O)c1ccno1 ; Cc1cc(C2=NOC(c3cc(Cl)cc(C(F)(F)F)c3)(C(F)(F)F)C2)ccc1C(=O)NC(=O)c1cc(C2CC2)on1 ; Cc1cc(C2=NOC(c3cc(Cl)cc(C(F)(F)F)c3)(C(F)(F)F)C2)ccc1C(=O)NC(=O)C1CCCO1]

According to an embodiment of the present invention, the salt of the compound represented by Formula 1 above may be a salt of an agricultural or horticulturally acceptable inorganic acid or organic acid. Specifically, examples of the salt may include salts of inorganic acids such as bromic acid, hydrochloric acid, and sulfuric acid; salts of organic acids such as acetic acid, butyric acid, lactic acid, maleic acid, malonic acid, oxalic acid, propionic acid, and tartaric acid; salts of alkali metals such as lithium, sodium, and potassium; salts of alkaline earth metals such as calcium and magnesium; salts of transition metals such as iron and copper; salts of organic bases such as ammonia, triethylamine, tributylamine, pyridine, and hydrazine, but are not limited thereto. These salts may be prepared by commonly known methods.

According to an embodiment of the present invention, a hydrate of the compound represented by Formula 1 above may include stoichiometric or non-stoichiometric water bound by a non-covalent intermolecular force, a compound represented by Formula 1 above, a stereoisomer thereof, or a salt thereof. Such a hydrate may be prepared by commonly known methods.

### Pesticide Composition

The present invention provides a pesticide composition, which includes, as an active ingredient(s), one or more compounds selected from the group consisting of the compound represented by Formula 1 above, a stereoisomer thereof, a hydrate thereof, and a salt thereof.

According to an embodiment of the present invention, the pesticide composition may further include additives commonly known in the pesticide field. Specifically, examples of the additives may include surfactants, solid diluents, liquid diluents, dispersants, wetting agents, adhesives, solvents, or other active ingredients showing pesticidal activity, but are not limited thereto.

According to an embodiment of the present invention, the pesticide composition may be a spray composition, a bait composition, or a trap composition.

According to an embodiment of the present invention, the pesticide composition may be formulated in the form of a spray liquid, concentrate, wettable powder, fluid, granule, aerosol, smoking, sheet, etc.

The pesticide composition according to an embodiment of the present invention may exhibit pesticidal activity against pests or parasites. Specifically, the pesticide composition may exhibit pesticidal activity against cockroaches, ants, termites, mosquitoes, black flies, stable flies, deer flies, horse botflies, wasps, yellow jackets, bumblebees, ticks, spiders, moths, etc.

In particular, the pesticide composition according to an embodiment of the present invention may exhibit excellent pesticidal activity against the pests of the order Thrips and/or pests of the order Lepidoptera. Specifically, the pesticide composition according to the present invention may be used for controlling pests of the order Thrips or order of Lepidoptera.

For example, the pesticide composition may be for the control of Frankliniella occidentalis, Frankliniella tenuicornis, Frankliniella intonsa, Frankliniella lilivora, Thrips palmi Karny, Thrips tabaci Lindeman, Phaedon brassicae, Myzus persicae, Riptortus clavatus, Lymantria dispar, Helicoverpa armigera, Manulea degenerella, Rhopobota naevana, Grapholita molesta, Plutella xylostella, Spodoptera litura, Spodoptera exigua, Spodoptera frugiperda, Maruca vitrta. In particular, the pesticide composition according to an embodiment of the present invention may exhibit a remarkably excellent pesticidal activity against the pests of Frankliniella occidentalis, Thrips tabaci Lindeman, Spodoptera litura, Lymantria dispar, Helicoverpa armigera, Plutella xylostella, or Maruca vitrta.

The pesticide composition according to an embodiment of the present invention, when it is for the control of the above-described pests, may include one or more compounds selected from the group consisting of the compound represented by Formula 1 above, a stereoisomer thereof, a hydrate thereof, and a salt thereof, which are an active ingredient(s), in the amount of 0.0001 wt % to 95 wt %, 0.001 wt % to 90 wt %, 0.01 wt % to 85 wt %, 0.1 wt % to 70 wt %, 1 wt % to 60 wt %, 3 wt % to 50 wt %, or 5 wt % to 45 wt %, on the basis of the total weight of the pesticide composition. Specifically, the concentration of the one or more compounds selected from the group consisting of the compound represented by Formula 1 above, a stereoisomer thereof, a hydrate thereof, and a salt thereof, as an active ingredient(s), may be 0.01 ppm to 1,000 ppm, 0.03 ppm to 500 ppm, 0.05 ppm to 300 ppm, 0.1 ppm to 200 ppm, 0.1 ppm to 100 ppm, 0.1 ppm to 50 ppm, 0.1 ppm to 10 ppm, 0.1 ppm to 5 ppm, or 1 ppm to 5 ppm, but is not limited thereto.

Additionally, the compound represented by Formula 1 above included in the pesticide composition according to an embodiment of the present invention may be used at a rate of 0.1 g to 10 kg, 1 g to 6 kg, or 1 g to 1 kg per 1 hectare (Ha) for pest control.

The pesticide composition according to an embodiment of the present invention not only exhibits excellent pesticidal activity against the above-described pests, but also exhibits pesticidal activity within a short period of time (e.g., 24 hours), thereby enabling a more efficient pest control.

Additionally, the pesticide composition according to an embodiment of the present invention, when applied to plants, may effectively reduce the feeding area on the plants, thereby being capable of preventing pests from feeding on the plants while controlling pests.

### Method for Controlling Pests

The present invention provides a method for controlling pests using the above-described pesticide composition. Specifically, the method includes treating crops or their habitats with the pesticide composition.

According to an embodiment of the present invention, the treating of the pesticide composition to crops or their habitats may specifically include spraying the pesticide composition, allowing the pesticide composition to be in contact, dipping the pesticide composition, etc.

Through the pest control method according to the present invention, harmful pests can be controlled efficiently.

### MODE FOR THE INVENTION

Hereinafter, the present invention is described in more detail with the following examples. However, the scope of the present invention is not limited to these examples.

The abbreviations used in Preparation Examples and Synthesis Examples below are summarized as follows.
THF: Tetrahydrofuran
TLC: Thin Layer Chromatography
NCS: N-Chlorosuccinimide
EA: Ethyl Acetate
DMF: Dimethylformamide
MC: Methylene chloride
DMAP: Dimethylaminopyridine
DIPEA: Diisopropylethylamine
EDC: 1-Ethyl-3-(3-dimethylaminopropyl)carbodiimide

### Preparation Example 1

### 1) Synthesis of methyl (Z)-4-((hydroxvimino)methyl)-2-methylbenzoate

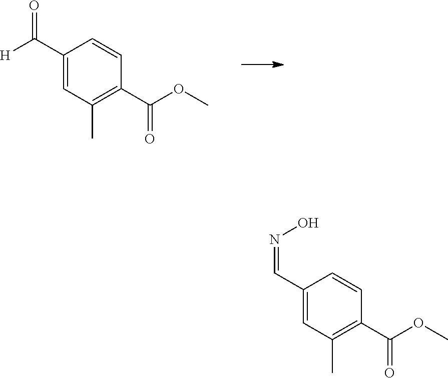

Hydroxylamine hydrochloride (NH₂OH HCl, 101.60 g, 1347.00 mmol) and sodium acetate (NaOAc, 120.00 g, 1347.00 mmol) were dissolved in THF (1,104 mL)/H₂O (1,104 mL). Then, methyl 4-formyl-2-methylbenzoate (120.00 g, 673.00 mmol) and THF (1,104 mL) were slowly added thereto at 0° C. and the mixture was stirred at room temperature for about 1 hour, and the completion of the reaction was confirmed by TLC, and the resultant was extracted with H₂O/EA. Then, the organic layer was concentrated and then purified with EA/hexane to obtain methyl (Z)-4-((hydroxyimino)methyl)-2-methylbenzoate (110 g).

¹H NMR (500 MHz, DMSO-d₆): δ 11.52 (s, 1H), 8.15 (s, 1H), 7.84 (d, J=8.4 Hz, 1H), 7.54-7.51 (m, 2H), 3.82 (s, 3H), 2.52 (s, 3H).

### 2) Synthesis of methyl 4-(5-(3-chloro-5-(trifluoromethyl)phenyl)-5-(trifluoromethyl)-4,5-dihydroisoxazol-3-yl)-2-methylbenzoate

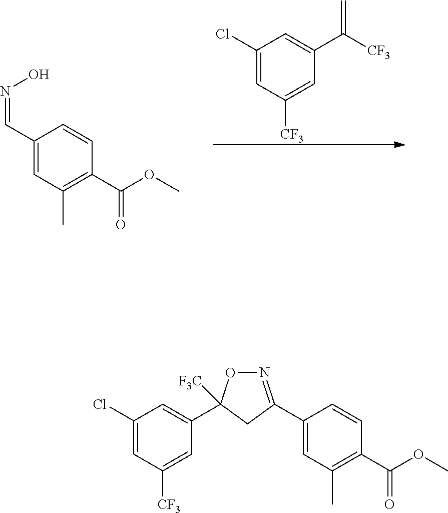 [SMILES: COC(=O)c1ccc(/C=N\O)cc1C ; C=C(c1cc(Cl)cc(C(F)(F)F)c1)C(F)(F)F ; COC(=O)c1ccc(C2=NOC(c3cc(Cl)cc(C(F)(F)F)c3)(C(F)(F)F)C2)cc1C]

The methyl (Z)-4-((hydroxyimino)methyl)-2-methylbenzoate (220.00 g, 1,138.00 mmol) synthesized in step 1) above was dissolved in DMF (7,966 mL). Then, NCS (167.00 g, 1,252.00 mmol) was added thereto, and the temperature was raised to 55° C., and the mixture was stirred for about 1 hour. Then, 1-chloro-3-(trifluoromethyl)-5-(3,3,3-trifluoroprop-1-en-2-yl)benzene (328.10 g, 1,195.00 mmol) was added thereto and the mixture was stirred at room temperature for about 12 hours. Thereafter, the completion of the reaction was confirmed by TLC, and the resultant was extracted with H₂O/EA. Then, the organic layer was dried over anhydrous sodium sulfate (Na₂SO₄) and the pressure was reduced to remove the solvent, to thereby obtain methyl 4-(5-(3-chloro-5-(trifluoromethyl)phenyl)-5-(trifluoromethyl)-4,5-dihydroisoxazol-3-yl)-2-methylbenzoate (429 g).

¹H NMR (500 MHz, DMSO-d₆): δ 8.09 (s, 1H), 7.97 (s, 1H), 7.90 (d, J=7.9 Hz, 1H), 7.85 (s, 1H), 7.71-7.66 (m, 2H), 4.51-4.34 (m, 2H), 3.85 (s, 3H), 2.55 (s, 3H).

### 3) Synthesis of 4-(5-(3-chloro-5-(trifluoromethyl)phenvl)-5-(trifluoromethyl)-4,5-dihydroisoxazol-3-yl)-2-methylbenzoic acid

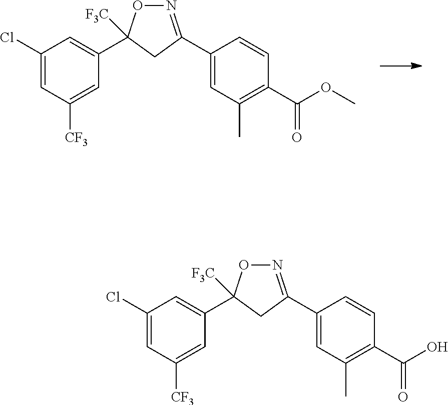 [SMILES: COC(=O)c1ccc(C2=NOC(c3cc(Cl)cc(C(F)(F)F)c3)(C(F)(F)F)C2)cc1C ; Cc1cc(C2=NOC(c3cc(Cl)cc(C(F)(F)F)c3)(C(F)(F)F)C2)ccc1C(=O)O]

The methyl 4-(5-(3-chloro-5-(trifluoromethyl)phenyl)-5-(trifluoromethyl)-4,5-dihydroisoxazol-3-yl)-2-methylbenzoate(429.00 g, 922.00 mmol) synthesized in step 2) above and THF (4,611 mL) were mixed, and then potassium hydroxide (KOH, 134.50 g, 2,398.00 mmol)/H₂O (4,611 mL) was slowly added thereto. After stirring the mixture in a reflux state for about 1 hour, the completion of the reaction was confirmed by TLC, and the resultant was extracted with H₂O/EA. Then, the organic layer was dried over anhydrous sodium sulfate (Na₂SO₄) and the pressure was reduced to remove the solvent. Thereafter, the resultant was recrystallized with hexane, and then dehydrated to obtain 4-(5-(3-chloro-5-(trifluoromethyl)phenyl)-5-(trifluoromethyl)-4,5-dihydroisoxazol-3-yl)-2-methylbenzoic acid (229.1 g).

¹H NMR (500 MHz, DMSO-d₆): δ 13.13 (s, 1H), 8.09 (t, J=2.1 Hz, 1H), 7.98 (t, J=1.9 Hz, 1H), 7.91-7.87 (m, 1H), 7.85 (s, 1H), 7.65 (dd, J=8.5, 1.4 Hz, 2H), 4.49-4.35 (m, 2H), 2.55 (s, 3H).

### 4) Synthesis of 4-(5-(3-chloro-5-(trifluoromethyl)phenyl)-5-(trifluoromethyl)-4,5-dihydroisoxazol-3-yl)-2-methylbenzamide(SM2)

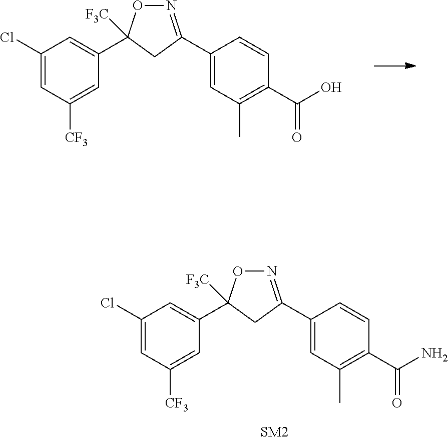 [SMILES: Cc1cc(C2=NOC(c3cc(Cl)cc(C(F)(F)F)c3)(C(F)(F)F)C2)ccc1C(=O)O ; Cc1cc(C2=NOC(c3cc(Cl)cc(C(F)(F)F)c3)(C(F)(F)F)C2)ccc1C(N)=O]

The 4-(5-(3-chloro-5-(trifluoromethyl)phenyl)-5-(trifluoromethyl)-4,5-dihydroisoxazol-3-yl)-2-methylbenzoic acid (25.00 g, 55.34 mmol) synthesized in step 3) above and SOC₃ (65.80 g, 553.40 mmol) were mixed, and the mixture reacted in a reflux state for about 2 hours and was concentrated. Then, the resultant was diluted in MC (50 mL), and ammonia water (NH₂OH, 100 mL) was slowly added thereto at 0° C. After confirming the formation of solids, the resultant was purified with H₂O/hexane to obtain 4-(5-(3-chloro-5-(trifluoromethyl)phenyl)-5-(trifluoromethyl)-4,5-dihydroisoxazol-3-yl)-2-methylbenzamide (23 g).

¹H NMR (500 MHz, DMSO-d₆): δ 8.09 (s, 1H), 7.98 (s, 1H), 7.86 (s, 1H), 7.82 (s, 1H), 7.62-7.56 (m, 2H), 7.49 (s, 1H), 7.46 (d, J=7.9 Hz, 1H), 4.48-4.32 (m, 2H), 2.40 (s, 3H).

### Preparation Examples 2 to 11

Steps 1) to 4) of Preparation Example 1 were performed in the same manner, but the reactants in each step were changed to respectively synthesize Compounds SM1, SM3 to SM11 below.

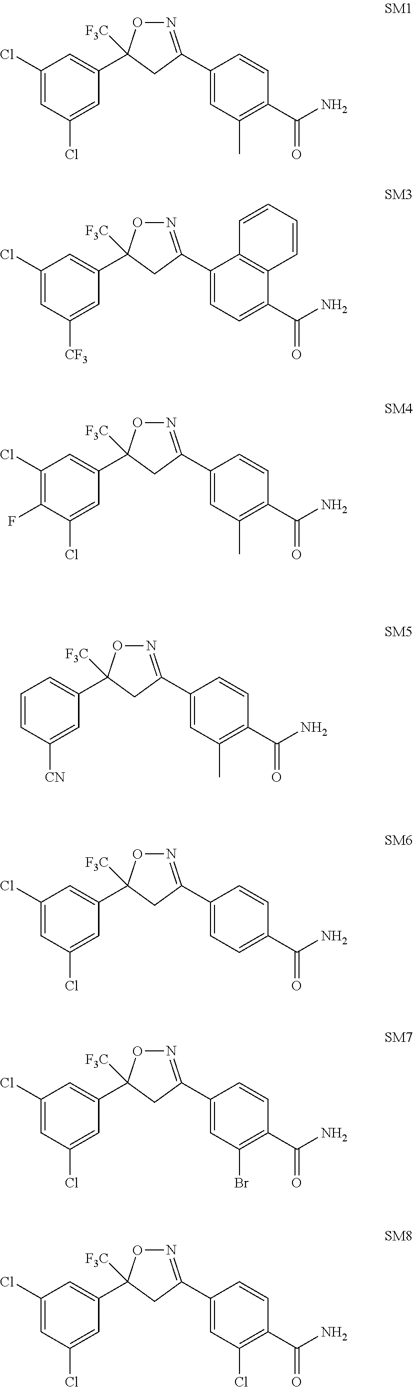

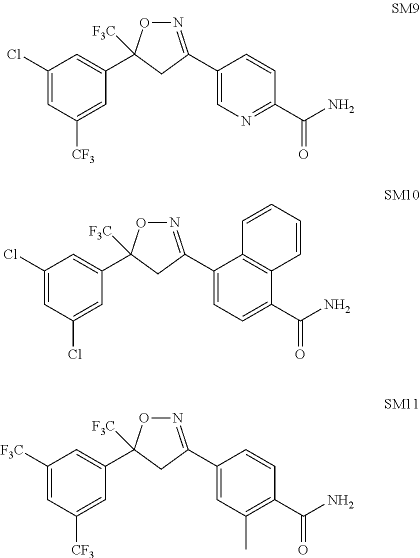 [SMILES: NC(=O)c1ccc(C2=NOC(c3cc(Cl)cc(C(F)(F)F)c3)(C(F)(F)F)C2)cn1 ; NC(=O)c1ccc(C2=NOC(c3cc(Cl)cc(Cl)c3)(C(F)(F)F)C2)c2ccccc12 ; Cc1cc(C2=NOC(c3cc(C(F)(F)F)cc(C(F)(F)F)c3)(C(F)(F)F)C2)ccc1C(N)=O]

### Synthesis Example 1—Synthesis of Compound 1001

### 1) Synthesis of Compound 1001 (R/S)

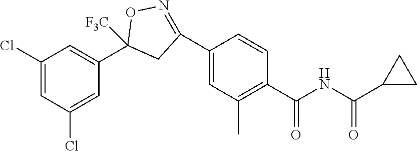 [SMILES: Cc1cc(C2=NOC(c3cc(Cl)cc(Cl)c3)(C(F)(F)F)C2)ccc1C(=O)NC(=O)C1CC1]

The SM1 (0.50 g, 1.19 mmol) synthesized in Preparation Examples above was dissolved in THF (10 mL) and then added to sodium hydride (NaH, 0.09 g, 2.35 mmol) at 0° C. Then, the mixture was stirred at room temperature for 2 hours, and then cyclopropanecarbonyl chloride (0.13 g, 1.08 mmol) was added thereto, and they were reacted at room temperature for 4 hours. After confirming the completion of the reaction by TLC, ammonium chloride (NH₄Cl) was added thereto at 0° C. and then the mixture was extracted with EA. Then, the organic layer was dried over anhydrous Na₂SO₄ and then removed the solvent by reducing the pressure. Thereafter, the reaction product was purified by silica gel column chromatography to obtain Compound 1001 (0.3 g).

¹H NMR (500 MHz, DMSO-d₆): δ 11.34 (s, 1H), 7.82 (t, J=1.9 Hz, 1H), 7.68-7.58 (m, 4H), 7.49 (d, J=8.0 Hz, 1H), 4.37 (s, 1H), 4.32 (s, 1H), 2.36 (s, 3H), 2.20 (tt, J=7.8, 4.5 Hz, 1H), 0.94-0.86 (m, 2H), 0.85 (dt, J=4.4, 2.9 Hz, 2H).

### 2) Synthesis of Compound 1001 (S)

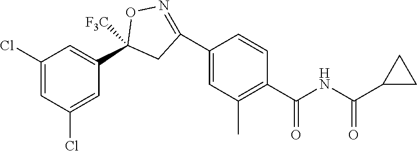 [SMILES: Cc1cc(C2=NO[C@@](c3cc(Cl)cc(Cl)c3)(C(F)(F)F)C2)ccc1C(=O)NC(=O)C1CC1]

Compound 1001 (S) was obtained through the same process as step 1) above using (S)-4-(5-(3,5-dichlorophenyl)-5-(trifluoromethyl)-4,5-dihydroisoxazol-3-yl)-2-methylbenzoic acid.

¹H NMR (500 MHz, DMSO-d₆): δ 11.34 (s, 1H), 7.82 (t, J=1.9 Hz, 1H), 7.68-7.58 (m, 4H), 7.49 (d, J=8.0 Hz, 1H), 4.37 (s, 1H), 4.32 (s, 1H), 2.36 (s, 3H), 2.20 (tt, J=7.8, 4.5 Hz, 1H), 0.94-0.86 (m, 2H), 0.85 (dt, J=4.4, 2.9 Hz, 2H).

### 3) Synthesis of Compound 1001 (R)

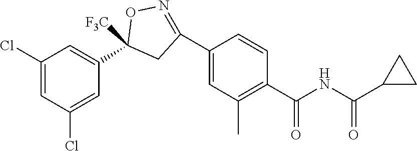 [SMILES: Cc1cc(C2=NO[C@](c3cc(Cl)cc(Cl)c3)(C(F)(F)F)C2)ccc1C(=O)NC(=O)C1CC1]

Compound 1001 (R) was obtained through the same process as step 1) above using (R)-4-(5-(3,5-dichlorophenyl)-5-(trifluoromethyl)-4,5-dihydroisoxazol-3-yl)-2-methylbenzoic acid.

¹H NMR (500 MHz, DMSO-d₆): δ 11.34 (s, 1H), 7.82 (t, J=1.9 Hz, 1H), 7.68-7.58 (m, 4H), 7.49 (d, J=8.0 Hz, 1H), 4.37 (s, 1H), 4.32 (s, 1H), 2.36 (s, 3H), 2.20 (tt, J=7.8, 4.5 Hz, 1H), 0.94-0.86 (m, 2H), 0.85 (dt, J=4.4, 2.9 Hz, 2H).

### Synthesis Example 2—Synthesis of Compound 1002

### 1) Synthesis of Compound 1002 (R/S)

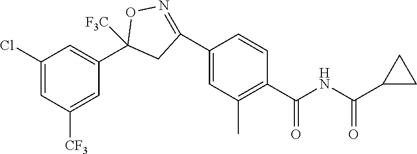 [SMILES: Cc1cc(C2=NOC(c3cc(Cl)cc(C(F)(F)F)c3)(C(F)(F)F)C2)ccc1C(=O)NC(=O)C1CC1]

The SM2 (10.00 g, 22.13 mmol) synthesized in Preparation Examples above was dissolved in THF (200 mL) and then added to NaH (1.77 g, 44.26 mmol) and mixed. Then, the mixture was stirred at room temperature for 2 hours, and cyclopropanecarbonyl chloride (2.10 g, 20.12 mmol) was added thereto, and were reacted at room temperature for 4 hours. After confirming the completion of the reaction by TLC, NH₄Cl was added thereto at 0° C. and then the mixture was extracted with EA. Then, the organic layer was dried over anhydrous Na₂SO₄ and then removed the solvent by reducing the pressure. Thereafter, the reaction product was purified by silica gel column chromatography to obtain Compound 1002 (6 g).

¹H NMR (500 MHz, DMSO-d₆): δ 11.35 (s, 1H), 8.09 (td, J=1.7, 0.8 Hz, 1H), 7.98 (d, J=1.9 Hz, 1H), 7.86 (s, 1H), 7.66-7.59 (m, 2H), 7.50 (d, J=7.9 Hz, 1H), 4.43 (s, 1H), 4.39 (s, 1H), 2.36 (s, 3H), 2.20 (tt, J=7.2, 4.6 Hz, 1H), 0.94-0.86 (m, 2H), 0.88-0.80 (m, 2H).

### 2) Synthesis of Compound 1002 (S)

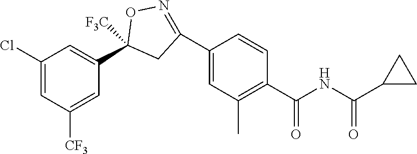 [SMILES: Cc1cc(C2=NO[C@@](c3cc(Cl)cc(C(F)(F)F)c3)(C(F)(F)F)C2)ccc1C(=O)NC(=O)C1CC1]

Compound 1002 (S) was obtained through the same process as step 1) above using (S)-4-(5-(3-chloro-5-(trifluoromethyl)phenyl)-5-(trifluoromethyl)-4,5-dihydroisoxazol-3-yl)-2-methylbenzoic acid.

¹H NMR (500 MHz, DMSO-d₆): δ 11.35 (s, 1H), 8.09 (td, J=1.7, 0.8 Hz, 1H), 7.98 (d, J=1.9 Hz, 1H), 7.86 (s, 1H), 7.66-7.59 (m, 2H), 7.50 (d, J=7.9 Hz, 1H), 4.43 (s, 1H), 4.39 (s, 1H), 2.36 (s, 3H), 2.20 (tt, J=7.2, 4.6 Hz, 1H), 0.94-0.86 (m, 2H), 0.88-0.80 (m, 2H).

### 3) Synthesis of Compound 1002 (R)

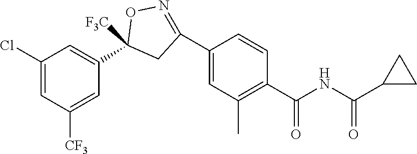 [SMILES: Cc1cc(C2=NO[C@](c3cc(Cl)cc(C(F)(F)F)c3)(C(F)(F)F)C2)ccc1C(=O)NC(=O)C1CC1]

Compound 1002 (R) was obtained through the same process as step 1) above using (R)-4-(5-(3-chloro-5-(trifluoromethyl)phenyl)-5-(trifluoromethyl)-4,5-dihydroisoxazol-3-yl)-2-methylbenzoic acid.

¹H NMR (500 MHz, DMSO-d₆): δ 11.35 (s, 1H), 8.09 (td, J=1.7, 0.8 Hz, 1H), 7.98 (d, J=1.9 Hz, 1H), 7.86 (s, 1H), 7.66-7.59 (m, 2H), 7.50 (d, J=7.9 Hz, 1H), 4.43 (s, 1H), 4.39 (s, 1H), 2.36 (s, 3H), 2.20 (tt, J=7.2, 4.6 Hz, 1H), 0.94-0.86 (m, 2H), 0.88-0.80 (m, 2H).

### Synthesis Example 3—Synthesis of Compound 1003

### 1) Synthesis of Compound 1003 (R/S)

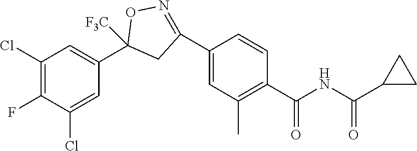 [SMILES: Cc1cc(C2=NOC(c3cc(Cl)c(F)c(Cl)c3)(C(F)(F)F)C2)ccc1C(=O)NC(=O)C1CC1]

Compound 1003 (R/S) was obtained through the same process as in Synthesis Example 1, except that SM4 was used in place of SM1.

¹H NMR (500 MHz, DMSO-d₆): δ 11.35 (s, 1H), 7.81 (d, J=6.1 Hz, 2H), 7.65-7.58 (m, 2H), 7.50 (d, J=8.0 Hz, 1H), 4.40-4.28 (m, 2H), 2.36 (s, 3H), 2.20 (tt, J=7.6, 4.5 Hz, 1H), 0.91 (ddt, J=7.5, 5.6, 2.3 Hz, 2H), 0.85 (dt, J=4.5, 2.9 Hz, 2H).

### 2) Synthesis of Compound 1003 (S)

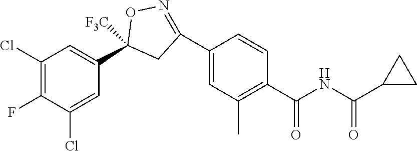 [SMILES: Cc1cc(C2=NO[C@@](c3cc(Cl)c(F)c(Cl)c3)(C(F)(F)F)C2)ccc1C(=O)NC(=O)C1CC1]

Compound 1003 (S) was obtained through the same process as step 1) above using (S)-4-(5-(3,5-dichloro-4-fluorophenyl)-5-(trifluoromethyl)-4,5-dihydroisoxazol-3-yl)-2-methylbenzoic acid.

¹H NMR (500 MHz, DMSO-d₆): δ 11.35 (s, 1H), 7.81 (d, J=6.1 Hz, 2H), 7.65-7.58 (m, 2H), 7.50 (d, J=8.0 Hz, 1H), 4.40-4.28 (m, 2H), 2.36 (s, 3H), 2.20 (tt, J=7.6, 4.5 Hz, 1H), 0.91 (ddt, J=7.5, 5.6, 2.3 Hz, 2H), 0.85 (dt, J=4.5, 2.9 Hz, 2H).

### 3) Synthesis of Compound 1003 (R)

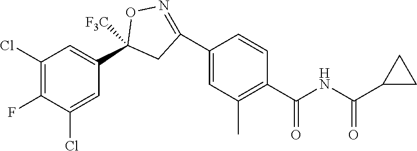 [SMILES: Cc1cc(C2=NO[C@@](c3cc(Cl)c(F)c(Cl)c3)(C(F)(F)F)C2)ccc1C(=O)NC(=O)C1CC1]

Compound 1003 (R) was obtained through the same process as step 1) above using (R)-4-(5-(3,5-dichloro-4-fluorophenyl)-5-(trifluoromethyl)-4,5-dihydroisoxazol-3-yl)-2-methylbenzoic acid.

¹H NMR (500 MHz, DMSO-d₆): δ 11.35 (s, 1H), 7.81 (d, J=6.1 Hz, 2H), 7.65-7.58 (m, 2H), 7.50 (d, J=8.0 Hz, 1H), 4.40-4.28 (m, 2H), 2.36 (s, 3H), 2.20 (tt, J=7.6, 4.5 Hz, 1H), 0.91 (ddt, J=7.5, 5.6, 2.3 Hz, 2H), 0.85 (dt, J=4.5, 2.9 Hz, 2H).

### Synthesis Example 4—Synthesis of Compound 1004

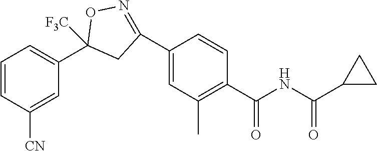 [SMILES: Cc1cc(C2=NOC(c3cccc(C#N)c3)(C(F)(F)F)C2)ccc1C(=O)NC(=O)C1CC1]

Compound 1004 was obtained through the same process as in Synthesis Example 1, except that SM5 was used instead of SM1.

¹H NMR (500 MHz, DMSO-d₆): δ 11.36 (s, 1H), 8.08-8.03 (m, 1H), 8.00 (dt, J=7.8, 1.4 Hz, 1H), 7.96 (d, J=8.4 Hz, 1H), 7.76 (t, J=7.9 Hz, 1H), 7.66-7.60 (m, 2H), 7.49 (d, J=8.1 Hz, 1H), 4.42 (d, J=18.3 Hz, 1H), 4.31 (d, J=18.3 Hz, 1H), 2.35 (s, 3H), 2.20 (ddd, J=12.4, 7.8, 4.7 Hz, 1H), 0.90 (dq, J=7.8, 2.5 Hz, 2H), 0.85 (dq,J=5.8, 2.7 Hz, 2H).

### Synthesis Example 5—Synthesis of Compound 1005

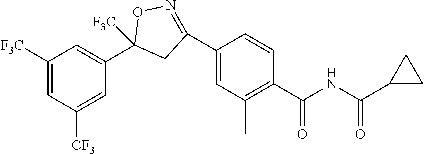 [SMILES: Cc1cc(C2=NOC(c3cc(C(F)(F)F)cc(C(F)(F)F)c3)(C(F)(F)F)C2)ccc1C(=O)NC(=O)C1CC1]

Compound 1005 was obtained through the same process as in Synthesis Example 1, except that SM11 was used in place of SM1.

¹H NMR (500 MHz, DMSO-d₆): δ 11.36 (s, 1H), 8.35 (s, 1H), 8.21 (d, J=1.2 Hz, 2H), 7.64 (d, J=2.1 Hz, 1H), 7.64-7.60 (m, 1H), 7.50 (d, J=7.9 Hz, 1H), 4.48 (q, J=18.4 Hz, 2H), 2.36 (s, 3H), 2.20 (tt, J=7.8, 4.6 Hz, 1H), 0.90 (dt, J=7.6, 3.0 Hz, 2H), 0.85 (dt, J=4.4, 2.9 Hz, 2H).

### Synthesis Example 6—Synthesis of Compound 1006

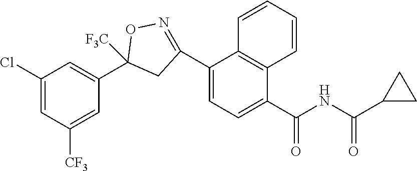 [SMILES: O=C(NC(=O)C1CC1)c1ccc(C2=NOC(c3cc(Cl)cc(C(F)(F)F)c3)(C(F)(F)F)C2)c2ccccc12]

Compound 1006 was obtained through the same process as in Synthesis Example 1, except that SM3 was used in place of of SM1.

¹H NMR (500 MHz, DMSO-d₆): δ 11.63 (s, 1H), 8.84-8.76 (m, 1H), 8.11 (d, J=1.8 Hz, 1H), 8.09-8.03 (m, 2H), 7.95-7.85 (m, 2H), 7.83-7.64 (m, 3H), 4.62 (s, 2H), 2.29-2.18 (m, 1H), 0.93 (ddt, J=7.7, 6.0, 2.8 Hz, 2H), 0.87-0.83 (m, 2H).

### Synthesis Example 7—Synthesis of Compound 1007

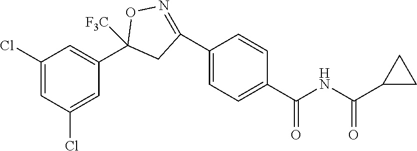 [SMILES: O=C(NC(=O)C1CC1)c1ccc(C2=NOC(c3cc(Cl)cc(Cl)c3)(C(F)(F)F)C2)cc1]

Compound 1007 was obtained through the same process as in Synthesis Example 1, except that SM6 was used in place of SM1.

¹H NMR (500 MHz, DMSO-d₆): δ 11.26 (s, 1H), 8.01 (d, J=1.9 Hz, 1H), 7.99 (d, J=1.7 Hz, 1H), 7.87 (d, J=3.8 Hz, 1H), 7.85 (d, J=1.7 Hz, 1H), 7.82 (q, J=1.9 Hz, 1H), 7.64 (d, J=1.9 Hz, 2H), 4.44 (d, J=18.3 Hz, 1H), 4.38-4.30 (m, 1H), 2.48-2.45 (m, 1H), 0.93-0.92 (m, 2H), 0.87-0.84 (m, 2H).

### Synthesis Example 8—Synthesis of Compound 1008

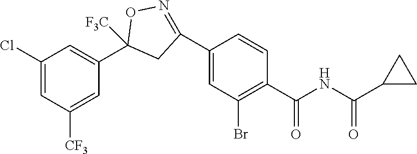 [SMILES: O=C(NC(=O)C1CC1)c1ccc(C2=NOC(c3cc(Cl)cc(C(F)(F)F)c3)(C(F)(F)F)C2)cc1Br]

Compound 1008 was obtained through the same process as in Synthesis Example 1, except that SM7 was used in place of SM1.

¹H NMR (500 MHz, CDCl₃): δ 8.42 (s, 1H), 7.97-7.91 (m, 1H), 7.79-7.71 (m, 1H), 7.57 (d, J=8.0 Hz, 1H), 7.52 (d, J=1.8 Hz, 2H), 7.47 (t, J=1.9 Hz, 1H), 4.13-4.05 (m, 1H), 3.71 (dd, J=17.3, 3.8 Hz, 1H), 2.51 (s, 1H), 1.21 (dt, J=4.5, 3.3 Hz, 2H), 1.10-1.07 (m, 2H).

### Synthesis Example 9—Synthesis of Compound 1009

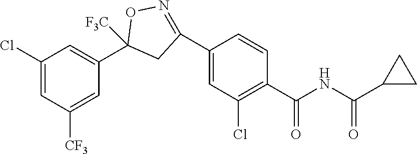 [SMILES: O=C(NC(=O)C1CC1)c1ccc(C2=NOC(c3cc(Cl)cc(C(F)(F)F)c3)(C(F)(F)F)C2)cc1Cl]

Compound 1009 was obtained through the same process as in Synthesis Example 1, except that SM8 was used in place of SM1.

¹H NMR (500 MHz, CDCl₃): δ 8.52 (s, 1H), 7.66 (d, J=1.5 Hz, 1H), 7.65-7.51 (m, 2H), 7.43 (d, J=1.8 Hz, 2H), 7.37 (t, J=1.8 Hz, 1H), 3.99 (d, J=17.2 Hz, 1H), 3.62 (d, J=17.2 Hz, 1H), 2.46 (s, 1H), 1.12 (dd, J=4.4, 3.2 Hz, 2H), 1.00-0.97 (m, 2H).

### Synthesis Example 10—Synthesis of Compound 1010

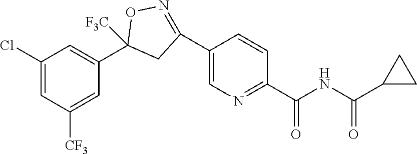 [SMILES: O=C(NC(=O)C1CC1)c1ccc(C2=NOC(c3cc(Cl)cc(C(F)(F)F)c3)(C(F)(F)F)C2)cn1]

Compound 1010 was obtained through the same process as in Synthesis Example 1, except that SM9 was used in place of SM1.

¹H NMR (500 MHz, CDCl₃): δ 10.37 (s, 1H), 8.86 (dd, J=2.1, 0.9 Hz, 1H), 8.35 (dd, J=8.2, 0.8 Hz, 1H), 8.22 (dd, J=8.2, 2.1 Hz, 1H), 7.80 (s, 1H), 7.74 (s, 1H), 7.70 (s, 1H), 4.17 (d, J=17.2 Hz, 1H), 3.76 (d, J=17.2 Hz, 1H), 2.99 (tt, J=7.9, 4.6 Hz, 1H), 1.22 (dd, J=4.6, 3.2 Hz, 2H), 1.05 (dt, J=8.1, 3.3 Hz, 2H).

### Synthesis Example 11—Synthesis of Compound 1011

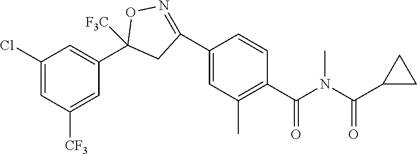 [SMILES: Cc1cc(C2=NOC(c3cc(Cl)cc(C(F)(F)F)c3)(C(F)(F)F)C2)ccc1C(=O)N(C)C(=O)C1CC1]

4-(5-(3-chloro-5-(trifluoromethyl)phenyl)-5-(trifluoromethyl)-4,5-dihydroisoxazol-3-yl)-2-methylbenzoic acid (0.50 g, 1.20 mmol) was diluted in MC (10 mL), and EDC (0.46 g, 2.40 mmol) and DMAP (0.03 g, 0.24 mmol) were added thereto, and methyl amine (0.06 g, 1.80 mmol) was injected thereto. Then, the mixture was stirred at room temperature for 12 hours, and after confirming the completion of the reaction by TLC, the resultant was extracted with MC/sodium bicarbonate (NaHCO₃). Then, the organic layer was dried over anhydrous Na₂SO₄ and the solvent was removed by reducing the pressure. Thereafter, the reaction product was purified by silica gel column chromatography to obtain a compound (0.4 g).

The obtained compound (0.05 g) was diluted in MC (0.11 mL), and then DIPEA (0.03 mL, 0.16 mmol) and cyclopropanecarbonyl chloride (0.02 mL, 0.16 mmol) were added thereto at 0° C., and then reacted at room temperature for 12 hours. After confirming the completion of the reaction by TLC, the resultant was extracted with MC/NaHCO₃. Then, the organic layer was dried over anhydrous Na₂SO₄ and the solvent was removed by reducing the pressure. Thereafter, the reaction product was purified by silica gel column chromatography to obtain Compound 1011 (0.03 g).

¹H NMR (500 MHz, CDCl₃): δ 7.80 (s, 1H), 7.74 (s, 1H), 7.67 (s, 1H), 7.54 (dt, J=1.5, 0.7 Hz, 1H), 7.54-7.50 (m, 1H), 7.31 (d, J=7.9 Hz, 1H), 4.12 (d, J=17.2 Hz, 1H), 3.70 (d, J=17.2 Hz, 1H), 3.27 (s, 3H), 2.39 (s, 3H), 1.91 (tt, J=7.8, 4.6 Hz, 1H), 1.04 (dq, J=4.6, 3.7 Hz, 2H), 0.80-0.76 (m, 2H).

### Synthesis Example 12—Synthesis of Compound 1012

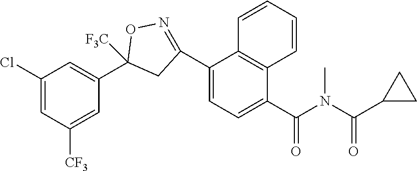 [SMILES: CN(C(=O)c1ccc(C2=NOC(c3cc(Cl)cc(C(F)(F)F)c3)(C(F)(F)F)C2)c2ccccc12)C(=O)C1CC1]

Compound 1012 was obtained through the same process as in Synthesis Example 11, except that 4-(5-(3-chloro-5-(trifluoromethyl)phenyl)-5-(trifluoromethyl)-4,5-dihydroisoxazol-3-yl)-1-naphthoic acid was used 4-(5-(3-chloro-5-(trifluoromethyl)phenyl)-5-(trifluoromethyl)-4,5-dihydroisoxazol-3-yl)-2-methylbenzoic acid in place of 4-(5-(3-chloro-5-(trifluoromethyl)phenyl)-5-(trifluoromethyl)-4,5-dihydroisoxazol-3-yl)-2-methylbenzoic acid.

¹H NMR (500 MHz, CDCl₃): δ 8.91-8.88 (m, 1H), 8.05-8.01 (m, 1H), 7.85 (s, 1H), 7.79 (s, 1H), 7.72-7.69 (m, 1H), 7.69-7.62 (m, 2H), 7.54 (d, J=7.5 Hz, 1H), 7.50 (d, J=7.5 Hz, 1H), 4.32 (d, J=17.2 Hz, 1H), 3.92 (d, J=17.2 Hz, 1H), 3.31 (s, 3H), 1.95 (tt, J=7.9, 4.7 Hz, 1H), 0.99-0.96 (m, 2H), 0.67 (dd, J=7.5, 3.7 Hz, 2H).

### Synthesis Example 13—Synthesis of Compound 1013

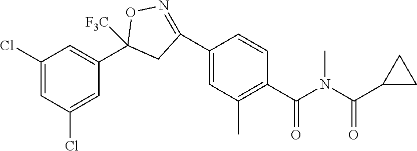 [SMILES: Cc1cc(C2=NOC(c3cc(Cl)cc(Cl)c3)(C(F)(F)F)C2)ccc1C(=O)N(C)C(=O)C1CC1]

Compound 1013 was obtained through the same process as in Synthesis Example 11, except that 4-(5-(3,5-dichlorophenyl)-5-(trifluoromethyl)-4,5-dihydroisoxazol-3-yl)-2-methylbenzoic acid was used in place of 4-(5-(3-chloro-5-(trifluoromethyl)phenyl)-5-(trifluoromethyl)-4,5-dihydroisoxazol-3-yl)-2-methylbenzoic acid.

¹H NMR (500 MHz, CDCl₃): δ 7.55-7.47 (m, 4H), 7.41 (t, J=1.8 Hz, 1H), 7.31 (d, J=7.9 Hz, 1H), 4.06 (d, J=17.1 Hz, 1H), 3.67 (d, J=17.2 Hz, 1H), 3.27 (s, 3H), 2.39 (s, 3H), 1.90 (tt, J=7.8, 4.6 Hz, 1H), 1.06-1.02 (m, 2H), 0.80-0.74 (m, 2H).

### Synthesis Example 14—Synthesis of Compound 1014

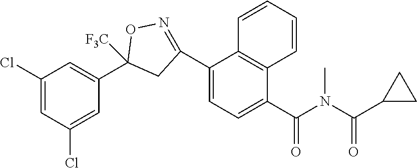 [SMILES: CN(C(=O)c1ccc(C2=NOC(c3cc(Cl)cc(Cl)c3)(C(F)(F)F)C2)c2ccccc12)C(=O)C1CC1]

Compound 1014 was obtained through the same process as in Synthesis Example 11, except that SM10 was used in place of 4-(5-(3-chloro-5-(trifluoromethyl)phenyl)-5-(trifluoromethyl)-4,5-dihydroisoxazol-3-yl)-2-methylbenzoic acid.

¹H NMR (500 MHz, CDCl₃): δ 8.89 (dt, J=8.9, 1.0 Hz, 1H), 8.05-8.01 (m, 1H), 7.66 (dddd, J=19.5, 8.2, 6.9, 1.4 Hz, 2H), 7.56-7.47 (m, 4H), 7.44 (t, J=1.9 Hz, 1H), 4.26 (d, J=17.2 Hz, 1H), 3.89 (d, J=17.2 Hz, 1H), 3.31 (s, 3H), 1.94 (tt, J=7.8, 4.6 Hz, 1H), 0.97 (dd, J=4.6, 3.4 Hz, 2H), 0.68-0.63 (m, 2H).

### Synthesis Example 15—Synthesis of Compound 1015

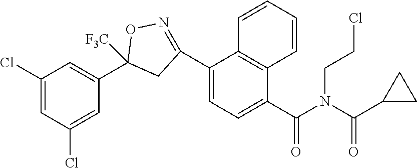 [SMILES: O=C(c1ccc(C2=NOC(c3cc(Cl)cc(Cl)c3)(C(F)(F)F)C2)c2ccccc12)N(CCCl)C(=O)C1CC1]

4-(5-(3,5-dichlorophenyl)-5-(trifluoromethyl)-4,5-dihydroisoxazol-3-yl)-1-naphthoic acid (0.25 g, 0.50 mmol) was diluted in MC (5 mL), and DIPEA (0.13 mL, 0.75 mmol) was added thereto at 0° C., and then cyclopropanecarbonyl chloride (0.07 mL, 0.75 mmol) was injected thereto. Then, the mixture was stirred at room temperature for 12 hours, and after confirming the completion of the reaction by TLC, the resultant was extracted with MC/H₂O. Then, the organic layer was dried over anhydrous Na₂SO₄ and the solvent was removed by reducing the pressure. Thereafter, the reaction product was purified by silica gel column chromatography to obtain Compound 1015 (0.012 g).

¹H NMR (500 MHz, CDCl₃): δ 8.88 (dd, J=7.7, 2.4 Hz, 1H), 8.21-8.15 (m, 1H), 7.72-7.64 (m, 2H), 7.63-7.59 (m, 1H), 7.56-7.50 (m, 3H), 7.44 (q, J=2.0 Hz, 1H), 4.27 (d, J=6.0 Hz, 2H), 4.24 (d, J=5.2 Hz, 1H), 3.89 (d, J=17.4 Hz, 1H), 3.84 (t, J=6.1 Hz, 2H), 1.28 (d, J=6.7 Hz, 3H), 1.10 (d, J=6.9 Hz, 2H).

### Synthesis Example 16—Synthesis of Compound 1016

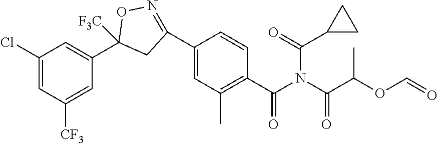 [SMILES: Cc1cc(C2=NOC(c3cc(Cl)cc(C(F)(F)F)c3)(C(F)(F)F)C2)ccc1C(=O)N(C(=O)C1CC1)C(=O)C(C)OC=O]

Compound 1016 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.99 (s, 1H), 7.57-7.46 (m, 4H), 7.41 (t, J=1.8 Hz, 1H), 7.31 (d, J=7.9 Hz, 1H), 5.44-5.36 (m, 1H), 4.13-4.03 (m, 2H), 3.95 (dd, J=14.3, 3.1 Hz, 1H), 3.67 (d, J=17.2 Hz, 1H), 3.37 (q, J=7.2 Hz, 1H), 2.42 (s, is 3H), 1.70-1.62 (m, 2H), 1.62-1.47 (m, 2H), 1.30 (d, J=6.4 Hz, 3H).

### Synthesis Example 17—Synthesis of Compound 1017

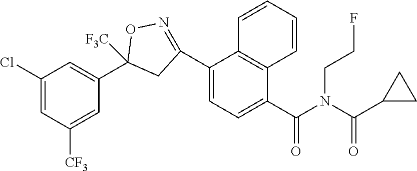 [SMILES: O=C(c1ccc(C2=NOC(c3cc(Cl)cc(C(F)(F)F)c3)(C(F)(F)F)C2)c2ccccc12)N(CCF)C(=O)C1CC1]

Compound 1017 was obtained through the same process as in Synthesis Example 15 except that 4-(5-(3-chloro-5-(trifluoromethyl)phenyl)-5-(trifluoromethyl)-4,5-dihydroisoxazol-3-yl)-1-naphthoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 8.90-8.86 (m, 1H), 8.16-8.12 (m, 1H), 7.85 (t, J=2.0 Hz, 1H), 7.79 (s, 1H), 7.70 (q, J=1.3 Hz, 1H), 7.69-7.63 (m, 2H), 7.58-7.51 (m, 2H), 4.68 (dt, J=47.3, 4.9 Hz, 2H), 4.31 (d, J=17.2 Hz, 1H), 4.21 (dt, J=24.7, 4.8 Hz, 2H), 3.96-3.87 (m, 1H), 3.26 (q, J=6.8 Hz, 1H), 1.76-1.55 (m, 4H).

### Synthesis Example 18—Synthesis of Compound 1018

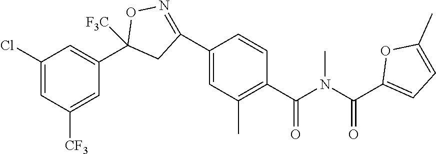 [SMILES: Cc1ccc(C(=O)N(C)C(=O)c2ccc(C3=NOC(c4cc(Cl)cc(C(F)(F)F)c4)(C(F)(F)F)C3)cc2C)o1]

Compound 1018 was obtained through the same process as in Synthesis Example 1 except that 5-methyl-2-furancarbonyl chloride was used in place of cyclopropanecarbonyl chloride, and that SM2 was used in place of SM1.

¹H NMR (500 MHz, DMSO-d₆): δ 11.31 (s, 1H), 8.09 (s, 1H), 7.98 (s, 1H), 7.86 (s, 1H), 7.65 (d, J=2.0 Hz, 1H), 7.62 (dd, J=8.0, 2.1 Hz, 1H), 7.52 (d, J=3.5 Hz, 1H), 7.49 (d, J=8.1 Hz, 1H), 6.36 (dd, J=3.5, 1.1 Hz, 1H), 4.49-4.35 (m, 2H), 2.36 (s, 3H), 2.35 (s, 3H).

### Synthesis Example 19—Synthesis of Compound 1019

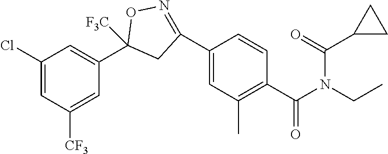 [SMILES: CCN(C(=O)c1ccc(C2=NOC(c3cc(Cl)cc(C(F)(F)F)c3)(C(F)(F)F)C2)cc1C)C(=O)C1CC1]

Compound 1019 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.80 (s, 1H), 7.73 (s, 1H), 7.67 (s, 1H), 7.53 (s, 1H), 7.52-7.49 (m, 1H), 7.29 (d, J=7.9 Hz, 1H), 4.11 (d, J=17.2 Hz, 1H), 3.88 (q, J=7.0 Hz, 2H), 3.69 (d, J=17.2 Hz, 1H), 2.40 (s, 3H), 1.74-1.66 (m, 2H), 1.16-1.14 (m, 1H), 1.02-0.99 (m, 3H), 0.72 (dd, J=7.8, 3.4 Hz, 2H).

### Synthesis Example 20—Synthesis of Compound 1020

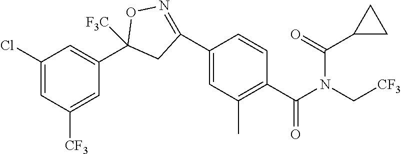 [SMILES: Cc1cc(C2=NOC(c3cc(Cl)cc(C(F)(F)F)c3)(C(F)(F)F)C2)ccc1C(=O)N(CC(F)(F)F)C(=O)C1CC1]

Compound 1020 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.80 (s, 1H), 7.74 (s, 1H), 7.68 (s, 1H), 7.58 (s, 1H), 7.54 (dd, J=7.9, 1.8 Hz, 1H), 7.37 (d, J=8.1 Hz, 1H), 4.58 (q, J=8.5 Hz, 2H), 4.12 (d, J=17.2 Hz, 1H), 3.70 (d, J=17.4 Hz, 1H), 2.46 (s, 3H), 0.93 (dt, J=4.9, 3.2 Hz, 1H), 0.88-0.84 (m, 2H), 0.68 (ddd, J=7.9, 4.5, 1.7 Hz, 2H).

### Synthesis Example 21—Synthesis of Compound 1021

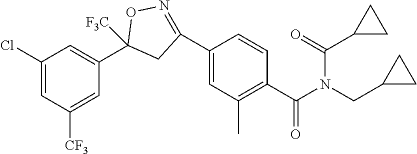 [SMILES: Cc1cc(C2=NOC(c3cc(Cl)cc(C(F)(F)F)c3)(C(F)(F)F)C2)ccc1C(=O)N(CC1CC1)C(=O)C1CC1]

Compound 1021 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.80 (s, 1H), 7.74 (s, 1H), 7.67 (s, 1H), 7.53 (s, 1H), 7.53-7.50 (m, 1H), 7.36 (d, J=7.9 Hz, 1H), 4.12 (d, J=17.2 Hz, 1H), 3.74 (d, J=7.0 Hz, 2H), 3.70 (d, J=17.4 Hz, 1H), 2.43 (s, 3H), 1.71-1.65 (m, 1H), 1.65-1.59 (m, 1H), 1.15 (dd, J=4.6, 3.1 Hz, 2H), 0.68 (h, J=4.2 Hz, 2H), 0.52-0.47 (m, 2H), 0.32-0.27 (m, 2H).

### Synthesis Example 22—Synthesis of Compound 1022

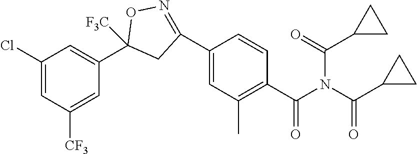 [SMILES: Cc1cc(C2=NOC(c3cc(Cl)cc(C(F)(F)F)c3)(C(F)(F)F)C2)ccc1C(=O)N(C(=O)C1CC1)C(=O)C1CC1]

Compound 1022 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.80 (s, 1H), 7.73 (s, 1H), 7.68 (t, J=1.8 Hz, 1H), 7.63-7.56 (m, 2H), 7.52 (d, J=2.0 Hz, 1H), 4.12 (d, J=17.2 Hz, 1H), 3.70 (d, J=17.2 Hz, 1H), 2.55 (s, 3H), 2.03 (ddd, J=7.8, 4.7, 3.2 Hz, 2H), 1.14 (td, J=4.4, 2.5 Hz, 4H), 1.02 (dq, J=7.2, 3.7 Hz, 4H).

### Synthesis Example 23—Synthesis of Compound 1023

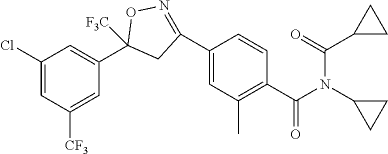 [SMILES: Cc1cc(C2=NOC(c3cc(Cl)cc(C(F)(F)F)c3)(C(F)(F)F)C2)ccc1C(=O)N(C(=O)C1CC1)C1CC1]

Compound 1023 was obtained through the same process as in Synthesis Example 15 except that 4-(5-(3-chloro-5-(trifluoromethyl)phenyl)-5-(trifluoromethyl)-4,5-dihydroisoxazol-3-yl)-2-methylbenzoic acid was used in place of 4-(5-(3,5-dichlorophenyl)-5-(trifluoromethyl)-4,5-dihydroisoxazol-3-yl)-1-naphthoic acid.

¹H NMR (500 MHz, CDCl₃): δ 7.79 (s, 1H), 7.73 (s, 1H), 7.66 (s, 1H), 7.50-7.46 (m, 2H), 7.21 (d, J=8.5 Hz, 1H), 4.10 (d, J=17.2 Hz, 1H), 3.68 (d, J=17.1 Hz, 1H), 2.83 (tt, J=6.9, 3.8 Hz, 1H), 2.35 (d, J=2.0 Hz, 3H), 1.59 (tt, J=8.1, 4.6 Hz, 1H), 1.05-1.00 (m, 4H), 0.92 (ddt, J=8.1, 6.9, 3.3 Hz, 4H).

### Synthesis Example 24—Synthesis of Compound 1024

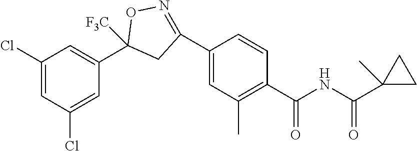 [SMILES: Cc1cc(C2=NOC(c3cc(Cl)cc(Cl)c3)(C(F)(F)F)C2)ccc1C(=O)NC(=O)C1(C)CC1]

Compound 1024 was obtained through the same process as in Synthesis Example 1 except that 1-methylcyclopropanecarbonyl chloride was used in place of cyclopropanecarbonyl chloride.

¹H NMR (500 MHz, CDCl₃): δ 8.45 (s, 1H), 7.55-7.46 (m, 4H), 7.41 (q, J=2.1 Hz, 1H), 7.33 (s, 1H), 4.05 (d, J=17.1 Hz, 1H), 3.66 (d, J=17.2 Hz, 1H), 2.39 (s, 3H), 1.42 (s, 3H), 1.27 (s, 2H), 0.76 (s, 1H), 0.74-0.71 (m, 1H).

### Synthesis Example 25—Synthesis of Compound 1025

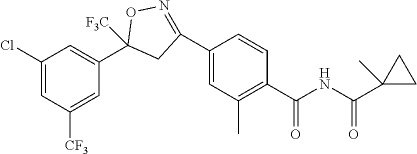 [SMILES: Cc1cc(C2=NOC(c3cc(Cl)cc(C(F)(F)F)c3)(C(F)(F)F)C2)ccc1C(=O)NC(=O)C1(C)CC1]

Compound 1025 was obtained through the same process as in Synthesis Example 1 except that 1-methylcyclopropanecarbonyl chloride was used in place of cyclopropanecarbonyl chloride, and that SM2 was used in place of SM1.

¹H NMR (500 MHz, CDCl₃): δ 8.46 (s, 1H), 7.79 (s, 1H), 7.73 (s, 1H), 7.67 (s, TH), 7.54-7.49 (m, 2H), 7.32 (d, J=8.4 Hz, TH), 4.10 (d, J=17.1 Hz, TH), 3.68 (d, J=17.0 Hz, 1H), 2.39 (s, 3H), 1.41 (s, 3H), 1.25 (q, J=4.0 Hz, 2H), 0.78-0.74 (m, 2H).

### Synthesis Example 26—Synthesis of Compound 1026

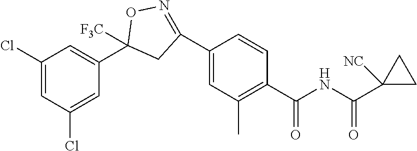 [SMILES: Cc1cc(C2=NOC(c3cc(Cl)cc(Cl)c3)(C(F)(F)F)C2)ccc1C(=O)NC(=O)C1(C#N)CC1]

Compound 1026 was obtained through the same process as in Synthesis Example 1 except that 1-cyanocyclopropanecarbonyl chloride was used in place of cyclopropanecarbonyl chloride.

¹H NMR (500 MHz, CDCl₃): δ 8.96 (s, 1H), 7.56 (dd, J=7.6, 0.7 Hz, 2H), 7.50-7.45 (m, 3H), 7.41 (t, J=1.8 Hz, 1H), 4.06 (d, J=17.1 Hz, 1H), 3.68 (d, J=17.2 Hz, 1H), 2.48 (s, 3H), 1.81-1.76 (m, 2H), 1.72-1.67 (m, 2H).

### Synthesis Example 27—Synthesis of Compound 1027

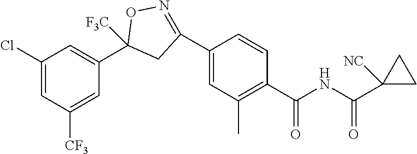 [SMILES: Cc1cc(C2=NOC(c3cc(Cl)cc(C(F)(F)F)c3)(C(F)(F)F)C2)ccc1C(=O)NC(=O)C1(C#N)CC1]

Compound 1027 was obtained through the same process as in Synthesis Example 1 except that 1-cyanocyclopropanecarbonyl chloride was used in place of cyclopropanecarbonyl chloride, and that SM2 was used in placr of SM1.

¹H NMR (500 MHz, DMSO-d₆): δ 11.43 (s, 1H), 8.08 (s, 1H), 7.98 (s, 1H), 7.85 (s, 1H), 7.69-7.61 (m, 2H), 7.56 (d, J=8.1 Hz, 1H), 4.46 (d, J=18.3 Hz, 1H), 4.37 (d, J=18.3 Hz, 1H), 2.40 (s, 3H), 1.73 (dd, J=3.8, 1.7 Hz, 4H).

### Synthesis Example 28—Synthesis of Compound 1028

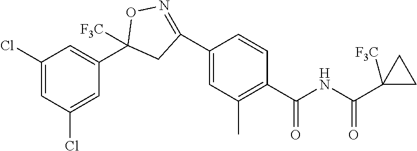 [SMILES: Cc1cc(C2=NOC(c3cc(Cl)cc(Cl)c3)(C(F)(F)F)C2)ccc1C(=O)NC(=O)C1(C(F)(F)F)CC1]

Compound 1028 was obtained through the same process as in Synthesis Example 1 except that 1-(trifluoromethyl)cyclopropanecarbonyl chloride was used in place of cyclopropanecarbonyl chloride.

¹H NMR (500 MHz, CDCl₃): δ 8.66 (s, 1H), 7.57-7.53 (m, 2H), 7.48 (d, J=1.8 Hz, 2H), 7.42-7.38 (m, 2H), 4.06 (d, J=17.1 Hz, 1H), 3.67 (d, J=17.2 Hz, 1H), 2.45 (s, 3H), 1.54-1.49 (m, 2H), 1.39-1.36 (m, 2H).

### Synthesis Example 29—Synthesis of Compound 1029

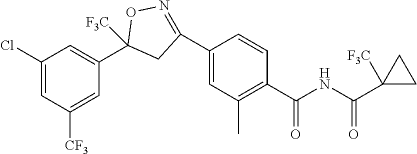 [SMILES: Cc1cc(C2=NOC(c3cc(Cl)cc(C(F)(F)F)c3)(C(F)(F)F)C2)ccc1C(=O)NC(=O)C1(C(F)(F)F)CC1]

Compound 1029 was obtained through the same process as in Synthesis Example 1 except that 1-(trifluoromethyl)cyclopropanecarbonyl chloride was used in place of cyclopropanecarbonyl chloride, and that SM2 was used in place of SM1.

¹H NMR (500 MHz, DMSO-d₆): δ 10.95 (s, 1H), 8.08 (s, 1H), 7.97 (s, 1H), 7.85 (s, 1H), 7.66-7.58 (m, 2H), 7.44 (d, J=8.1 Hz, 1H), 4.45 (d, J=18.5 Hz, 1H), 4.36 (d, J=18.3 Hz, 1H), 2.33 (s, 3H), 1.61 (d, J=1.7 Hz, 2H), 1.37-1.33 (m, 2H).

### Synthesis Example 30—Synthesis of Compound 1030

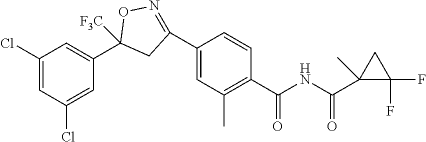 [SMILES: Cc1cc(C2=NOC(c3cc(Cl)cc(Cl)c3)(C(F)(F)F)C2)ccc1C(=O)NC(=O)C1(C)CC1(F)F]

Compound 1030 was obtained through the same process as in Synthesis Example 1 except that 2,2-difluoro-1-methylcyclopropanecarbonyl chloride was used in place of cyclopropanecarbonyl chloride.

¹H NMR (500 MHz, CDCl₃): δ 8.26 (s, 1H), 7.56-7.52 (m, 2H), 7.49 (d, J=2.0 Hz, 2H), 7.44-7.39 (m, 2H), 4.06 (dd, J=17.1, 0.8 Hz, 1H), 3.68 (d, J=17.2 Hz, 1H), 2.46 (s, 3H), 2.16-2.08 (m, 1H), 1.58 (dd, J=2.8, 1.6 Hz, 3H), 1.36 (ddd, J=11.1, 8.4, 5.3 Hz, 1H).

### Synthesis Example 31—Synthesis of Compound 1031

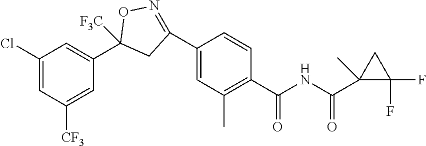 [SMILES: Cc1cc(C2=NOC(c3cc(Cl)cc(C(F)(F)F)c3)(C(F)(F)F)C2)ccc1C(=O)NC(=O)C1(C)CC1(F)F]

Compound 1031 was obtained through the same process as in Synthesis Example 1 except that 2,2-difluoro-1-methylcyclopropanecarbonyl chloride was used in place of cyclopropanecarbonyl chloride, and that SM2 was used in place of SM1.

¹H NMR (500 MHz, CDCl₃): δ 8.22 (s, 1H), 7.80 (s, 1H), 7.73 (s, 1H), 7.68 (s, 1H), 7.59-7.54 (m, 2H), 7.42 (d, J=8.5 Hz, 1H), 4.12 (dd, J=17.2, 0.9 Hz, 1H), 3.70 (d, J=17.2 Hz, 1H), 2.46 (s, 3H), 1.58 (dd, J=2.8, 1.6 Hz, 3H), 1.45 (dd, J=2.9, 2.0 Hz, 1H), 1.38-1.34 (m, 1H).

### Synthesis Example 32—Synthesis of Compound 1032

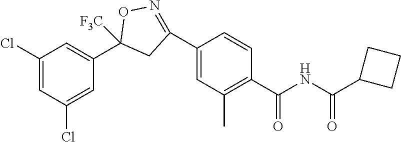 [SMILES: Cc1cc(C2=NOC(c3cc(Cl)cc(Cl)c3)(C(F)(F)F)C2)ccc1C(=O)NC(=O)C1CCC1]

Compound 1032 was obtained through the same process as in Synthesis Example 1 except that cyclobutanecarbonyl chloridewas used in place of cyclopropanecarbonyl chloride.

¹H NMR (500 MHz, CDCl₃): δ 8.28 (s, 1H), 7.56-7.51 (m, 2H), 7.49 (d, J=2.0 Hz, 2H), 7.45 (d, J=7.9 Hz, 1H), 7.41 (t, J=1.9 Hz, 1H), 4.06 (d, J=17.1 Hz, 1H), 3.68 (d, J=17.2 Hz, 1H), 3.16 (pd, J=8.5, 1.1 Hz, 1H), 2.46 (s, 3H), 2.24-2.19 (m, 2H), 2.06-1.95 (m, 2H), 1.94-1.87 (m, 2H).

### Synthesis Example 33—Synthesis of Compound 1033

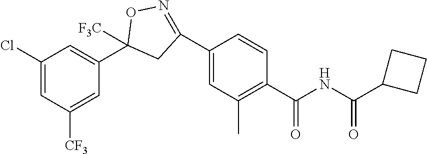 [SMILES: Cc1cc(C2=NOC(c3cc(Cl)cc(C(F)(F)F)c3)(C(F)(F)F)C2)ccc1C(=O)NC(=O)C1CCC1]

Compound 1033 was obtained through the same process as in Synthesis Example 1 except that cyclobutanecarbonyl chloride was used in place of cyclopropanecarbonyl chloride, and that SM2 was used in place of SM1.

¹H NMR (500 MHz, CDCl₃): δ 8.05 (s, 1H), 7.80 (s, 1H), 7.73 (s, 1H), 7.67 (s, 1H), 7.58-7.52 (m, 2H), 7.44 (d, J=7.9 Hz, 1H), 4.12 (d, J=17.2 Hz, 1H), 3.87 (pd, J=8.5, 1.2 Hz, 1H), 3.70 (d, J=17.2 Hz, 1H), 2.46 (s, 3H), 2.39-2.27 (m, 4H), 2.10-1.96 (m, 1H), 1.95-1.83 (m, 1H).

### Synthesis Example 34—Synthesis of Compound 1034

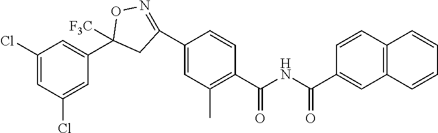 [SMILES: Cc1cc(C2=NOC(c3cc(Cl)cc(Cl)c3)(C(F)(F)F)C2)ccc1C(=O)NC(=O)c1ccc2ccccc2c1]

Compound 1034 was obtained through the same process as in Synthesis Example 1 except that 2-naphthoyl chloride was used in place of cyclopropanecarbonyl chloride.

¹H NMR (500 MHz, CDCl₃): δ 9.03 (s, 1H), 8.42 (d, J=1.8 Hz, 1H), 7.98 (dd, is J=8.3, 1.9 Hz, 2H), 7.91 (ddd, J=20.1, 8.3, 1.6 Hz, 2H), 7.85 (d, J=1.9 Hz, 1H), 7.79 (s, 1H), 7.75-7.68 (m, 1H), 7.68-7.64 (m, 1H), 7.64-7.61 (m, 2H), 7.61 (d, J=1.1 Hz, 1H), 7.54 (d, J=7.9 Hz, 1H), 4.22-4.12 (m, 1H), 3.76 (d, J=17.2 Hz, 1H), 2.53 (s, 3H).

### Synthesis Example 35—Synthesis of Compound 1035

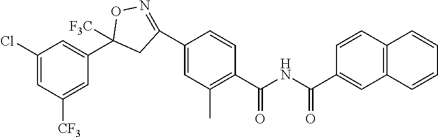 [SMILES: Cc1cc(C2=NOC(c3cc(Cl)cc(C(F)(F)F)c3)(C(F)(F)F)C2)ccc1C(=O)NC(=O)c1ccc2ccccc2c1]

Compound 1035 was obtained through the same process as in Synthesis Example 1 except that 2-naphthoyl chloride was used in place of cyclopropanecarbonyl chloride, and that SM2 was used in place of SM1.

¹H NMR (500 MHz, CDCl₃): δ 9.03 (s, 1H), 8.42 (d, J=1.8 Hz, 1H), 7.98 (dd, J=8.3, 1.9 Hz, 2H), 7.91 (ddd, J=20.1, 8.3, 1.6 Hz, 2H), 7.85 (d, J=1.9 Hz, 1H), 7.79 (s, 1H), 7.75-7.68 (m, 1H), 7.68-7.64 (m, 1H), 7.64-7.61 (m, 2H), 7.61 (d, J=1.1 Hz, 1H), 7.54 (d, J=7.9 Hz, 1H), 4.22-4.12 (m, 1H), 3.76 (d, J=17.2 Hz, 1H), 2.53 (s, 3H).

### Synthesis Example 36—Synthesis of Compound 1036

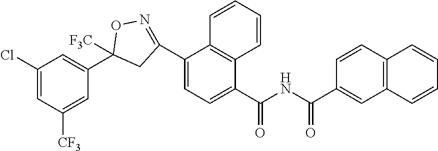 [SMILES: O=C(NC(=O)c1ccc(C2=NOC(c3cc(Cl)cc(C(F)(F)F)c3)(C(F)(F)F)C2)c2ccccc12)c1ccc2ccccc2c1]

Compound 1036 was obtained through the same process as in Synthesis Example 1 except that 2-naphthoyl chloride was used in place of cyclopropanecarbonyl chloride, and that SM3 was used in place of SM1.

¹H NMR (500 MHz, CDCl₃): δ 9.27 (s, 1H), 8.96-8.91 (m, 1H), 8.44-8.40 (m, 1H), 8.26 (dt, J=8.3, 1.0 Hz, 1H), 7.96-7.93 (m, 2H), 7.93-7.86 (m, 3H), 7.84 (s, is 1H), 7.76-7.74 (m, 1H), 7.73-7.69 (m, 2H), 7.68-7.64 (m, 2H), 7.63-7.58 (m, 2H), 4.36 (d, J=17.2 Hz, 1H), 3.96 (d, J=17.2 Hz, 1H).

### Synthesis Example 37—Synthesis of Compound 1037

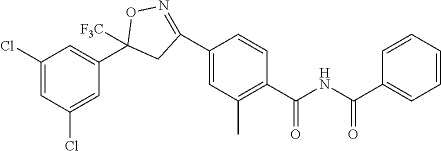 [SMILES: Cc1cc(C2=NOC(c3cc(Cl)cc(Cl)c3)(C(F)(F)F)C2)ccc1C(=O)NC(=O)c1ccccc1]

Compound 1037 was obtained through the same process as in Synthesis Example 1 except that benzoyl chloride was used in place of cyclopropanecarbonyl chloride.

¹H NMR (500 MHz, DMSO-d₆): δ 11.58 (s, 1H), 8.14-8.09 (m, 1H), 7.96-7.88 (m, 4H), 7.57-7.50 (m, 6H), 4.38 (s, 1H), 4.33 (s, 1H), 2.39 (s, 3H).

### Synthesis Example 38—Synthesis of Compound 1038

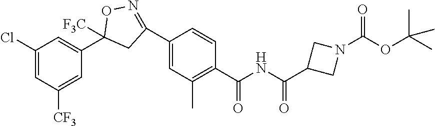 [SMILES: Cc1cc(C2=NOC(c3cc(Cl)cc(C(F)(F)F)c3)(C(F)(F)F)C2)ccc1C(=O)NC(=O)C1CN(C(=O)OC(C)(C)C)C1]

Compound 1038 was obtained through the same process as in Synthesis Example 1 except that tert-butyl 3-(chlorocarbonyl)azetidine-1-carboxylate was used in place of cyclopropanecarbonyl chloride.

¹H NMR (500 MHz, CDCl₃): δ 8.35 (s, 1H), 7.58-7.54 (m, 2H), 7.49 (q, J=3.1 Hz, 3H), 7.41 (t, J=1.8 Hz, 1H), 4.26-4.12 (m, 4H), 4.09-4.04 (m, 2H), 3.69 (d, J=17.2 Hz, 1H), 2.48 (s, 3H), 1.42 (s, 9H).

### Synthesis Example 39—Synthesis of Compound 1039

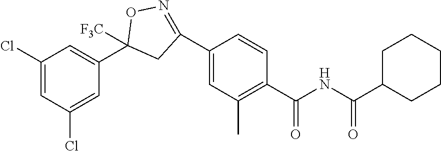 [SMILES: Cc1cc(C2=NOC(c3cc(Cl)cc(Cl)c3)(C(F)(F)F)C2)ccc1C(=O)NC(=O)C1CCCCC1]

Compound 1039 was obtained through the same process as in Synthesis Example 1 except that cyclohexanecarbonyl chloride was used in place of cyclopropanecarbonyl chloride.

¹H NMR (500 MHz, CDCl₃): δ 8.06 (s, 1H), 7.56-7.51 (m, 2H), 7.49 (d, J=1.8 Hz, 2H), 7.45-7.39 (m, 2H), 4.06 (d, J=17.2 Hz, 1H), 3.67 (d, J=17.2 Hz, 1H), 3.03 (tt, J=11.4, 3.5 Hz, 1H), 2.46 (s, 3H), 1.99-1.92 (m, 2H), 1.81 (dt, J=13.4, 3.5 Hz, 2H), 1.70 (dtd, J=13.0, 3.4, 1.8 Hz, 1H), 1.46 (qd, J=12.4, 3.1 Hz, 2H), 1.35 (qt, J=12.7, 3.1 Hz, 2H), 1.24 (tt, J=12.2, 3.4 Hz, 1H).

### Synthesis Example 40—Synthesis of Compound 1040

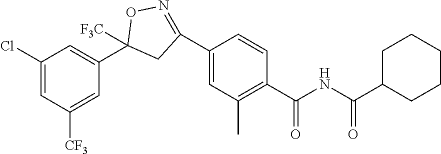 [SMILES: Cc1cc(C2=NOC(c3cc(Cl)cc(C(F)(F)F)c3)(C(F)(F)F)C2)ccc1C(=O)NC(=O)C1CCCCC1]

Compound 1040 was obtained through the same process as in Synthesis Example 1 except that cyclohexanecarbonyl chloride was used in place of cyclopropanecarbonyl chloride, and that SM2 was used in place of SM1.

¹H NMR (500 MHz, CDCl₃): δ 8.13 (s, 1H), 7.75 (d, J=1.8 Hz, 1H), 7.68 (s, 1H), 7.67-7.60 (m, 1H), 7.49 (d, J=9.4 Hz, 2H), 7.38 (d, J=7.8 Hz, 1H), 4.07 (d, J=17.2 Hz, 1H), 3.65 (d, J=17.2 Hz, 1H), 2.97 (s, 1H), 2.41 (s, 3H), 1.76 (dt, J=13.4, 3.7 Hz, 2H), 1.33-1.11 (m, 8H).

### Synthesis Example 41—Synthesis of Compound 1041

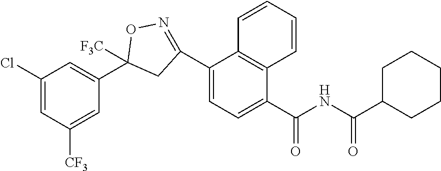 [SMILES: O=C(NC(=O)C1CCCCC1)c1ccc(C2=NOC(c3cc(Cl)cc(C(F)(F)F)c3)(C(F)(F)F)C2)c2ccccc12]

Compound 1041 was obtained through the same process as in Synthesis Example 1 except that cyclohexanecarbonyl chloride was used instead of cyclopropanecarbonyl chloride, and that SM3 was used in place of SM1.

¹H NMR (500 MHz, CDCl₃): δ 8.93-8.87 (m, 1H), 8.67 (s, 1H), 8.28-8.23 (m, 1H), 7.89 (d, J=1.9 Hz, 1H), 7.83 (s, 1H), 7.74 (t, J=1.9 Hz, 1H), 7.74-7.64 (m, 3H), 7.58 (d, J=7.5 Hz, 1H), 4.35 (d, J=17.2 Hz, 1H), 4.04-3.92 (m, 1H), 3.20 (tt, J=11.4, 3.4 Hz, 1H), 1.78-1.77 (m, 2H), 1.37-1.29 (m, 8H).

### Synthesis Example 42—Synthesis of Compound 1042

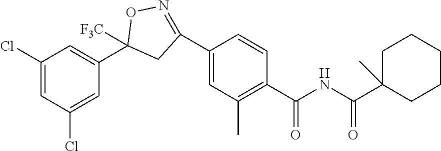 [SMILES: Cc1cc(C2=NOC(c3cc(Cl)cc(Cl)c3)(C(F)(F)F)C2)ccc1C(=O)NC(=O)C1(C)CCCCC1]

Compound 1042 was obtained through the same process as in Synthesis Example 1 except that 1-methylcyclohexanecarbonyl chloride was used in place of cyclopropanecarbonyl chloride.

¹H NMR (500 MHz, CDCl₃): δ 8.44 (s, 1H), 7.52 (dt, J=3.8, 2.1 Hz, 2H), 7.48 (d, J=2.0 Hz, 2H), 7.41 (t, J=1.8 Hz, 1H), 7.29 (d, J=8.5 Hz, 1H), 4.05 (d, J=17.1 Hz, 1H), 3.67 (d, J=17.4 Hz, 1H), 2.39 (s, 3H), 2.05-1.98 (m, 4H), 1.90 (dd, J=11.0, 7.0 Hz, 2H), 1.50 (dd, J=6.1, 2.9 Hz, 2H), 1.37 (t, J=1.8 Hz, 2H), 1.21 (s, 3H).

### Synthesis Example 43—Synthesis of Compound 1043

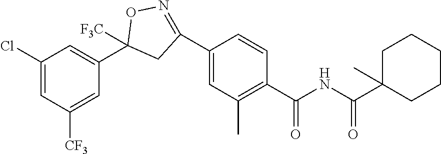 [SMILES: Cc1cc(C2=NOC(c3cc(Cl)cc(C(F)(F)F)c3)(C(F)(F)F)C2)ccc1C(=O)NC(=O)C1(C)CCCCC1]

Compound 1043 was obtained through the same process as in Synthesis Example 1 except that 1-methylcyclohexanecarbonyl chloride was used in place of cyclopropanecarbonyl chloride, and that SM2 was used in place of SM1.

¹H NMR (500 MHz, CDCl₃): δ 8.45 (s, 1H), 7.84 (d, J=1.9 Hz, 1H), 7.78 (s, is 1H), 7.72 (q, J=1.7, 1.2 Hz, 1H), 7.60-7.55 (m, 2H), 7.34 (d, J=8.5 Hz, 1H), 4.16 (d, J=17.2 Hz, 1H), 3.74 (d, J=17.2 Hz, 1H), 2.43 (s, 3H), 1.95 (t, J=5.6 Hz, 2H), 1.65-1.60 (m, 2H), 1.47-1.37 (m, 6H), 1.25 (s, 3H).

### Synthesis Example 44—Synthesis of Compound 1044

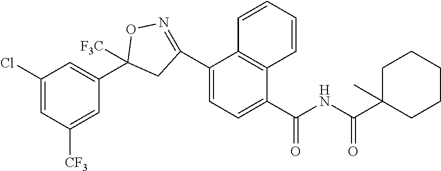 [SMILES: CC1(C(=O)NC(=O)c2ccc(C3=NOC(c4cc(Cl)cc(C(F)(F)F)c4)(C(F)(F)F)C3)c3ccccc23)CCCCC1]

Compound 1044 was obtained through the same process as in Synthesis Example 1 except that 1-methylcyclohexanecarbonyl chloride was used in place of cyclopropanecarbonyl chloride, and that SM3 was used in place of SM1.

¹H NMR (500 MHz, CDCl₃): δ 8.97-8.91 (m, 1H), 8.56 (s, 1H), 8.09 (dt, J=8.4, 1.0 Hz, 1H), 7.89 (d, J=1.9 Hz, 1H), 7.83 (s, 1H), 7.76-7.72 (m, 1H), 7.72-7.67 (m, 1H), 7.67-7.64 (m, 1H), 7.64-7.57 (m, 1H), 7.54 (d, J=7.4 Hz, 1H), 4.36 (d, J=17.1 Hz, 1H), 3.96 (d, J=17.1 Hz, 1H), 1.93 (dt, J=9.1, 3.9 Hz, 2H), 1.61 (dd, J=9.5, 3.4 Hz, 2H), 1.51-1.33 (m, 6H), 1.26 (s, 3H).

### Synthesis Example 45—Synthesis of Compound 1045

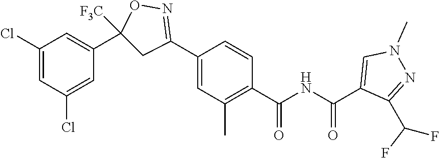 [SMILES: Cc1cc(C2=NOC(c3cc(Cl)cc(Cl)c3)(C(F)(F)F)C2)ccc1C(=O)NC(=O)c1cn(C)nc1C(F)F]

Compound 1045 was obtained through the same process as in Synthesis Example 1 except that 3-difluoromethyl-1-methylpyrazole-4-carbonyl chloride was used in place of cyclopropanecarbonyl chloride.

¹H NMR (500 MHz, DMSO-d₆): δ 11.36 (s, 1H), 8.60 (t, J=1.3 Hz, 1H), 7.81 (t, J=1.9 Hz, 1H), 7.68-7.59 (m, 4H), 7.49 (d, J=7.9 Hz, 1H), 7.27-7.02 (m, 1H), 4.40 (d, J=18.3 Hz, 1H), 4.31 (d, J=18.5 Hz, 1H), 3.94 (s, 3H), 2.36 (s, 3H).

### Synthesis Example 46—Synthesis of Compound 1046

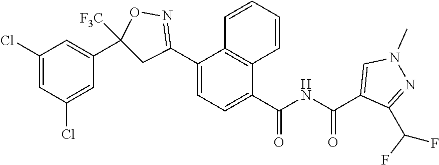 [SMILES: Cn1cc(C(=O)NC(=O)c2ccc(C3=NOC(c4cc(Cl)cc(Cl)c4)(C(F)(F)F)C3)c3ccccc23)c(C(F)F)n1]

Compound 1046 was obtained through the same process as in Synthesis Example 1 except that 3-difluoromethyl-1-methylpyrazole-4-carbonyl chloride was used in place of cyclopropanecarbonyl chloride, and that SM10 was used in place of SM1.

¹H NMR (500 MHz, DMSO-d₆): δ 11.63 (s, 1H), 8.82 (ddd, J=8.7, 1.4, 0.8 Hz, 1H), 8.62 (t, J=1.2 Hz, 1H), 8.10-8.05 (m, 1H), 7.92 (d, J=7.6 Hz, 1H), 7.83 (t, J=1.9 Hz, 1H), 7.76-7.66 (m, 5H), 7.25-6.96 (m, 1H), 4.62-4.50 (m, 2H), 3.94 (s, 3H).

### Synthesis Example 47—Synthesis of Compound 1047

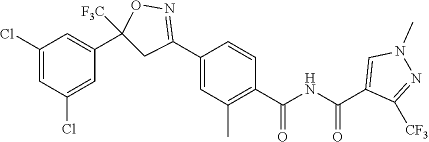 [SMILES: Cc1cc(C2=NOC(c3cc(Cl)cc(Cl)c3)(C(F)(F)F)C2)ccc1C(=O)NC(=O)c1cn(C)nc1C(F)(F)F]

Compound 1047 was obtained through the same process as in Synthesis Example 1 except that 1-methyl-3-(trifluoromethyl)-1H-pyrazole-4-carbonyl chloride was used in place of cyclopropanecarbonyl chloride.

¹H NMR (500 MHz, DMSO-d₆): δ 11.64 (s, 1H), 8.82 (ddd, J=8.6, 1.4, 0.7 Hz, 1H), 8.63 (d, J=1.3 Hz, 1H), 8.08 (ddd, J=8.3, 1.5, 0.7 Hz, 1H), 7.93 (d, J=7.5 Hz, 1H), 7.84 (t, J=1.9 Hz, 1H), 7.78-7.65 (m, 5H), 4.57 (d, J=6.2 Hz, 2H), 3.95 (s, 3H).

### Synthesis Example 48—Synthesis of Compound 1048

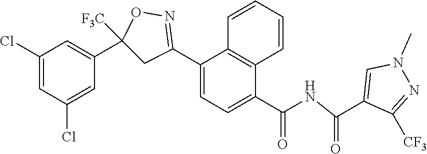 [SMILES: Cn1cc(C(=O)NC(=O)c2ccc(C3=NOC(c4cc(Cl)cc(Cl)c4)(C(F)(F)F)C3)c3ccccc23)c(C(F)(F)F)n1]

Compound 1048 was obtained through the same process as in Synthesis Example 1 except that 1-methyl-3-(trifluoromethyl)-1H-pyrazole-4-carbonyl chloride was used in place of cyclopropanecarbonyl chloride, and that SM10 was used in place of SM1.

¹H NMR (500 MHz, DMSO-d₆): δ 11.69 (s, 1H), 8.82 (dt, J=8.6, 1.0 Hz, 1H), 8.70 (d, J=1.1 Hz, 1H), 8.14-8.08 (m, 1H), 7.93 (d, J=7.5 Hz, 1H), 7.84 (t, J=1.9 Hz, 1H), 7.81-7.66 (m, 5H), 4.57 (d, J=6.2 Hz, 2H), 3.97 (s, 3H).

### Synthesis Example 49—Synthesis of Compound 1049

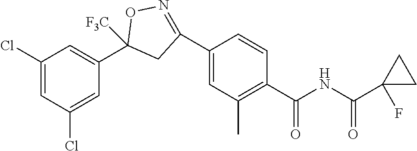 [SMILES: Cc1cc(C2=NOC(c3cc(Cl)cc(Cl)c3)(C(F)(F)F)C2)ccc1C(=O)NC(=O)C1(F)CC1]

Compound 1049 was obtained through the same process as in Synthesis Example 1 except that 1-fluorocyclopropanecarbonyl chloride was used in place of cyclopropanecarbonyl chloride.

¹H NMR (300 Hz, CDCl₃): δ 9.05 (d, J=4.1 Hz, 1H), 7.55 (d, J=5.6 Hz, 2H), 7.48 (d, J=1.4 Hz, 2H), 7.44 (d, J=8.5 Hz, 1H), 7.41 (t, J=1.8 Hz, 1H), 4.06 (d, J=15.7 Hz, 1H), 3.68 (d, J=17.2 Hz, 1H), 2.46 (s, 3H), 1.47 (s, 2H), 1.42 (q, J=3.3 Hz, 2H).

### Synthesis Example 50—Synthesis of Compound 1050

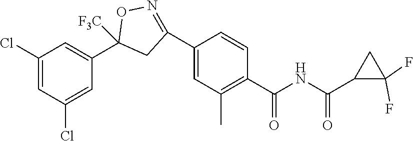 [SMILES: Cc1cc(C2=NOC(c3cc(Cl)cc(Cl)c3)(C(F)(F)F)C2)ccc1C(=O)NC(=O)C1CC1(F)F]

Compound 1050 was obtained through the same process as in Synthesis Example 1 except that 2,2-difluorocyclopropanecarbonyl chloride was used in place of cyclopropanecarbonyl chloride.

¹H NMR (300 Hz, CDCl₃): δ 8.37 (s, 1H), 7.57 (d, J=6.8 Hz, 2H), 7.53 (s, 1H), 7.51-7.47 (m, 2H), 7.42 (t, J=1.8 Hz, 1H), 4.07 (d, J=17.3 Hz, 1H), 3.79-3.64 (m, 2H), 2.52 (s, 3H), 2.35-2.22 (m, 1H), 1.89-1.76 (m, 1H).

### Synthesis Example 51—Synthesis of Compound 1051

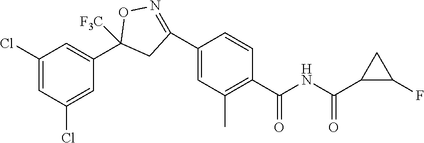 [SMILES: Cc1cc(C2=NOC(c3cc(Cl)cc(Cl)c3)(C(F)(F)F)C2)ccc1C(=O)NC(=O)C1CC1F]

Compound 1051 was obtained through the same process as in Synthesis Example 1 except that 2-fluorocyclopropanecarbonyl chloride was used in place of xyclopropanecarbonyl chloride.

¹H NMR (300 MHz, CDCl₃): δ 8.20 (s, 1H), 7.56 (d, J=6.7 Hz, 2H), 7.52-7.48 (m, 3H), 7.42 (t, J=1.8 Hz, 1H), 5.05-4.78 (m, 1H), 4.07 (d, J=17.3 Hz, 1H), 3.68 (d, J=17.1 Hz, 1H), 3.50-3.36 (m, 1H), 2.52 (s, 3H), 1.68 (ddt, J=13.6, 6.8, 3.2 Hz, 2H).

### Synthesis Example 52—Synthesis of Compound 1052

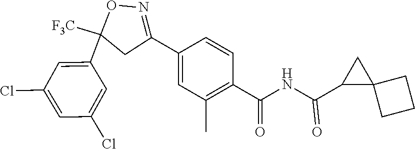 [SMILES: Cc1cc(C2=NOC(c3cc(Cl)cc(Cl)c3)(C(F)(F)F)C2)ccc1C(=O)NC(=O)C1CC12CCC2]

Compound 1052 was obtained through the same process as in Synthesis Example 1 except that spiro[2.3]hexane-1-carbonyl chloride was used in place of cyclopropanecarbonyl chloride.

¹H NMR (300 Hz, CDCl₃): δ 8.14 (s, 1H), 7.54 (d, J=7.3 Hz, 2H), 7.49 (d, J=4.2 Hz, 3H), 7.41 (t, J=1.8 Hz, 1H), 4.07 (d, J=17.2 Hz, 1H), 3.68 (d, J=17.0 Hz, is 1H), 2.72 (dd, J=8.0, 5.5 Hz, 1H), 2.51 (s, 3H), 2.41-2.33 (m, 1H), 2.24-2.12 (m, 3H), 2.12-2.01 (m, 2H), 1.40 (t, J=4.9 Hz, 1H), 1.19 (dd, J=8.1, 4.4 Hz, 1H).

### Synthesis Example 53—Synthesis of Compound 1053

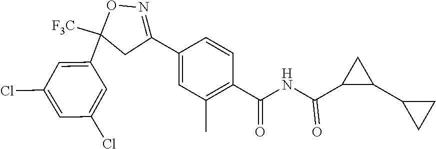 [SMILES: Cc1cc(C2=NOC(c3cc(Cl)cc(Cl)c3)(C(F)(F)F)C2)ccc1C(=O)NC(=O)C1CC1C1CC1]

Compound 1053 was obtained through the same process as in Synthesis Example 1 except that [1,1′-bicyclopropyl]-2-carbonyl chloride was used in place of cyclopropanecarbonyl chloride.

¹H NMR (300 Hz, CDCl₃): δ 9.33 (s, 1H), 7.56-7.47 (m, 4H), 7.41 (t, J=1.8 Hz, 1H), 7.37 (d, J=8.5 Hz, 1H), 4.06 (d, J=17.2 Hz, 1H), 3.66 (d, J=17.3 Hz, 1H), 2.42 (s, 3H), 1.35 (ddd, J=13.1, 7.9, 5.0 Hz, 1H), 1.13 (q, J=4.0 Hz, 2H), 0.73-0.62 (m, 4H), 0.27 (q, J=5.1 Hz, 2H).

### Synthesis Example 54—Synthesis of Compound 1054

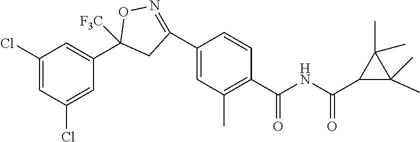 [SMILES: Cc1cc(C2=NOC(c3cc(Cl)cc(Cl)c3)(C(F)(F)F)C2)ccc1C(=O)NC(=O)C1C(C)(C)C1(C)C]

Compound 1054 was obtained through the same process as in Synthesis Example 1 except that 2,2,3,3-tetramethylcyclopropanecarbonyl chloride was used in place of cyclopropanecarbonyl chloride.

¹H NMR (500 MHz, CDCl₃): δ 8.40 (s, 1H), 7.54-7.50 (m, 2H), 7.49 (d, J=1.8 Hz, 2H), 7.45 (d, J=7.9 Hz, 1H), 7.41 (t, J=1.8 Hz, 1H), 4.06 (d, J=17.2 Hz, 1H), 3.67 (d, J=17.4 Hz, 1H), 2.47 (s, 3H), 1.26 (d, J=4.9 Hz, 13H).

### Synthesis Example 55—Synthesis of Compound 1055

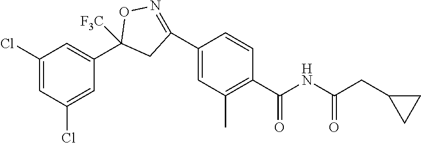 [SMILES: Cc1cc(C2=NOC(c3cc(Cl)cc(Cl)c3)(C(F)(F)F)C2)ccc1C(=O)NC(=O)CC1CC1]

Compound 1055 was obtained through the same process as in Synthesis Example 1 except that Cyclopropaneacetyl chloride was used in place of cyclopropanecarbonyl chloride.

¹H NMR (500 MHz, CDCl₃): δ 8.23 (s, 1H), 7.57-7.52 (m, 2H), 7.49 (d, J=2.0 Hz, 2H), 7.46 (d, J=7.9 Hz, 1H), 7.41 (t, J=1.8 Hz, 1H), 4.06 (d, J=17.2 Hz, 1H), 3.68 (d, J=17.4 Hz, 1H), 2.79 (d, J=7.0 Hz, 2H), 2.48 (s, 3H), 0.86 (t, J=7.0 Hz, 1H), 0.65-0.58 (m, 2H), 0.26-0.21 (m, 2H).

### Synthesis Example 56—Synthesis of Compound 1056

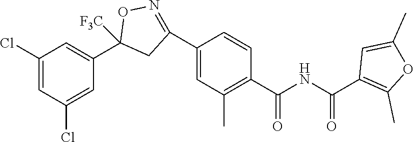 [SMILES: Cc1cc(C(=O)NC(=O)c2ccc(C3=NOC(c4cc(Cl)cc(Cl)c4)(C(F)(F)F)C3)cc2C)c(C)o1]

Compound 1056 was obtained through the same process as in Synthesis Example 1 except that 2,5-dimethyl-3-furancarbonyl chloride was used in place of cyclopropanecarbonyl chloride.

¹H NMR (500 MHz, DMSO-d₆): δ 11.03 (s, 1H), 7.81 (t, J=1.8 Hz, 1H), 7.63 (t, J=1.8 Hz, 3H), 7.60 (dd, J=7.9, 1.8 Hz, 1H), 7.44 (d, J=8.1 Hz, 1H), 6.66 (d, J=1.4 Hz, 1H), 4.43-4.35 (m, 1H), 4.31 (d, J=18.3 Hz, 1H), 2.41 (s, 3H), 2.34 (s, 3H), 2.23 (s, 3H).

### Synthesis Example 57—Synthesis of Compound 1057

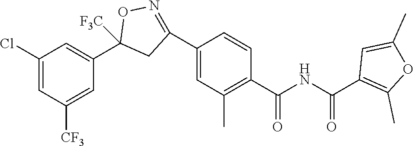 [SMILES: Cc1cc(C(=O)NC(=O)c2ccc(C3=NOC(c4cc(Cl)cc(C(F)(F)F)c4)(C(F)(F)F)C3)cc2C)c(C)o1]

Compound 1057 was obtained through the same process as in Synthesis Example 1 except that 2,5-dimethyl-3-furancarbonyl chloride was used in place of cyclopropanecarbonyl chloride, and that SM2 was used in place of SM1.

¹H NMR (500 MHz, DMSO-d₆): δ 11.03 (s, 1H), 8.09 (t, J=2.0 Hz, 1H), 7.98 (t, J=1.9 Hz, 1H), 7.86 (s, 1H), 7.64 (d, J=2.0 Hz, 1H), 7.61 (dd, J=7.9, 1.9 Hz, 1H), 7.45 (d, J=7.9 Hz, 1H), 6.66 (d, J=1.2 Hz, 1H), 4.45 (d, J=18.3 Hz, 1H), 4.38 (d, J=18.3 Hz, 1H), 2.41 (s, 3H), 2.34 (s, 3H), 2.23 (s, 3H).

### Synthesis Example 58—Synthesis of Compound 1058

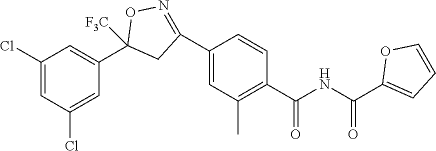 [SMILES: Cc1cc(C2=NOC(c3cc(Cl)cc(Cl)c3)(C(F)(F)F)C2)ccc1C(=O)NC(=O)c1ccco1]

Compound 1058 was obtained through the same process as in Synthesis Example 1 except that 2-furancarbonyl chloride was used in place of cyclopropanecarbonyl chloride.

¹H NMR (500 MHz, DMSO-d₆): δ 11.44 (s, 1H), 8.00 (d, J=2.4 Hz, 1H), 7.81 (t, J=1.9 Hz, 1H), 7.68-7.59 (m, 5H), 7.51 (d, J=8.1 Hz, 1H), 6.72 (dd, J=3.7, 1.8 Hz, 1H), 4.40 (d, J=18.5 Hz, 1H), 4.32 (d, J=18.5 Hz, 1H), 2.37 (s, 3H).

### Synthesis Example 59—Synthesis of Compound 1059

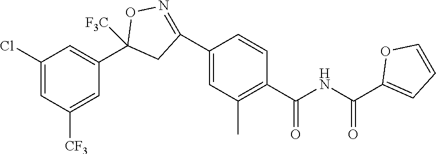 [SMILES: Cc1cc(C2=NOC(c3cc(Cl)cc(C(F)(F)F)c3)(C(F)(F)F)C2)ccc1C(=O)NC(=O)c1ccco1]

Compound 1059 was obtained through the same process as in Synthesis Example 1 except that 2-furancarbonyl chloride was used in place of cyclopropanecarbonyl chloride, and that SM2 was used in place of SM1.

¹H NMR (500 MHz, DMSO-d₆): δ 11.44 (s, 1H), 8.09 (t, J=1.9 Hz, 1H), 7.99 (dd, J=12.1, 2.0 Hz, 2H), 7.86 (s, 1H), 7.66 (d, J=1.8 Hz, 1H), 7.65-7.59 (m, 2H), 7.51 (d, J=8.1 Hz, TH), 6.72 (dd, J=3.6, 1.8 Hz, TH), 4.46 (d, J=18.3 Hz, TH), 4.38 (d, J=18.5 Hz, 1H), 2.37 (s, 3H).

### Synthesis Example 60—Synthesis of Compound 1060

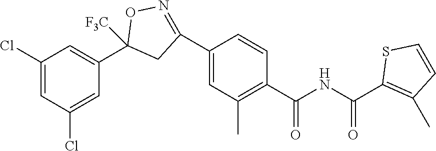 [SMILES: Cc1cc(C2=NOC(c3cc(Cl)cc(Cl)c3)(C(F)(F)F)C2)ccc1C(=O)NC(=O)c1sccc1C]

Compound 1060 was obtained through the same process as in Synthesis Example 1 except that 3-methyl-2-thiophenecarbonyl chloride was used in place of cyclopropanecarbonyl chloride.

¹H NMR (500 MHz, DMSO-d₆): δ 11.31 (s, 1H), 7.83-7.78 (m, 2H), 7.67-7.59 (m, 4H), 7.51 (d, J=8.1 Hz, 1H), 7.07-7.04 (m, 1H), 4.39 (d, J=18.3 Hz, 1H), 4.31 (d, J=18.5 Hz, 1H), 2.43 (s, 3H), 2.39 (s, 3H).

### Synthesis Example 61—Synthesis of Compound 1061

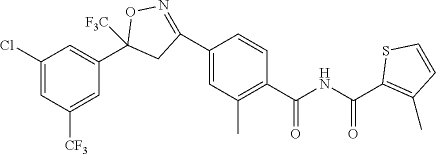 [SMILES: Cc1cc(C2=NOC(c3cc(Cl)cc(C(F)(F)F)c3)(C(F)(F)F)C2)ccc1C(=O)NC(=O)c1sccc1C]

Compound 1061 was obtained through the same process as in Synthesis Example 1 except that 3-methyl-2-thiophenecarbonyl chloride was used in place of cyclopropanecarbonyl chloride, and that SM2 was used in place of SM1.

¹H NMR (500 MHz, DMSO-d₆): δ 11.44-11.23 (m, 1H), 8.08 (t, J=2.0 Hz, 1H), 8.00-7.96 (m, 1H), 7.86 (s, 1H), 7.80 (dd, J=4.7, 2.3 Hz, 1H), 7.65 (d, J=1.8 Hz, 1H), 7.62 (dd, J=7.9, 2.0 Hz, 1H), 7.51 (dd, J=8.0, 2.4 Hz, 1H), 7.06 (d, J=5.0 Hz, 1H), 4.50-4.42 (m, 1H), 4.37 (d, J=18.5 Hz, 1H), 2.43 (s, 3H), 2.40 (s, 3H).

### Synthesis Example 62—Synthesis of Compound 1062

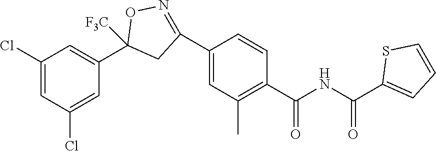 [SMILES: Cc1cc(C2=NOC(c3cc(Cl)cc(Cl)c3)(C(F)(F)F)C2)ccc1C(=O)NC(=O)c1cccs1]

Compound 1062 was obtained through the same process as in Synthesis Example 1 except that 2-thiophenecarbonyl chloride was used in place of cyclopropanecarbonyl chloride.

¹H NMR (500 MHz, DMSO-d₆): δ 11.62 (s, 1H), 8.16 (dd, J=3.9, 1.3 Hz, 1H), 7.99 (dd, J=5.0, 1.4 Hz, 1H), 7.81 (t, J=1.8 Hz, 1H), 7.67-7.58 (m, 4H), 7.51 (d, J=7.9 Hz, 1H), 7.23 (dd, J=5.0, 3.8 Hz, 1H), 4.40 (d, J=18.5 Hz, 1H), 4.32 (d, J=18.5 Hz, 1H), 2.37 (s, 3H).

### Synthesis Example 63—Synthesis of Compound 1063

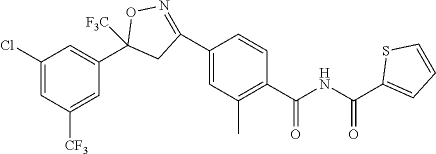 [SMILES: Cc1cc(C2=NOC(c3cc(Cl)cc(C(F)(F)F)c3)(C(F)(F)F)C2)ccc1C(=O)NC(=O)c1cccs1]

Compound 1063 was obtained through the same process as in Synthesis Example 1 except that 2-thiophenecarbonyl chloride was used in place of cyclopropanecarbonyl chloride, and that SM2 was used in place of SM1.

¹H NMR (500 MHz, DMSO-d₆): δ 11.62 (s, 1H), 8.16 (dt, J=3.2, 1.5 Hz, 1H), 8.09 (t, J=1.8 Hz, 1H), 7.99 (h, J=2.4, 1.9 Hz, 2H), 7.86 (s, 1H), 7.68-7.61 (m, 2H), 7.52 (d, J=7.9 Hz, 1H), 7.24 (dd, J=5.1, 3.7 Hz, 1H), 4.46 (d, J=18.3 Hz, 1H), 4.38 (d, J=18.5 Hz, 1H), 2.37 (s, 3H).

### Synthesis Example 64—Synthesis of Compound 1064

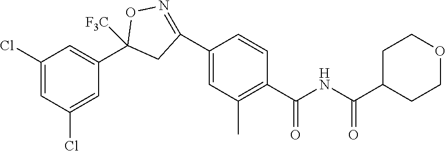 [SMILES: Cc1cc(C2=NOC(c3cc(Cl)cc(Cl)c3)(C(F)(F)F)C2)ccc1C(=O)NC(=O)C1CCOCC1]

Compound 1064 was obtained through the same process as in Synthesis Example 1 except that tetrahydro-2H-pyran-4-carbonyl chloride was used in place of cyclopropanecarbonyl chloride.

¹H NMR (500 MHz, DMSO-d₆): δ 11.10 (s, 1H), 7.81 (t, J=1.9 Hz, 1H), 7.63 (dd, J=5.4, 1.9 Hz, 4H), 7.47 (d, J=7.9 Hz, 1H), 4.42-4.36 (m, 1H), 4.31 (d, J=18.5 Hz, 1H), 3.87 (ddd, J=11.3, 4.3, 2.3 Hz, 2H), 3.35-3.33 (m, 1H), 3.29 (ddt, J=4.3, 2.7, 1.7 Hz, 1H), 2.89 (t, J=11.4 Hz, 1H), 2.35 (s, 3H), 1.73 (ddd, J=13.0, 4.0, 1.9 Hz, 2H), 1.56 (dtd, J=13.4, 11.7, 4.4 Hz, 2H).

### Synthesis Example 65—Synthesis of Compound 1065

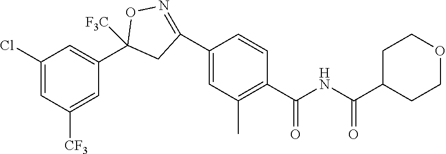 [SMILES: Cc1cc(C2=NOC(c3cc(Cl)cc(C(F)(F)F)c3)(C(F)(F)F)C2)ccc1C(=O)NC(=O)C1CCOCC1]

Compound 1065 was obtained through the same process as in Synthesis Example 1 except that tetrahydro-2H-pyran-4-carbonyl chloride was used in place of cyclopropanecarbonyl chloride, and that SM2 was used in place of SM1.

¹H NMR (500 MHz, DMSO-d₆): δ 11.11 (s, 1H), 8.09 (td, J=1.8, 0.8 Hz, 1H), 7.98 (t, J=1.9 Hz, 1H), 7.86 (s, 1H), 7.65-7.60 (m, 2H), 7.47 (d, J=7.9 Hz, 1H), 4.45 (d, J=18.3 Hz, 1H), 4.37 (d, J=18.6 Hz, 1H), 3.87 (ddd, J=11.3, 4.2, 2.3 Hz, 2H), 3.85-3.73 (m, 1H), 3.36-3.33 (m, 1H), 2.89 (tt, J=11.3, 3.7 Hz, 1H), 2.35 (s, 3H), 1.77-1.71 (m, 2H), 1.56 (dtd, J=13.4, 11.7, 4.3 Hz, 2H).

### Synthesis Example 66—Synthesis of Compound 1066

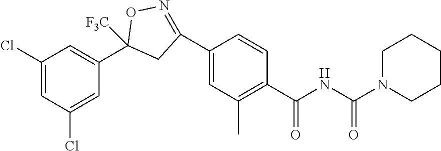 [SMILES: Cc1cc(C2=NOC(c3cc(Cl)cc(Cl)c3)(C(F)(F)F)C2)ccc1C(=O)NC(=O)N1CCCCC1]

Compound 1066 was obtained through the same process as in Synthesis Example 1 except that 1-piperidinecarbonyl chloride was used in place of cyclopropanecarbonyl chloride.

¹H NMR (500 MHz, DMSO-d₆): δ 10.28 (s, 1H), 7.81 (t, J=1.8 Hz, 1H), 7.64-7.57 (m, 4H), 7.43 (d, J=7.9 Hz, 1H), 4.38 (d, J=18.3 Hz, 1H), 4.30 (d, J=18.3 Hz, 1H), 3.43-3.34 (m, 4H), 2.37 (s, 3H), 1.62-1.44 (m, 6H).

### Synthesis Example 67—Synthesis of Compound 1067

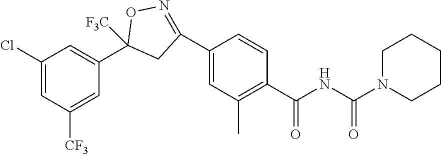 [SMILES: Cc1cc(C2=NOC(c3cc(Cl)cc(C(F)(F)F)c3)(C(F)(F)F)C2)ccc1C(=O)NC(=O)N1CCCCC1]

Compound 1067 was obtained through the same process as in Synthesis Example 1 except that 1-piperidinecarbonyl chloride was used in place of cyclopropanecarbonyl chloride, and that SM2 was used in place of SM1.

¹HNMR (500 MHz, DMSO-d₆): δ 10.29 (s, 1H), 8.09 (td, J=1.8, 0.7 Hz, 1H), 7.98 (t, J=1.8 Hz, 1H), 7.86 (s, 1H), 7.63-7.58 (m, 2H), 7.43 (d, J=7.9 Hz, 1H), 4.44 (d, J=18.5 Hz, 1H), 4.36 (d, J=18.5 Hz, 1H), 3.38 (t, J=5.6 Hz, 4H), 2.37 (s, 3H), 1.61-1.54 (m, 2H), 1.49 (d, J=3.7 Hz, 4H).

### Synthesis Example 68—Synthesis of Compound 1068

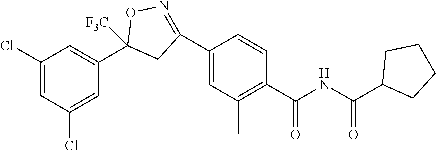 [SMILES: Cc1cc(C2=NOC(c3cc(Cl)cc(Cl)c3)(C(F)(F)F)C2)ccc1C(=O)NC(=O)C1CCCC1]

Compound 1068 was obtained through the same process as in Synthesis Example 1 except that cyclopentanecarbonyl chloride was used in place of cyclopropanecarbonyl chloride.

¹H NMR (500 MHz, DMSO-d₆): δ 11.06 (s, 1H), 7.82 (t, J=1.9 Hz, 1H), 7.65-7.59 (m, 4H), 7.45 (d, J=7.9 Hz, 1H), 4.42-4.27 (m, 2H), 3.12-3.03 (m, 1H), 2.34 (s, 3H), 1.88-1.80 (m, 2H), 1.68 (dtd, J=12.5, 5.2, 2.7 Hz, 2H), 1.63-1.58 (m, 2H), 1.53 (ddt, J=9.3, 4.4, 2.4 Hz, 2H).

### Synthesis Example 69—Synthesis of Compound 1069

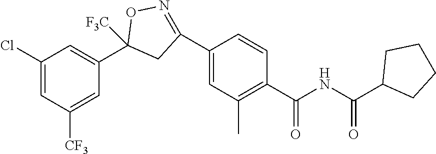 [SMILES: Cc1cc(C2=NOC(c3cc(Cl)cc(C(F)(F)F)c3)(C(F)(F)F)C2)ccc1C(=O)NC(=O)C1CCCC1]

Compound 1069 was obtained through the same process as in Synthesis Example 1 except that cyclopentanecarbonyl chloride was used in place of cyclopropanecarbonyl chloride, and that SM2 was used in place of SM1.

¹H NMR (500 MHz, DMSO-d₆): δ 12.01 (s, 1H), 8.09 (s, 1H), 7.98 (s, 1H), 7.86 (s, 1H), 7.61-7.57 (m, 2H), 7.46 (d, J=7.8 Hz, 1H), 4.48-4.32 (m, 2H), 2.65-2.60 (m, 1H), 2.40 (s, 3H), 1.80-1.76 (m, 2H), 1.67 (ddd, J=9.8, 6.1, 2.1 Hz, 2H), 1.59-1.56 (m, 2H), 1.53-1.49 (m, 2H).

### Synthesis Example 70—Synthesis of Compound 1070

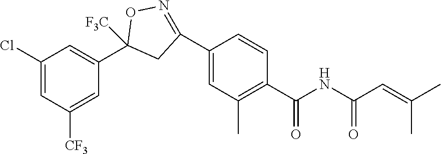 [SMILES: CC(C)=CC(=O)NC(=O)c1ccc(C2=NOC(c3cc(Cl)cc(C(F)(F)F)c3)(C(F)(F)F)C2)cc1C]

Compound 1070 was obtained through the same process as in Synthesis Example 1 except that 3-methyl-2-butenoyl chloride was used in place of cyclopropanecarbonyl chloride, and that SM2 was used in place of SM1.

¹H NMR (500 MHz, DMSO-d₆): δ 10.97 (s, 1H), 8.09 (s, 1H), 7.98 (s, 1H), is 7.86 (s, 1H), 7.64-7.62 (m, 1H), 7.60-7.57 (m, 1H), 7.45 (d, J=7.9 Hz, 1H), 6.18-6.14 (m, 1H), 4.45 (d, J=18.5 Hz, 1H), 4.40 (d, J=10.1 Hz, 1H), 2.34 (s, 3H), 2.08 (d, J=1.4 Hz, 3H), 1.89-1.87 (m, 3H).

### Synthesis Example 71—Synthesis of Compound 1071

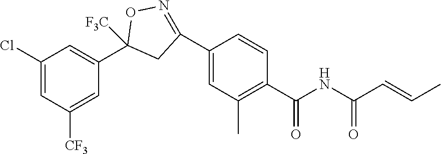 [SMILES: C/C=C/C(=O)NC(=O)c1ccc(C2=NOC(c3cc(Cl)cc(C(F)(F)F)c3)(C(F)(F)F)C2)cc1C]

Compound 1071 was obtained through the same process as in Synthesis Example 1 except that 2-butenoyl chloride was used in place of cyclopropanecarbonyl chloride, and that SM2 was used in place of SM1.

¹H NMR (500 MHz, DMSO-d₆): δ 11.12 (s, 1H), 8.09 (d, J=1.8 Hz, 1H), 7.98 (d, J=1.8 Hz, 1H), 7.86 (s, 1H), 7.68-7.59 (m, 2H), 7.49 (d, J=7.9 Hz, 1H), 6.92 (dq, J=15.3, 6.9 Hz, 1H), 6.44-6.38 (m, 1H), 4.49-4.42 (m, 1H), 4.38 (d, J=18.5 Hz, 1H), 2.41-2.33 (m, 3H), 1.87 (ddd, J=6.7, 5.0, 1.7 Hz, 3H).

### Synthesis Example 72—Synthesis of Compound 1072

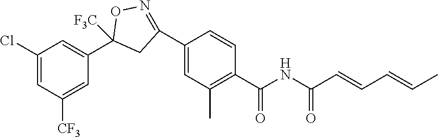 [SMILES: C/C=C/C=C/C(=O)NC(=O)c1ccc(C2=NOC(c3cc(Cl)cc(C(F)(F)F)c3)(C(F)(F)F)C2)cc1C]

Compound 1072 was obtained through the same process as in Synthesis Example 1 except that 2,4-hexadienoyl chloride was used in place of cyclopropanecarbonyl chloride, and that SM2 was used in place of SM1.

¹H NMR (500 Hz, CDCl₃): δ 8.18 (s, 1H), 7.80 (s, 1H), 7.74 (s, 1H), 7.67 (s, 1H), 7.58-7.53 (m, 2H), 7.50-7.43 (m, 2H), 6.87 (d, J=14.6 Hz, 1H), 6.36-6.21 (m, 2H), 4.12 (d, J=17.2 Hz, 1H), 3.71 (d, J=17.9 Hz, 1H), 2.49 (s, 3H), 1.89-1.86 (m, 3H).

### Synthesis Example 73—Synthesis of Compound 1073

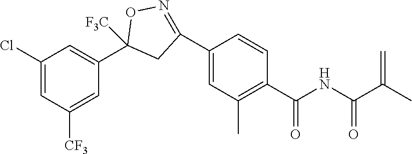 [SMILES: C=C(C)C(=O)NC(=O)c1ccc(C2=NOC(c3cc(Cl)cc(C(F)(F)F)c3)(C(F)(F)F)C2)cc1C]

Compound 1073 was obtained through the same process as in Synthesis Example 1 except that methacryloyl chloride was used in place of cyclopropanecarbonyl chloride, and that SM2 was used in place of SM1.

¹H NMR (500 MHz, DMSO-d₆): δ 11.14 (s, 1H), 8.09 (t, J=2.1 Hz, 1H), 7.98 (t, J=1.9 Hz, 1H), 7.86 (s, 1H), 7.65-7.62 (m, 1H), 7.60 (dd, J=7.9, 2.0 Hz, 1H), 7.46 (d, J=7.9 Hz, 1H), 5.99 (d, J=1.1 Hz, 1H), 5.71 (d, J=1.5 Hz, 1H), 4.45 (d, J=18.5 Hz, 1H), 4.37 (d, J=18.3 Hz, 1H), 2.34 (s, 3H), 1.85 (t, J=1.3 Hz, 3H).

### Synthesis Example 74—Synthesis of Compound 1074

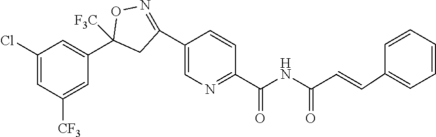 [SMILES: O=C(/C=C/c1ccccc1)NC(=O)c1ccc(C2=NOC(c3cc(Cl)cc(C(F)(F)F)c3)(C(F)(F)F)C2)cn1]

Compound 1074 was obtained through the same process as in Synthesis Example 1 except that cinnamoyl chloride was used in place of cyclopropanecarbonyl chloride, and that SM2 was used in place of SM1.

¹H NMR (500 Hz, CDCl₃): δ 8.22 (s, 1H), 7.89 (d, J=15.7 Hz, 1H), 7.81 (t, J=2.0 Hz, 1H), 7.74 (s, 1H), 7.70-7.66 (m, 1H), 7.64-7.52 (m, 6H), 7.40 (dd, J=5.1, 1.9 Hz, 3H), 4.13 (d, J=17.2 Hz, 1H), 3.72 (d, J=17.4 Hz, 1H), 2.53 (s, 3H).

### Synthesis Example 75—Synthesis of Compound 1075

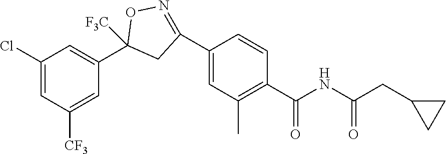 [SMILES: Cc1cc(C2=NOC(c3cc(Cl)cc(C(F)(F)F)c3)(C(F)(F)F)C2)ccc1C(=O)NC(=O)CC1CC1]

Compound 1075 was obtained through the same process as in Synthesis Example 1 except that cyclopropaneacetyl chloride was used in place of cyclopropanecarbonyl chloride, and that SM2 was used in place of SM1.

¹H NMR (500 Hz, CDCl₃): δ 8.26 (s, 1H), 7.80 (s, 1H), 7.73 (s, 1H), 7.67 (s, 1H), 7.58-7.53 (m, 2H), 7.47 (d, J=8.2 Hz, 1H), 4.12 (d, J=17.1 Hz, 1H), 3.74-3.68 (m, 1H), 2.78 (d, J=6.9 Hz, 2H), 2.48 (s, 3H), 1.16-1.07 (m, 1H), 0.64-0.59 (m, 2H), 0.25-0.20 (m, 2H).

### Synthesis Example 76—Synthesis of Compound 1076

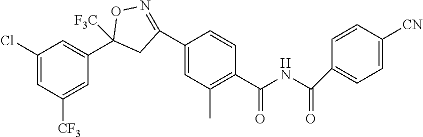 [SMILES: Cc1cc(C2=NOC(c3cc(Cl)cc(C(F)(F)F)c3)(C(F)(F)F)C2)ccc1C(=O)NC(=O)c1ccc(C#N)cc1]

Compound 1076 was obtained through the same process as in Synthesis Example 1 except that 4-cyanobenzoyl chloride was used in place of cyclopropanecarbonyl chloride, and that SM2 was used in place of SM1.

¹H NMR (500 Hz, CDCl₃): δ 8.83 (s, 1H), 7.96-7.91 (m, 2H), 7.80 (q, J=1.8 Hz, 2H), 7.78 (d, J=2.0 Hz, 1H), 7.74 (s, 1H), 7.68 (s, 1H), 7.60-7.56 (m, 2H), 7.48 (d, J=8.5 Hz, 1H), 4.13 (d, J=17.2 Hz, 1H), 3.71 (d, J=17.2 Hz, 1H), 2.47 (s, 3H).

### Synthesis Example 77—Synthesis of Compound 1077

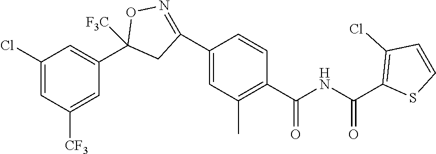 [SMILES: Cc1cc(C2=NOC(c3cc(Cl)cc(C(F)(F)F)c3)(C(F)(F)F)C2)ccc1C(=O)NC(=O)c1sccc1Cl]

Compound 1077 was obtained through the same process as in Synthesis Example 1 except that 3-chloro-2-thiophenecarbonyl chloride was used in place of cyclopropanecarbonyl chloride, and that SM2 was used in place of SM1.

¹H NMR (500 Hz, CDCl₃): δ 9.58 (s, 1H), 7.80 (s, 1H), 7.74 (s, 1H), 7.67 (s, is 1H), 7.63 (d, J=5.3 Hz, 1H), 7.59-7.56 (m, 2H), 7.51-7.47 (m, 1H), 7.05 (d, J=5.3 Hz, 1H), 4.13 (d, J=17.2 Hz, 1H), 3.71 (d, J=17.2 Hz, 1H), 2.49 (s, 3H).

### Synthesis Example 78—Synthesis of Compound 1078

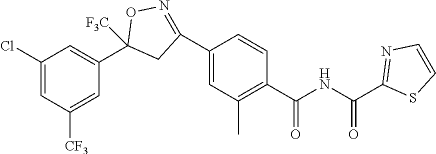 [SMILES: Cc1cc(C2=NOC(c3cc(Cl)cc(C(F)(F)F)c3)(C(F)(F)F)C2)ccc1C(=O)NC(=O)c1nccs1]

Compound 1078 was obtained through the same process as in Synthesis Example 1 except that 2-thiazolecarbonyl chloride was used in place of cyclopropanecarbonyl chloride, and that SM2 was used in place of SM1.

¹H NMR (500 Hz, CDCl₃): δ 10.03 (s, 1H), 7.94 (d, J=2.9 Hz, 1H), 7.81 (t, J=1.9 Hz, 1H), 7.75 (s, 1H), 7.74 (d, J=2.9 Hz, 1H), 7.69-7.67 (m, 1H), 7.59 (dd, J=7.4, 0.7 Hz, 2H), 7.57-7.51 (m, 1H), 4.14 (d, J=17.1 Hz, 1H), 3.72 (d, J=17.2 Hz, 1H), 2.52 (s, 3H).

### Synthesis Example 79—Synthesis of Compound 1079

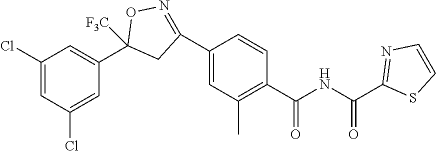 [SMILES: Cc1cc(C2=NOC(c3cc(Cl)cc(Cl)c3)(C(F)(F)F)C2)ccc1C(=O)NC(=O)c1nccs1]

Compound 1079 was obtained through the same process as in Synthesis Example 1 except that 2-thiazolecarbonyl chloride was used in place of cyclopropanecarbonyl chloride.

¹H NMR (500 Hz, CDCl₃): δ 10.04 (s, 1H), 7.94 (d, J=2.9 Hz, 1H), 7.73 (d, J=2.9 Hz, 1H), 7.58 (dd, J=7.3, 0.8 Hz, 2H), 7.56-7.52 (m, 1H), 7.50 (d, J=2.0 Hz, 2H), 7.42 (t, J=1.8 Hz, 1H), 4.08 (d, J=17.2 Hz, 1H), 3.69 (d, J=17.2 Hz, 1H), 2.51 (s, 3H).

### Synthesis Example 80—Synthesis of Compound 1080

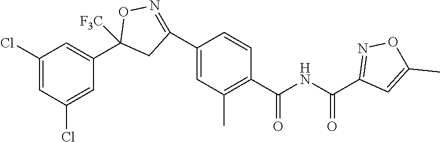 [SMILES: Cc1cc(C(=O)NC(=O)c2ccc(C3=NOC(c4cc(Cl)cc(Cl)c4)(C(F)(F)F)C3)cc2C)no1]

Compound 1080 was obtained through the same process as in Synthesis Example 1 except that 5-methyl-3-isoxazolecarbonyl chloride was used in place of cyclopropanecarbonyl chloride.

¹H NMR (500 MHz, DMSO-d₆): δ 11.43 (s, 1H), 9.07 (d, J=0.9 Hz, 1H), 7.82 (t, J=1.9 Hz, 1H), 7.66 (s, 1H), 7.62 (dd, J=8.2, 2.0 Hz, 3kiikiH), 7.55 (d, J=8.1 Hz, 1H), 4.40 (d, J=18.5 Hz, 1H), 4.32 (d, J=18.5 Hz, 1H), 2.61 (s, 3H), 2.37 (s, 3H).

### Synthesis Example 81—Synthesis of Compound 1081

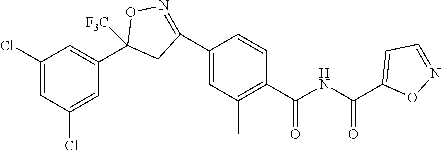 [SMILES: Cc1cc(C2=NOC(c3cc(Cl)cc(Cl)c3)(C(F)(F)F)C2)ccc1C(=O)NC(=O)c1ccno1]

Compound 1081 was obtained through the same process as in Synthesis Example 1 except that 5-isoxazolecarbonyl chloride was used in place of cyclopropanecarbonyl chloride.

¹H NMR (500 Hz, CDCl₃): δ 9.09 (s, 1H), 8.41 (d, J=1.8 Hz, 1H), 7.58 (dt,J=7.6, 1.3 Hz, 2H), 7.53-7.50 (m, 1H), 7.50 (d, J=2.0 Hz, 2H), 7.42 (q, J=2.0 Hz, 1H), 7.09 (d, J=1.8 Hz, 1H), 4.08 (d, J=17.1 Hz, 1H), 3.69 (d, J=17.2 Hz, 1H), 2.51 (s, 3H).

### Synthesis Example 82—Synthesis of Compound 1082

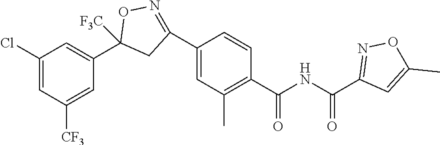 [SMILES: Cc1cc(C(=O)NC(=O)c2ccc(C3=NOC(c4cc(Cl)cc(C(F)(F)F)c4)(C(F)(F)F)C3)cc2C)no1]

Compound 1082 was obtained through the same process as in Synthesis Example 1 except that 5-methyl-3-isoxazolecarbonyl chloride was used in place of cyclopropanecarbonyl chloride, and that SM2 was used in place of SM1.

¹H NMR (500 MHz, DMSO-d₆): δ 8.67 (s, 1H), 8.55 (d, J=0.8 Hz, 1H), 7.80 (s, 1H), 7.74 (s, 1H), 7.68 (s, 1H), 7.58 (ddd, J=3.8, 2.4, 0.6 Hz, 2H), 7.49-7.46 (m, 1H), 4.13 (d, J=17.1 Hz, 1H), 3.71 (d, J=17.2 Hz, 1H), 2.68 (d, J=0.8 Hz, 3H), 2.47 (s, 3H).

### Synthesis Example 83—Synthesis of Compound 1083

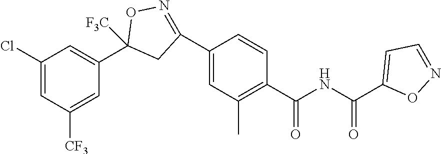 [SMILES: Cc1cc(C2=NOC(c3cc(Cl)cc(C(F)(F)F)c3)(C(F)(F)F)C2)ccc1C(=O)NC(=O)c1ccno1]

Compound 1083 was obtained through the same process as in Synthesis Example 1 except that 5-isoxazolecarbonyl chloride was used in place of cyclopropanecarbonyl chloride, and that SM2 was used in place of SM1.

¹H NMR (500 Hz, CDCl₃): δ 9.08 (s, 1H), 8.41 (d, J=1.8 Hz, 1H), 7.81 (s, 1H), 7.74 (s, 1H), 7.68 (s, 1H), 7.59 (ddd, J=7.2, 1.7, 0.8 Hz, 2H), 7.53-7.49 (m, 1H), 7.09 (d, J=1.8 Hz, 1H), 4.14 (d, J=17.1 Hz, 1H), 3.72 (d, J=17.2 Hz, 1H), 2.51 (s, 3H).

### Synthesis Example 84—Synthesis of Compound 1084

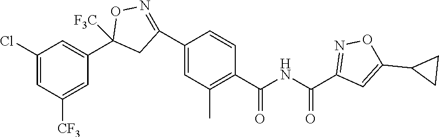 [SMILES: Cc1cc(C2=NOC(c3cc(Cl)cc(C(F)(F)F)c3)(C(F)(F)F)C2)ccc1C(=O)NC(=O)c1cc(C2CC2)on1]

Compound 1084 was obtained through the same process as in Synthesis Example 1 except that 5-cyclopropyl-3-isoxazolecarbonyl chloride was used in place of cyclopropanecarbonyl chloride, and that SM2 was used in place of SM1.

¹H NMR (500 Hz, CDCl₃): δ 9.39 (s, 1H), 7.80 (s, 1H), 7.74 (s, 1H), 7.68 (s, 1H), 7.57 (ddd, J=6.7, 1.7, 0.7 Hz, 2H), 7.50 (d, J=8.7 Hz, 1H), 6.37 (s, 1H), 4.13 (d, J=17.1 Hz, 1H), 3.71 (d, J=17.2 Hz, 1H), 2.50 (s, 3H), 2.08 (tt, J=8.5, 5.0 Hz, 1H), 1.17-1.12 (m, 2H), 1.03-0.97 (m, 2H).

### Synthesis Example 85—Synthesis of Compound 1085

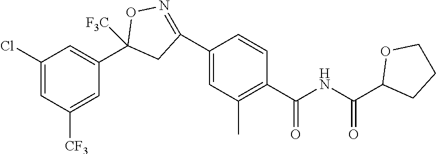 [SMILES: Cc1cc(C2=NOC(c3cc(Cl)cc(C(F)(F)F)c3)(C(F)(F)F)C2)ccc1C(=O)NC(=O)C1CCCO1]

Compound 1085 was obtained through the same process as in Synthesis Example 1 except that tetrahydro-2-furancarbonyl chloride was used in place of cyclopropanecarbonyl chloride, and that SM2 was used in place of SM1.

¹H NMR (500 Hz, CDCl₃): δ 9.35 (s, 1H), 7.80 (s, 1H), 7.73 (s, 1H), 7.67 (s, 1H), 7.57-7.53 (m, 2H), 7.41 (d, J=8.5 Hz, 1H), 4.44 (dd, J=8.5, 6.0 Hz, 1H), 4.12 (d, J=17.5 Hz, 1H), 4.00 (ddd, J=8.5, 7.1, 6.0 Hz, 1H), 3.92 (dt, J=8.5, 6.9 Hz, 1H), 3.70 (d, J=17.2 Hz, 1H), 2.46 (s, 3H), 2.38-2.29 (m, 1H), 2.17-2.06 (m, 1H), 2.02-1.85 (m, 2H).

### Examples 1 to 85—Preparation of Pesticide Compositions

Pesticide compositions were prepared by adjusting the concentration of each compound synthesized in Synthesis Examples 1 to 85 (e.g., 100 ppm, 10 ppm, 3 ppm, 1 ppm, 0.3 ppm, and 0.1 ppm). In this case, distilled water and/or acetone were used as the solvent, and Triton X-100 was used as the surfactant.

### Comparative Example 1—Preparation of Pesticide Composition

A pesticide composition was prepared by adjusting the concentration of a compound (fluxametamide) represented by Formula below. In particular, distilled water and/or acetone were used as the solvent, and Triton X-100 was used as the surfactant.

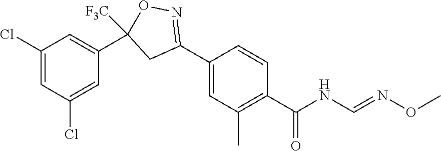 [SMILES: CO/N=C/NC(=O)c1ccc(C2=NOC(c3cc(Cl)cc(Cl)c3)(C(F)(F)F)C2)cc1C]

### Comparative Example 2—Preparation of Pesticide Composition

A pesticide composition was prepared by adjusting the concentration of a compound (chlorantraniliprole) represented by Formula below. In this case, distilled water and/or acetone were used as the solvent, and Triton X-100 was used as the surfactant.

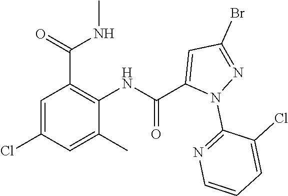 [SMILES: CNC(=O)c1cc(Cl)cc(C)c1NC(=O)c1cc(Br)nn1-c1ncccc1Cl]

### Comparative Example 3—Preparation of Pesticide Composition

A pesticide composition was prepared by adjusting the concentration of a compound (isocycloseram) represented by Formula below. In particular, distilled water and/or acetone were used as the solvent, and Triton X-100 was used as the surfactant.

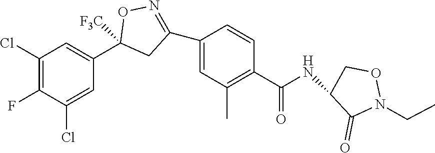 [SMILES: CCN1OC[C@@H](NC(=O)c2ccc(C3=NO[C@@](c4cc(Cl)c(F)c(Cl)c4)(C(F)(F)F)C3)cc2C)C1=O]

### Test Example 1—Test of Pesticidal Activity Against Plutella xylostella Through Leaf-Dipping Method

Cabbage (Daiya) leaves were cut into pieces with a diameter of 5.8 cm, dipped in each of the pesticide compositions prepared in Examples (a 5% acetone solution in which each of the compounds of Synthesis Examples (concentration: 100 ppm) was diluted) for 30 seconds, and sufficiently shade-dried. Then, the shade-dried cabbage leaves were placed in a petri dish (8.8 cm in diameter) covered with a filter paper and repeatedly inoculated 3 times with third instar larvae of Plutella xylostella, 10 larvae per inoculation. In particular, Plutella xylostella larvae used were those which were collected in a place near Gyeongju (Korea) in 2000 and bred in a breeding room for multiple generations. Then, the Plutella xylostella larvae were stored under the conditions of 16 hours of light and 8 hours of dark, 25±1° C., and relative humidity of 50-60%, and the number of live Plutella xylostella larvae was counted 48 hours after the inoculation. Thereafter, the larvae mortality rate was calculated by correcting the density after treatment based on the density before treatment, as shown in Equation 1 and Equation 2 below, and converting the same into the corrected mortality rate for untreated (Reference: A method of computing the effectiveness of an insecticide. J. Econ. Entomol. 18:265˜267. Abbott, 1925).

$$mortality rate of larvae ( control value , \% ) = \{ ( survival rate of larvae by non - treatment - survival rate of larvae by treated group ) / ( survival rate of larvae by non - treatment ) \} \times 100 \qquad [ Equation 1 ]$$

$$survival rate of larvae = ( density of larvae after treatment / density of larvae before treatment ) \times 100 \qquad [ Equation 2 ]$$

As a result of performing tests using each of the compounds of Synthesis Examples 1 to 85, the pesticide compositions prepared respectively using the compounds of Synthesis Examples 1, 1a, 1b, 2, 2a, 2b, 3, 3a, 3b, 4, 5, 6, 8, 9, 10, 11, 12, 13, 14, 17, 18, 19, 20, 21, 22, 23, 24, 25, 26, 27, 28, 29, 30, 31, 32, 33, 37, 39, 40, 45, 46, 47, 49, 50, 52, 53, 55, 56, 57, 58, 59, 65, 66, 67, 68, 69, 70, 71, 72, 75, 76, 78, 79, 80, 81, 82, 83, 84, and 85 showed a larvae mortality rate of 80% or more against Plutella xylostella.

Meanwhile, the pesticide composition according to Example 2 prepared using the compounds of Synthesis Example 2 and the pesticide compositions according to Comparative Examples 1 and 2 were tested for pesticidal activity against Plutella xylostella in the same manner, and the results are shown in Table 1 below.

### Test Example 2—Pesticidal Activity Against Spodoptera litura Through Leaf-Dipping Method

Cabbage (Daiya) leaves were cut into pieces with a diameter of 5.8 cm, dipped in each of the pesticide compositions prepared in Examples (a 5% acetone solution in which each of the compounds of Synthesis Examples (concentration: 100 ppm) was diluted) for 30 seconds, and sufficiently shade-dried. Then, the shade-dried cabbage leaves were placed in a petri dish (8.8 cm in diameter) covered with a filter paper and repeatedly inoculated 3 times with the second instar larvae of Spodoptera litura, 10 larvae per inoculation. In this case, Spodoptera litura larvae used were those which were purchased from the Bio Utilization Institute (Andong, Korea). Then, the Spodoptera litura larvae were stored under the conditions of 16 hours of light and 8 hours of dark, 25±1° C., and relative humidity of 50-60%, and the number of live Spodoptera litura larvae was counted 48 hours after the inoculation. Thereafter, the larvae mortality rate was calculated in the same manner as in Test Example 1 above.

As a result of performing tests using each of the compounds of Synthesis Examples 1 to 85, the pesticide compositions prepared respectively using the compounds of Synthesis Examples 1, 1a, 1b, 2, 2a, 2b, 3, 3a, 3b, 4, 5, 6, 8, 9, 10, 11, 12, 13, 14, 17, 18, 19, 20, 21, 22, 23, 24, 25, 26, 27, 28, 29, 30, 31, 32, 33, 37, 39, 40, 45, 46, 47, 49, 50, 52, 53, 55, 56, 57, 58, 59, 65, 66, 67, 68, 69, 70, 71, 72, 75, 76, 78, 79, 80, 81, 82, 83, 84, and 85 showed a mortality rate of 80% or more against Spodoptera litura.

Meanwhile, the pesticide compositions according to Example 2 prepared using the compounds of Synthesis Example 2 and the pesticide compositions according to Comparative Examples 1 and 2 were tested for pesticidal activity against Spodoptera litura in the same manner, and the results are shown in Table 1 below.

### Test Example 3—Test of Pesticidal Activity Against Frankliniella occidentalis Through Spray Method

A water-soaked filter paper (9.0 cm in diameter) was placed in an insect breeding dish (Lab Guide®) with a diameter of 9.0 cm and a height of 4.0 cm, and then parafilm (width 4.0 cm×height 4.0 cm) was placed thereon. Before treating with a pesticide composition, 10 adult Frankliniella occidentalis were inoculated per petri dish using a No. 4 brush. In this case, Frankliniella occidentalis adults used were those purchased from the Bio Utilization Institute (Andong, Korea). Then, each of the pesticide compositions prepared in Examples (a diluted solution in which each of the compounds of Synthesis Examples (concentration: 100 ppm) was diluted; the solvent=distilled water+acetone 50,000 mg/L+Triton X-100 100 mg/L) was loaded into a 100 mL small sprayer, and sprayed 10-12 times at a distance of 30 cm and a height of 50 cm, and one cotyledons of soybean, which is a host plant, were added one at a time. Then, the Frankliniella occidentalis adults were stored under the conditions of 16 hours of light and 8 hours of darkness, 25±1° C., and relative humidity of 50-60%, and the number of live Frankliniella occidentalis adults was counted 48 hours after the inoculation. Thereafter, the larvae mortality rate was calculated in the same manner as in Test Example 1 above.

As a result of performing tests using each of the compounds of Synthesis Examples 1 to 85, the pesticide compositions prepared respectively using the compounds of Synthesis Examples 1, 1a, 1b, 2, 2a, 2b, 3, 3a, 3b, 4, 5, 6, 8, 9, 10, 11, 12, 13, 14, 17, 18, 19, 20, 21, 22, 23, 24, 25, 26, 27, 28, 29, 30, 31, 32, 33, 37, 39, 40, 45, 46, 47, 49, 50, 52, 53, 55, 56, 57, 58, 59, 65, 66, 67, 68, 69, 70, 71, 72, 75, 76, 78, 79, 80, 81, 82, 83, 84, and 85 showed a mortality rate of 80% or more against Frankliniella occidentalis.

Meanwhile, the pesticide compositions according to Example 2 prepared using the compounds of Synthesis Example 2 and the pesticide compositions according to Comparative Examples 1 and 2 were tested for pesticidal activity against Frankliniella occidentalis in the same manner, and the results are shown in Table 1 below.

### Test Example 4—Test of Pesticidal Activity Against Spodoptera exigua Through Leaf-Dipping Method

Cabbage (Daiya) leaves were cut into pieces with a diameter of 5.8 cm, dipped in the pesticide composition where the compound of Synthesis Example 2 was diluted (the solvent=distilled water+acetone 50,000 mg/L+Triton X-100 100 mg/L) for 30 seconds, and sufficiently shade-dried. Then, the shade-dried cabbage leaves were placed in a petri dish (8.8 cm in diameter) covered with filter paper and repeatedly inoculated 3-5 times with the second to the third instar larvae of Spodoptera exigua, 10 larvae per inoculation. In particular, the Spodoptera exigua larvae used were those purchased from the Bio Utilization Institute (Andong, Korea). Then, the Spodoptera exigua larvae were stored under the conditions of 16 hours of light and 8 hours of dark, 25±1° C. and 50-60% relative humidity, and the number of live Spodoptera exigua larvae was counted 24, 48, and 72 hours after the inoculation. Thereafter, the larvae mortality rate was calculated in the same manner as in Test Example 1 above.

Meanwhile, tests were performed using the pesticide compositions according to Comparative Examples 1 and 2 under the same conditions, and the results are shown in Table 1 below.

### Test Example 5—Test of Pesticidal Activity Against Spodoptera frugiperda Through Leaf-Dipping Method

Cabbage (Daiya) leaves were cut into pieces with a diameter of 5.8 cm, dipped in the pesticide composition where the compound of Synthesis Example 2 was diluted (the solvent=distilled water+acetone 50,000 mg/L+Triton X-100 100 mg/L) for 30 seconds, and sufficiently shade-dried. Then, the shade-dried cabbage leaves were placed in a petri dish (8.8 cm in diameter) covered with filter paper and repeatedly inoculated 3-5 times with the second to the third instar larvae of Spodoptera frugiperda, 10 larvae per inoculation. In particular, the Spodoptera frugiperda larvae used were those which were provided by Chung Buk University (Korea) and bred indoors for multiple generations. Then, the Spodoptera frugiperda larvae were stored under the conditions of 16 hours of light and 8 hours of dark, 25±1° C. and 50-60% relative humidity, and the number of live Spodoptera frugiperda larvae was counted 24, 48, and 72 hours after the inoculation. Thereafter, the larvae mortality rate was calculated in the same manner as in Test Example 1 above.

Meanwhile, a test was performed using the pesticide composition according to Comparative Example 1 under the same conditions, and the results are shown in Table 1 below.

### Test Example 6—Test of Pesticidal Activity Against Maruca vitrta Through Artificial Feed-Dipping Method

Cabbage (Daiya) leaves were cut into pieces with a size of 1 cm×1 cm×1 cm, dipped in the pesticide composition where the compound of Synthesis Example 2 was diluted (the solvent=distilled water+acetone 50,000 mg/L+Triton X-100 100 mg/L) for 30 seconds, and then sufficiently shade-dried by placing them on aluminum foil. In this case, the artificial feed used was prepared by a process, in which (1) 13 g of agar powder and 625 mL of secondary distilled water were added into a 1 L beaker and boiled in the microwave for 10 minutes; (2) the agar boiled in the microwave was added into a blender and then 75 g of soybean powder, 20 g of red bean powder, 10 g of malt powder, and 175 mL of secondary distilled water were added thereto and mixed together; and (3) after the resulting mixture was cooled to 50° C., 5 g of a vitamin mixture, 4 g of ascorbic acid, 1 g of sorbic acid, 1 g of β-sitosterol, 10 g of glucose, 10 g of cellulose, 3 g of cholesterol, 1.5 g of methyl-p-hydroxybenzoate, 0.5 g of aureomycin, and 0.4 g of fumidil B were added thereto, mixed together, and then thoroughly cooled. Before placing the artificial feed pieces in a petri dish (¢90 mm×15 mm), parafilm (5.1 cm×5.1 cm) was placed on top of a distilled water-treated filter paper (Ø90 mm) to maintain humidity, and the artificial feed pieces were placed on top of the parafilm.

Then, the second to the third instar larvae of Maruca vitrta were repeatedly inoculated 3-5 times, 10 larvae per inoculation. In this case, the Maruca vitrta larvae used were those purchased from the Bio Utilization Institute (Andong, Korea). Then, the Maruca vitrta larvae were stored under 16 hours of light: 8 hours of dark, 25±1° C., and 50-60% relative humidity, and the number of live Maruca vitrta larvae was counted 24, 48, and 72 hours after inoculation. Thereafter, the larvae mortality rate was calculated in the same manner as in Test Example 1 above.

Meanwhile, tests were performed using the pesticide compositions according to Comparative Examples 1 and 2 under the same conditions, and the results are shown in Table 1 below.

### Test Example 7—Test of Pesticidal Activity Against Phaedon brassicae Through Leaf-Dipping Method

Cabbage (Daiya) leaves were cut into pieces with a diameter of 5.8 cm, dipped in the pesticide composition where the compound of Synthesis Example 2 was diluted (the solvent=distilled water+acetone 50,000 mg/L+Triton X-100 100 mg/L) for 30 seconds, and sufficiently shade-dried. Then, the shade-dried cabbage leaves were is placed in a petri dish (8.8 cm in diameter) covered with filter paper and repeatedly inoculated 3-5 times with the larvae of Phaedon brassicae, 10 larvae per inoculation. In particular, the Phaedon brassicae larvae used were those which were bred in the Andong National University (Korea) for multiple generations.

After the inoculation, the Phaedon brassicae larvae were stored under the conditions of 16 hours of light and 8 hours of dark, 25±1° C. and 50-60% relative humidity, and the number of live Phaedon brassicae larvae was counted 24, 48, and 72 hours after the inoculation. Thereafter, the larvae mortality rate was calculated in the same manner as in Test Example 1 above.

Meanwhile, tests were performed using the pesticide compositions according to Comparative Examples 1 and 2 under the same conditions, and the results are shown in Table 1 below.

### Test Example 8—Test of Pesticidal Activity Against Thrips tabaci Lindeman Through Spray Method

A water-soaked filter paper (9.0 cm in diameter) was placed in an insect breeding dish (Lab Guide®) with a diameter of 9.0 cm and a height of 4.0 cm, and then parafilm (width 4.0 cm×height 4.0 cm) was placed thereon. Before being treated with a pesticide composition, 10 adult Thrips tabaci Lindeman were inoculated per petri dish using a No. 4 brush. In this case, Thrips tabaci Lindeman adults used were those collected in a place near the Andong National University (Korea) and bred for multiple generations. Then, the pesticide composition where the compound of Synthesis Example 2 was diluted (the solvent=distilled water+acetone 50,000 mg/L+Triton X-100 100 mg/L) was loaded into a 100 mL small sprayer, and sprayed 10-12 times at a distance of 30 cm and a height of 50 cm, and one cotyledons of soybean, which is a host plant, were added one at a time. Then, the Thrips tabaci Lindeman adults were stored under the conditions of 16 hours of light and 8 hours of darkness, 25±1° C., and relative humidity of 50-60%, and the number of live Thrips tabaci Lindeman adults was counted 24, and 48 hours after the inoculation. Thereafter, the larvae mortality rate was calculated in the same manner as in Test Example 1 above.

Meanwhile, tests were performed using the pesticide compositions according to Comparative Examples 1 and 3 under the same conditions, and the results are shown in Table 1 below.

### Test Example 9—Test of Pesticidal Activity Against Lymantria dispar Through Leaf-Dipping Method

Maple leaves were dipped in the pesticide composition where the compound of Synthesis Example 2 was diluted (the solvent=distilled water+acetone 50,000 mg/L+Triton X-100 100 mg/L) for 30 seconds, and sufficiently shade-dried by placing them on aluminum foil. Then, in order to maintain the humidity of the shade-dried maple leaves, they were placed on a petri dish (9.0 cm in diameter) covered with a filter paper (9.0 cm in diameter) treated with distilled water, and repeatedly inoculated 4 times of five early third instar larvae of Lymantria dispar. In particular, Lymantria dispar larvae used were collected in Jukjang-myeon, Buk-gu, Pohang-si, Gyeongsangbuk-do, Korea. Then, Lymantria dispar larvae were stored under 16 hours of light: 8 hours of dark, 25±1° C., and 50-60% relative humidity, and the number of Lymantria dispar larvae was counted 24, 48, and 72 hours after inoculation. Thereafter, the larvae mortality rate was calculated in the same manner as in Test Example 1 above.

Meanwhile, tests were performed using the pesticide compositions according to Comparative Examples 1 and 3 under the same conditions, and the results are shown in Table 1 below.

### Test Example 10—Test of Pesticidal Activity Against Grapholita molesta Through Spray Method

The pesticide composition where the compound of Synthesis Example 2 was diluted (the solvent=distilled water+acetone 50,000 mg/L+Triton X-100 100 mg/L) was directly sprayed on apples using a CO₂ Sprayer® [a nozzle type: Teejet8002VS (a flat fan type), pressure: 800 psi), and then apples were collected 1 hour later, and Grapholita molesta larvae were repeatedly inoculated 5-6 times, 3 per apple. In particular, the Grapholita molesta larvae were collected in the R&D Center of Kyung Nong Co., Ltd. (Gyeongju, Korea), and they were used after acclimatization indoors for 24 hours. Then, the Grapholita molesta larvae were stored under the conditions of 16 hours of light and 8 hours of dark, 25±1° C. and relative humidity of 50-60%, and the number of live Grapholita molesta larvae was counted 24, 72, and 192 hours after the inoculation. Thereafter, the larvae mortality rate was calculated in the same manner as in Test Example 1 above.

Meanwhile, a test was performed using the pesticide composition according to Comparative Example 1 under the same conditions, and the results are shown in Table 1 below.

### Test Example 11—Test of Pesticidal Activity Against Rhopobota Naevana Through Spray Method

Apple leaves were collected along with the stems, and the leaves of 3-4 base were cut into 5 cm×5 cm×5 cm pieces and placed in a tray oasis submerged in water. Then, the pesticide composition where the compound of Synthesis Example 2 was diluted (the solvent=distilled water+acetone 50,000 mg/L+Triton X-100 100 mg/L) was loaded into a 150 mL small sprayer and sprayed to the apple leaves, and 1 hour thereafter, the Grapholita molesta larvae were repeatedly inoculated 5-6 times, one per apple leaf. In particular, the Rhopobota naevana larvae were collected in the R&D Center of Kyung Nong Co., Ltd. (Gyeongju, Korea), and they were used after acclimatization indoors for 24 hours. Then, the Rhopobota naevana larvae were stored under the conditions of 16 hours of light and 8 hours of dark, 25±1° C. and relative humidity of 50-60%, and the number of live Rhopobota naevana larvae was counted 24, 48, and 72 hours after the inoculation. Thereafter, the larvae mortality rate was calculated in the same manner as in Test Example 1 above.

Meanwhile, a test was performed using the pesticide composition according to Comparative Example 1 under the same conditions, and the results are shown in Table 1 below.

### Test Example 12—Test of Pesticidal Activity Against Manulea Degenerella Through Spray Method

A water-soaked filter paper (9.0 cm in diameter) was placed in an insect breeding dish (Lab Guide®) with a diameter of 9.0 cm and a height of 4.0 cm, and then parafilm (width 4.0 cm×height 4.0 cm) was placed thereon. Before treating with a pesticide composition, 10 of the third instar Manulea degenerella larvae were inoculated per breeding dish using a No. 4 brush. In particular, Manulea degenerella larvae used were those collected in Icheon, Gyeonggi-do (Korea). Then, the pesticide composition where the compound of Synthesis Example 2 was diluted (the solvent=distilled water+acetone 50,000 mg/L+Triton X-100 100 mg/L) was loaded into a 100 mL small sprayer, and sprayed 10-12 times at a distance of 30 cm and a height of 50 cm, and one cotyledons of a persimmon tree, which is a host plant, were added one at a time. Then, the Manulea degenerella larvae were stored under the conditions of 16 hours of light and 8 hours of darkness, 25±1° C., and relative humidity of 50-60%, and the number of live Manulea degenerella larvae was counted 24, 48, and 72 hours after the inoculation. Thereafter, the larvae mortality rate was calculated in the same manner as in Test Example 1 above.

Meanwhile, a test was performed using the pesticide composition according to Comparative Example 1 under the same conditions, and the results are shown in Table 1 below.

### Test Example 13—Test of Pesticidal Activity Against Helicoverpa armigera Through Leaf-Dipping Method

Cabbage (Daiya) leaves were cut into pieces with a diameter of 5.8 cm, dipped in the pesticide composition where the compound of Synthesis Example 2 was diluted (the solvent=distilled water+acetone 50,000 mg/L+Triton X-100 100 mg/L) for 30 seconds, and sufficiently shade-dried. Then, the shade-dried cabbage leaves were placed in a petri dish (8.8 cm in diameter) covered with a filter paper and repeatedly inoculated 3-5 times with third instar larvae of Helicoverpa armigera, 10 larvae per inoculation. In this case, Helicoverpa armigera larvae used were those purchased from the Andong National University (Korea). Then, the Helicoverpa armigera larvae were stored under the conditions of 16 hours of light and 8 hours of dark, 25±1° C., and relative humidity of 50-60%, and the number of live Helicoverpa armigera larvae was counted 24, 48, and 72 hours after the inoculation. Thereafter, the larvae mortality rate was calculated in the same manner as in Test Example 1 above.

Meanwhile, a test was performed using the pesticide composition according to Comparative Example 1 under the same conditions, and the results are shown in Table 1 below.

TABLE 1

| | Concentration of | | | | | |
|---|---|---|---|---|---|---|
| | Compound in | | Example 2 | Comparative | Comparative | Comparative |
| | Pesticide | | (Synthesis | Example 1 | Example 2 | Example 3 |
| Pests | Composition | | Example 2) | (fluxametamide) | (chlorantraniliprole) | (isocycloseram) |
| Plutella | 10 | ppm | 100% | 100% | 100% | — |
| xylostella | 1 | ppm | 100% | 100% | 100% | — |
| | 0.1 | ppm | 100% | 100% | 21% | — |
| Spodoptera | 10 | ppm | 100% | 100% | — | — |
| litura | 1 | ppm | 100% | 100% | — | — |
| | 0.1 | ppm | 100% | 90% | — | — |
| Frankliniella | 10 | ppm | 100% | 50% | — | 100% |
| occidentalis | 5 | ppm | 100% | 13% | — | 100% |
| | 1 | ppm | 100% | 7% | — | — |
| Spodoptera | 10 | ppm | 100% | 100% | 71% | — |
| exigua | 1 | ppm | 100% | 100% | 46% | — |
| Spodoptera | 10 | ppm | 100% | 100% | — | — |
| frugiperda | 1 | ppm | 100% | 100% | — | — |
| | 0.1 | ppm | 100% | 100% | — | — |
| Maruca vitrta | 1 | ppm | 100% | 90% | 83% | |
| | 0.1 | ppm | 100% | 23% | 33% | — |
| Phaedon | 10 | ppm | 100% | 100% | — | 100% |
| brassicae | 3 | ppm | 100% | 100% | — | 100% |
| | 1 | ppm | 100% | 100% | — | 100% |
| Thrips tabaci | 10 | ppm | 100% | 86% | — | 90% |
| Lindeman | 3 | ppm | 90% | 83% | — | 76% |
| | 1 | ppm | 70% | 63% | — | 66% |
| Lymantria | 0.1 | ppm | 100% | 95% | 10% | — |
| dispar | 0.01 | ppm | 74% | 5% | 0% | — |
| Grapholita | 10 | ppm | 85% | 85% | 85% | — |
| molesta | | | | | | |
| Rhopobota | 1 | ppm | 100% | 100% | 100% | — |
| naevana | 0.1 | ppm | 100% | 83% | 100% | — |
| Manulea | 1 | ppm | 100% | 16%% | 100% | — |
| degenerella | 0.1 | ppm | 100% | 0% | 100% | — |
| Helicoverpa | 0.1 | ppm | 100% | 78% | 31% | — |

armigera

Referring to Table 1 above, it can be seen that the pesticide compositions according to the present disclosure exhibit an excellent mortality rate against pests such as bugs and moths even if the pesticide compositions include a relatively low concentration of the compound. In particular, the pesticide compositions according to the present disclosure exhibit a significantly high mortality rate against Plutella xylostella, Maruca vitrta, Spodoptera litura, Lymantria dispar, Manulea degenerella, and Helicoverpa armigera even if the pesticide compositions include the compound at a very low concentration of 0.1 ppm. Additionally, it can be seen that the pesticide composition according to the present invention exhibits a high mortality rate even against Thrips tabaci Lindeman and Frankliniella occidentalis.

### Test Example 14—Resistance (Tolerance) Test Against Plutella xylostella

Resistance (tolerance) tests for pesticide compositions were performed on Plutella xylostella. In this case, the Plutella xylostellas used were resistant individuals obtained as follows: radish sprouts were germinated using a moth breeding acrylic cage (25 cm×30 cm×30 cm) to induce egg-laying in a breeding room in the Chungbuk National University (Korea), and the adult insects were collected using an InsectaVac Aspirator (BioQuip®) and induced to lay eggs again by applying a selection pressure using diamide-based chlorantraniliprole at 12.5 ppm once a month to breed for multiple generations.

Cabbage leaves were dipped in the pesticide composition where the compound was diluted (the solvent=distilled water+acetone 50,000 mg/L+Triton X-100 100 mg/L) for 30 seconds, and sufficiently shade-dried. Then, the shade-dried cabbage leaves were placed in a petri dish (8.8 cm in diameter) covered with a filter paper and repeatedly inoculated 3-5 times with third instar larvae of Plutella xylostella, which are resistant individuals, 10 larvae per inoculation. Then, the Plutella xylostella larvae were stored under the conditions of 16 hours of light and 8 hours of dark, 25±1° C., and relative humidity of 50-60%, and the number of live Helicoverpa armigera larvae was counted 24 and 48 hours after the inoculation. Thereafter, the larvae mortality rate was calculated in the same manner as in Test Example 1 above, and the results are shown in Table 2 below.

TABLE 2

| | Concentration of | | | | |
|---|---|---|---|---|---|
| | Compound in | | Example 2 | Comparative | Comparative |
| | Pesticide | | (Synthesis | Example 1 | Example 2 |
| Pests | Composition | | Example 2) | (fluxametamide) | (chlorantraniliprole) |
| Resistant | 1 | ppm | 100% | 58% | 34% |
| Plutella | 0.3 | ppm | 96% | 38% | — |
| xylostella | 0.1 | ppm | 93% | 34% | — |

Referring to Table 2 above, it can be seen that the pesticide compositions according to the present disclosure have almost no resistance (tolerance) and exhibit high mortality rates, whereas those of Comparative Examples 1 and 2 develop resistance thereby showing a significant decrease in the mortality rate.

1. A compound represented by Formula 1 below, a stereoisomer thereof, a hydrate thereof, or a salt thereof: 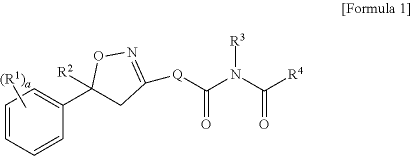
in Formula 1,
R¹ is each independently hydrogen, halogen, cyano (CN), C₁₋₅ alkyl, or C₁₋₅ haloalkyl,
R² is hydrogen, halogen, C₁₋₅ alkyl, or C₁₋₅ haloalkyl,
R³ is hydrogen, C₁₋₅ alkyl, C₃₋₁₀ cycloalkyl, —C(═O)—C₃₋₁₀ cycloalkyl; —C₁₋₅ alkylene-O—C(═O)H substituted with one or more selected from the group consisting of C₁₋₅ alkyl, C₁₋₅ haloalkyl, and C₃₋₁₀ cycloalkyl; or C₁₋₁₀ alkyl substituted with one or more selected from the group consisting of halogen, C₁₋₅ alkyl, C₁₋₅ haloalkyl, and C₃₋₁₀ cycloalkyl,
R⁴ is C₁₋₅ alkyl, C₁₋₅ haloalkyl, C₃₋₁₀ cycloalkyl, C₅₋₁₂ spiroalkyl, 3- to 10-membered heterocycloalkyl, 3- to 10-membered heterocycloalkylene-C(═O)—O—C₁₋₅ alkyl, C₂₋₁₀ alkenyl, C₆₋₂₀ aryl, 3- to 10-membered heteroaryl; C₁₋₅ alkyl substituted with C₃₋₁₀ cycloalkyl; C₃₋₁₀ cycloalkyl substituted with one or more selected from the group consisting of cyano (CN), halogen, C₁₋₅ alkyl, C₁₋₅ haloalkyl, and C₃₋₁₀ cycloalkyl; 3- to 10-membered heterocycloalkyl substituted with one or more selected from the group consisting of halogen, C₁₋₅ alkyl, C₁₋₅ haloalkyl, and C₃₋₁₀ cycloalkyl; C₂₋₁₀ alkenyl substituted with one or more selected from the group consisting of C₁₋₅ alkyl and C₆₋₂₀ aryl; C₆₋₂₀ aryl substituted with one or more selected from the group consisting of cyano (CN), halogen, C₁₋₅ alkyl, and C₁₋₅ haloalkyl; or 3- to 10-membered heteroaryl substituted with one or more selected from the group consisting of halogen, C₁₋₅ alkyl, C₁₋₅ haloalkyl, and C₃₋₁₀ cycloalkyl,
Q is C₆₋₂₀ arylene, 3- to 10-membered heteroarylene, or C₆₋₂₀ arylene substituted with one or more selected from the group consisting of halogen, and C₁₋₅ alkyl,
a is an integer of 1 to 5, and
the heterocycloalkyl, the heteroaryl, the heterocycloalkylene and the heteroarylene each comprise at least one heteroatom selected from the group consisting of N, O, and S.

2. The compound, the stereoisomer thereof, the hydrate thereof, or the salt thereof of claim 1,
wherein
the Q is 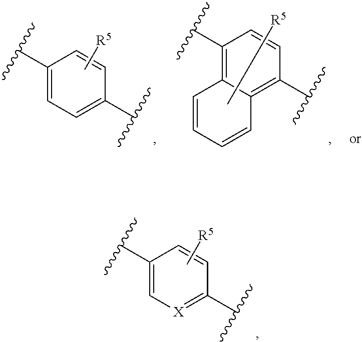
the R⁵ is hydrogen, halogen, or C₁₋₅ alkyl, and
the X is N, O, or S.

3. The compound, the stereoisomer thereof, the hydrate thereof, or the salt thereof of claim 1, wherein Formula 1 above is represented by any one of Formulas 1A to 1E below: 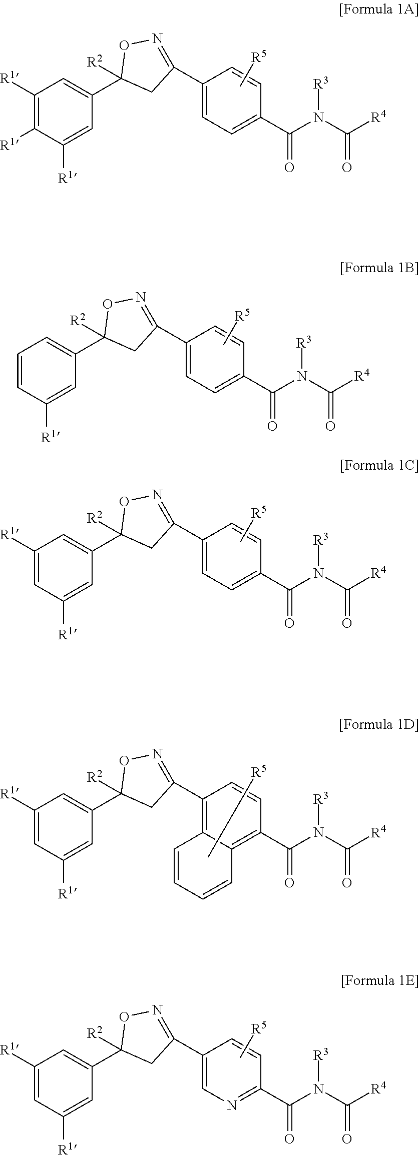
in Formulas 1A to 1E,
R^{1′} is each independently halogen, cyano (CN), C₁₋₅ alkyl, or C₁₋₅ haloalkyl,
the definitions of R² to R⁴ are the same as those defined in claim 1, and
R⁵ is hydrogen, halogen, or C₁₋₅ alkyl.

4. The compound, the stereoisomer thereof, the hydrate thereof, or the salt thereof of claim 3, wherein:
the R^{1′} is each independently cyano (CN), chlorine (Cl), fluorine (F), or C₁₋₃ haloalkyl, and
the R² is C₁₋₃ haloalkyl.

5. The compound, the stereoisomer thereof, the hydrate thereof, or the salt thereof of claim 1, wherein:
the R³ is hydrogen, methyl, ethyl,

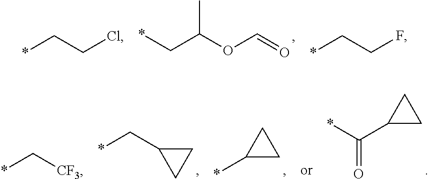

6. The compound, the stereoisomer thereof, the hydrate thereof, or the salt thereof of claim 1, wherein:
the R⁴ is C₃₋₆ cycloalkyl, C₅₋₈ spiroalkyl, 3- to 6-membered heterocycloalkyl, 3- to 6-membered heterocycloalkylene-C(═O)—O—C₁₋₃ alkyl, C₂₋₅ alkenyl, C₆₋₁₀ aryl, 3- to 6-membered heteroaryl; C₁₋₃ alkyl substituted with C₃₋₆ cycloalkyl; C₃₋₆ cycloalkyl substituted with one or more selected from the group consisting of cyano (CN), halogen, and C₁₋₃ alkyl; C₂₋₅ alkenyl substituted with C₆₋₁₀ aryl; C₆₋₁₀ aryl substituted with cyano (CN); or 3- to 6-membered heteroaryl substituted with one or more selected from the group consisting of halogen, C₁₋₃ alkyl, C₁₋₃ haloalkyl, and C₃₋₆ cycloalkyl.

7. The compound, the stereoisomer thereof, the hydrate thereof, or the salt thereof of claim 1, wherein:
the R⁴ is

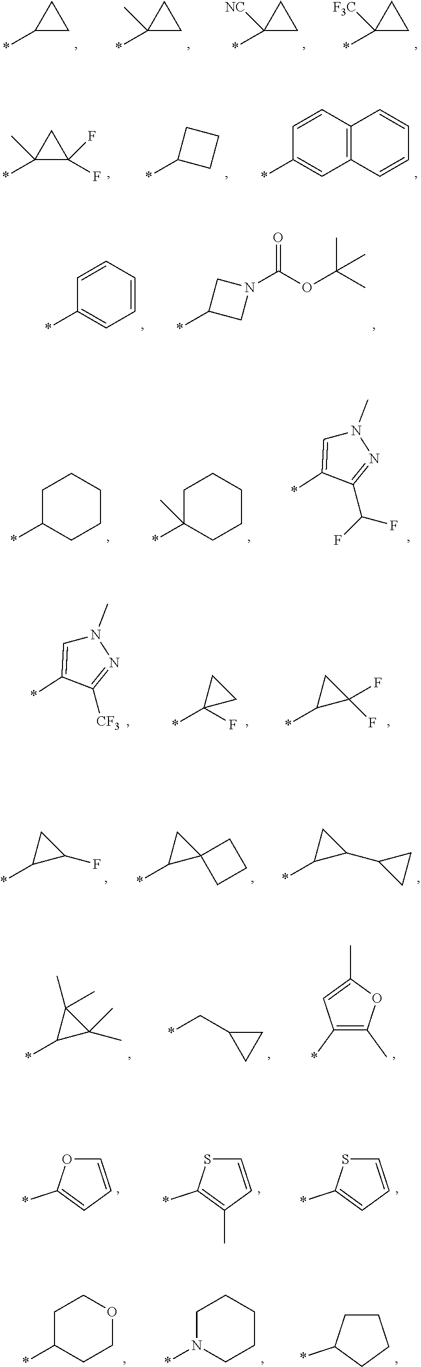

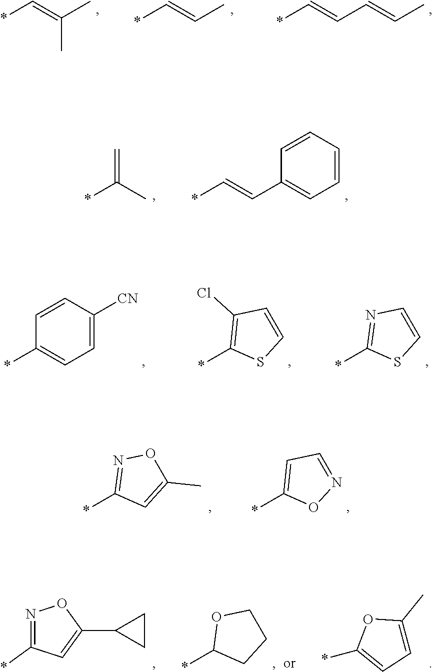

8. The compound, the stereoisomer thereof, the hydrate thereof, or the salt thereof of claim 1, wherein Formula 1 above is represented by any one among Compounds 1001 to 1085 below:

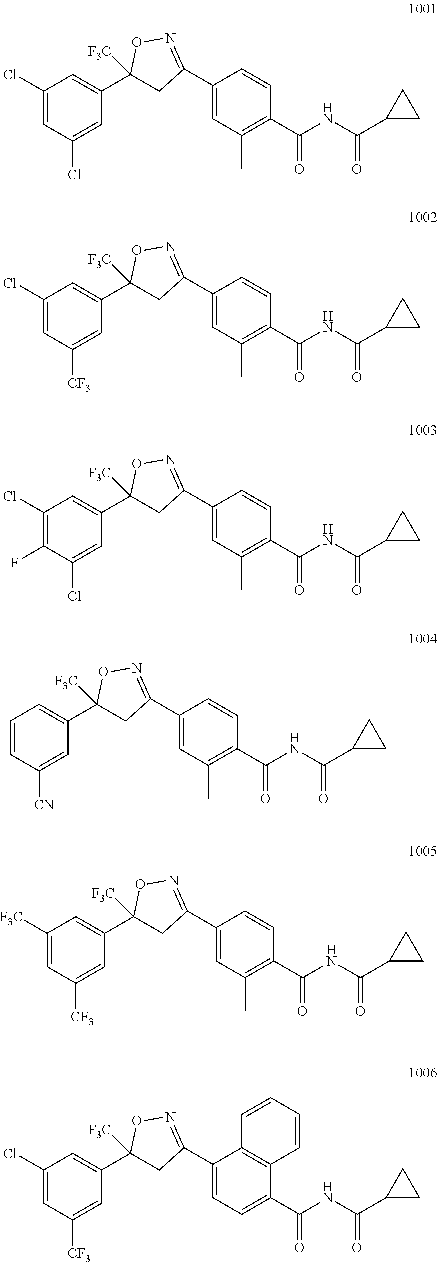 [SMILES: Cc1cc(C2=NOC(c3cc(Cl)cc(Cl)c3)(C(F)(F)F)C2)ccc1C(=O)NC(=O)C1CC1 ; Cc1cc(C2=NOC(c3cc(Cl)cc(C(F)(F)F)c3)(C(F)(F)F)C2)ccc1C(=O)NC(=O)C1CC1 ; Cc1cc(C2=NOC(c3cc(Cl)c(F)c(Cl)c3)(C(F)(F)F)C2)ccc1C(=O)NC(=O)C1CC1 ; Cc1cc(C2=NOC(c3cccc(C#N)c3)(C(F)(F)F)C2)ccc1C(=O)NC(=O)C1CC1 ; Cc1cc(C2=NOC(c3cc(C(F)(F)F)cc(C(F)(F)F)c3)(C(F)(F)F)C2)ccc1C(=O)NC(=O)C1CC1 ; O=C(NC(=O)C1CC1)c1ccc(C2=NOC(c3cc(Cl)cc(C(F)(F)F)c3)(C(F)(F)F)C2)c2ccccc12]

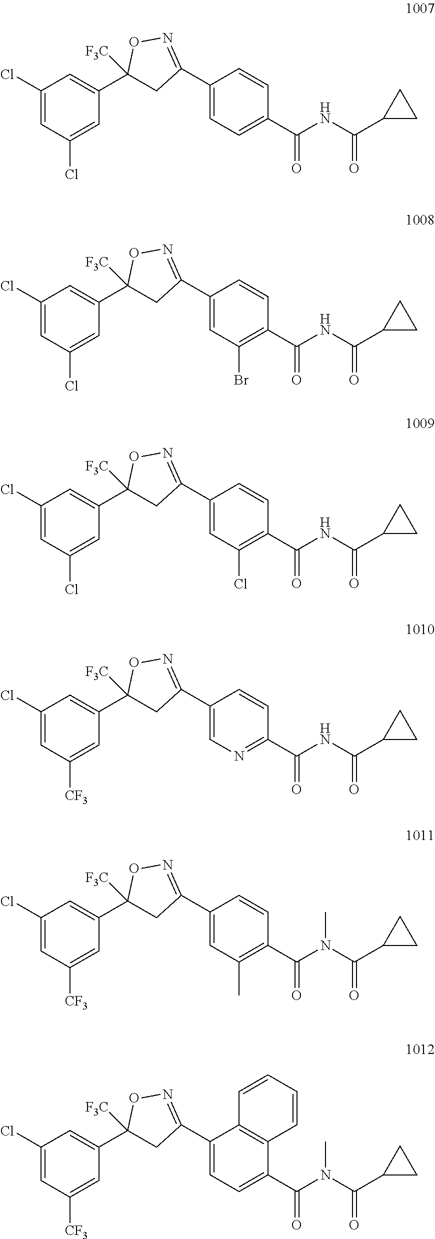 [SMILES: O=C(NC(=O)C1CC1)c1ccc(C2=NOC(c3cc(Cl)cc(Cl)c3)(C(F)(F)F)C2)cc1 ; O=C(NC(=O)C1CC1)c1ccc(C2=NOC(c3cc(Cl)cc(Cl)c3)(C(F)(F)F)C2)cc1Br ; O=C(NC(=O)C1CC1)c1ccc(C2=NOC(c3cc(Cl)cc(Cl)c3)(C(F)(F)F)C2)cc1Cl ; O=C(NC(=O)C1CC1)c1ccc(C2=NOC(c3cc(Cl)cc(C(F)(F)F)c3)(C(F)(F)F)C2)cn1 ; Cc1cc(C2=NOC(c3cc(Cl)cc(C(F)(F)F)c3)(C(F)(F)F)C2)ccc1C(=O)N(C)C(=O)C1CC1 ; CN(C(=O)c1ccc(C2=NOC(c3cc(Cl)cc(C(F)(F)F)c3)(C(F)(F)F)C2)c2ccccc12)C(=O)C1CC1]

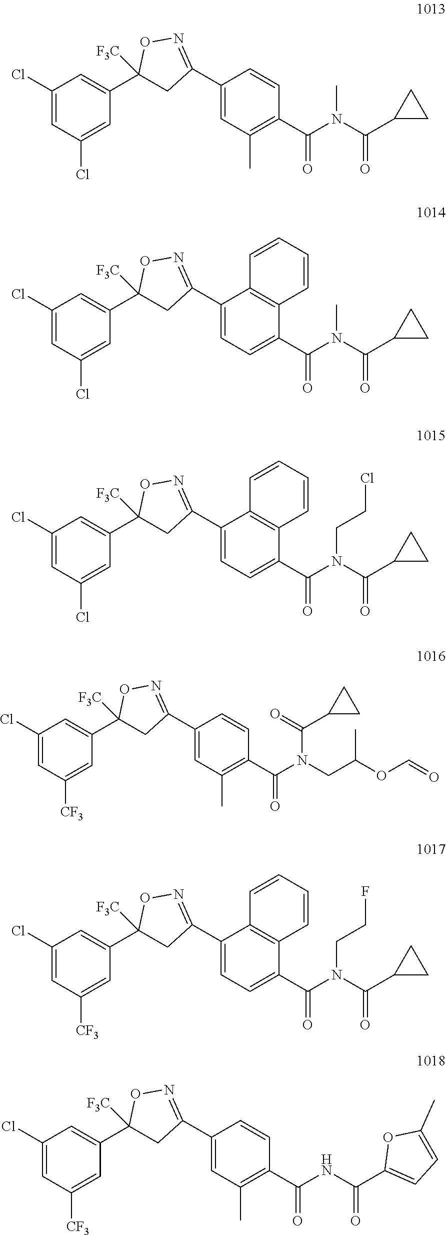 [SMILES: Cc1cc(C2=NOC(c3cc(Cl)cc(Cl)c3)(C(F)(F)F)C2)ccc1C(=O)N(C)C(=O)C1CC1 ; CN(C(=O)c1ccc(C2=NOC(c3cc(Cl)cc(Cl)c3)(C(F)(F)F)C2)c2ccccc12)C(=O)C1CC1 ; O=C(c1ccc(C2=NOC(c3cc(Cl)cc(Cl)c3)(C(F)(F)F)C2)c2ccccc12)N(CCCl)C(=O)C1CC1 ; Cc1cc(C2=NOC(c3cc(Cl)cc(C(F)(F)F)c3)(C(F)(F)F)C2)ccc1C(=O)N(CC(C)OC=O)C(=O)C1CC1 ; O=C(c1ccc(C2=NOC(c3cc(Cl)cc(C(F)(F)F)c3)(C(F)(F)F)C2)c2ccccc12)N(CCF)C(=O)C1CC1 ; Cc1ccc(C(=O)NC(=O)c2ccc(C3=NOC(c4cc(Cl)cc(C(F)(F)F)c4)(C(F)(F)F)C3)cc2C)o1]

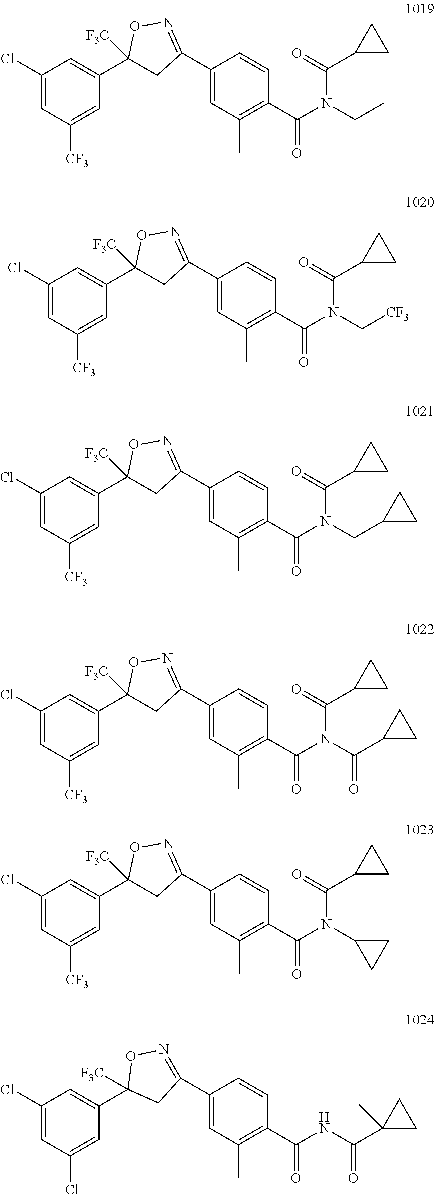 [SMILES: CCN(C(=O)c1ccc(C2=NOC(c3cc(Cl)cc(C(F)(F)F)c3)(C(F)(F)F)C2)cc1C)C(=O)C1CC1 ; Cc1cc(C2=NOC(c3cc(Cl)cc(C(F)(F)F)c3)(C(F)(F)F)C2)ccc1C(=O)N(CC(F)(F)F)C(=O)C1CC1 ; Cc1cc(C2=NOC(c3cc(Cl)cc(C(F)(F)F)c3)(C(F)(F)F)C2)ccc1C(=O)N(CC1CC1)C(=O)C1CC1 ; Cc1cc(C2=NOC(c3cc(Cl)cc(C(F)(F)F)c3)(C(F)(F)F)C2)ccc1C(=O)N(C(=O)C1CC1)C(=O)C1CC1 ; Cc1cc(C2=NOC(c3cc(Cl)cc(C(F)(F)F)c3)(C(F)(F)F)C2)ccc1C(=O)N(C(=O)C1CC1)C1CC1 ; Cc1cc(C2=NOC(c3cc(Cl)cc(Cl)c3)(C(F)(F)F)C2)ccc1C(=O)NC(=O)C1(C)CC1]

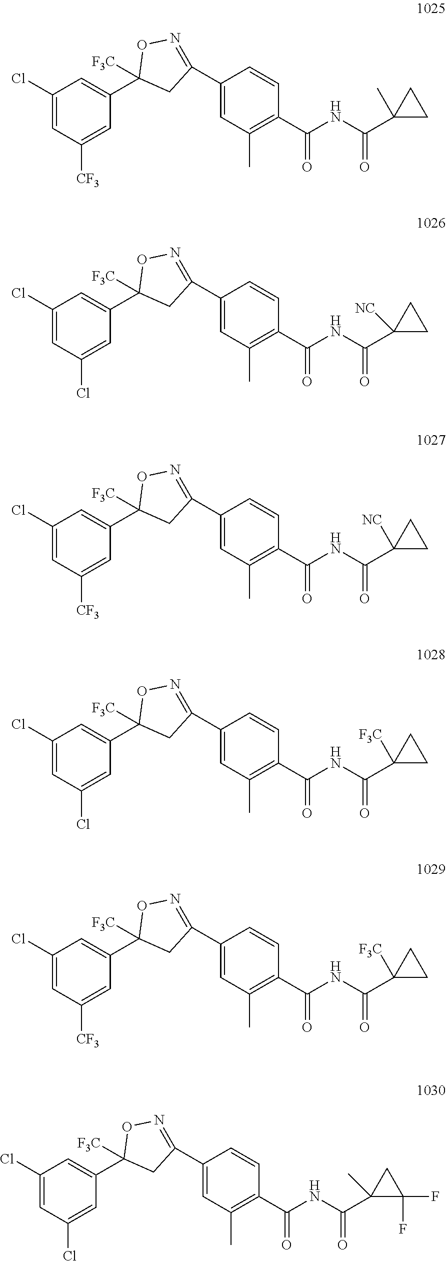 [SMILES: Cc1cc(C2=NOC(c3cc(Cl)cc(C(F)(F)F)c3)(C(F)(F)F)C2)ccc1C(=O)NC(=O)C1(C)CC1 ; Cc1cc(C2=NOC(c3cc(Cl)cc(Cl)c3)(C(F)(F)F)C2)ccc1C(=O)NC(=O)C1(C#N)CC1 ; Cc1cc(C2=NOC(c3cc(Cl)cc(C(F)(F)F)c3)(C(F)(F)F)C2)ccc1C(=O)NC(=O)C1(C#N)CC1 ; Cc1cc(C2=NOC(c3cc(Cl)cc(Cl)c3)(C(F)(F)F)C2)ccc1C(=O)NC(=O)C1(C(F)(F)F)CC1 ; Cc1cc(C2=NOC(c3cc(Cl)cc(C(F)(F)F)c3)(C(F)(F)F)C2)ccc1C(=O)NC(=O)C1(C(F)(F)F)CC1 ; Cc1cc(C2=NOC(c3cc(Cl)cc(Cl)c3)(C(F)(F)F)C2)ccc1C(=O)NC(=O)C1(C)CC1(F)F]

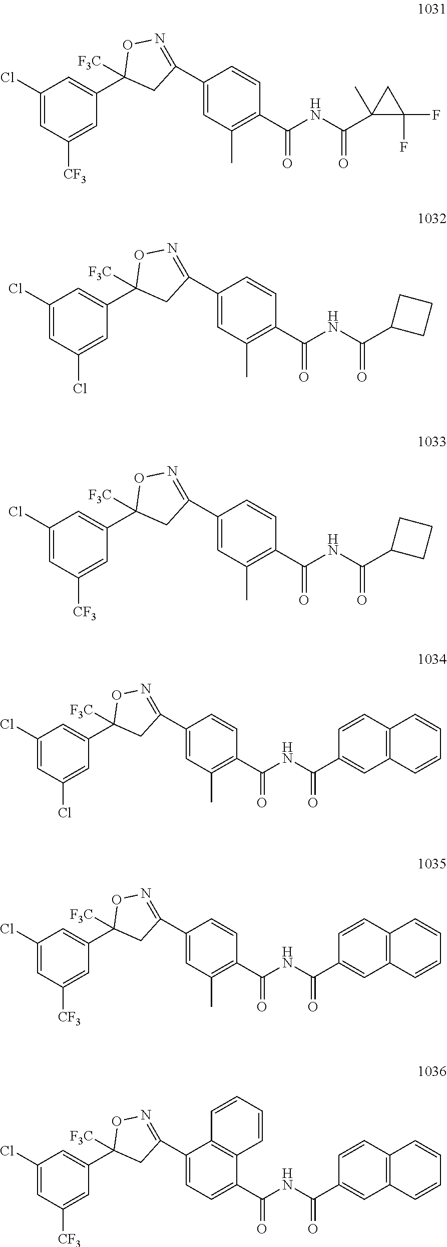 [SMILES: Cc1cc(C2=NOC(c3cc(Cl)cc(C(F)(F)F)c3)(C(F)(F)F)C2)ccc1C(=O)NC(=O)C1(C)CC1(F)F ; Cc1cc(C2=NOC(c3cc(Cl)cc(Cl)c3)(C(F)(F)F)C2)ccc1C(=O)NC(=O)C1CCC1 ; Cc1cc(C2=NOC(c3cc(Cl)cc(C(F)(F)F)c3)(C(F)(F)F)C2)ccc1C(=O)NC(=O)C1CCC1 ; Cc1cc(C2=NOC(c3cc(Cl)cc(Cl)c3)(C(F)(F)F)C2)ccc1C(=O)NC(=O)c1ccc2ccccc2c1 ; Cc1cc(C2=NOC(c3cc(Cl)cc(C(F)(F)F)c3)(C(F)(F)F)C2)ccc1C(=O)NC(=O)c1ccc2ccccc2c1 ; O=C(NC(=O)c1ccc(C2=NOC(c3cc(Cl)cc(C(F)(F)F)c3)(C(F)(F)F)C2)c2ccccc12)c1ccc2ccccc2c1]

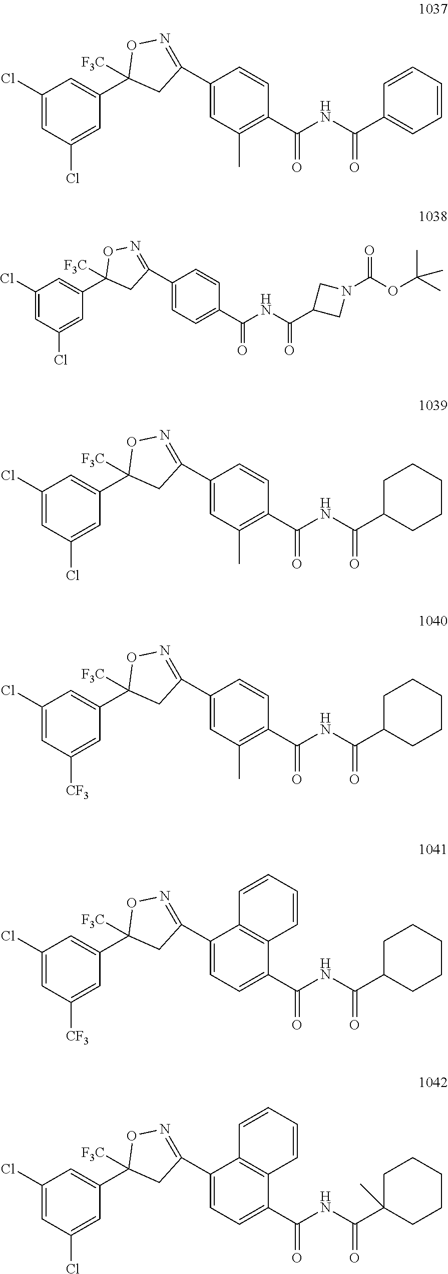 [SMILES: Cc1cc(C2=NOC(c3cc(Cl)cc(Cl)c3)(C(F)(F)F)C2)ccc1C(=O)NC(=O)c1ccccc1 ; CC(C)(C)OC(=O)N1CC(C(=O)NC(=O)c2ccc(C3=NOC(c4cc(Cl)cc(Cl)c4)(C(F)(F)F)C3)cc2)C1 ; Cc1cc(C2=NOC(c3cc(Cl)cc(Cl)c3)(C(F)(F)F)C2)ccc1C(=O)NC(=O)C1CCCCC1 ; Cc1cc(C2=NOC(c3cc(Cl)cc(C(F)(F)F)c3)(C(F)(F)F)C2)ccc1C(=O)NC(=O)C1CCCCC1 ; O=C(NC(=O)C1CCCCC1)c1ccc(C2=NOC(c3cc(Cl)cc(C(F)(F)F)c3)(C(F)(F)F)C2)c2ccccc12 ; CC1(C(=O)NC(=O)c2ccc(C3=NOC(c4cc(Cl)cc(Cl)c4)(C(F)(F)F)C3)c3ccccc23)CCCCC1]

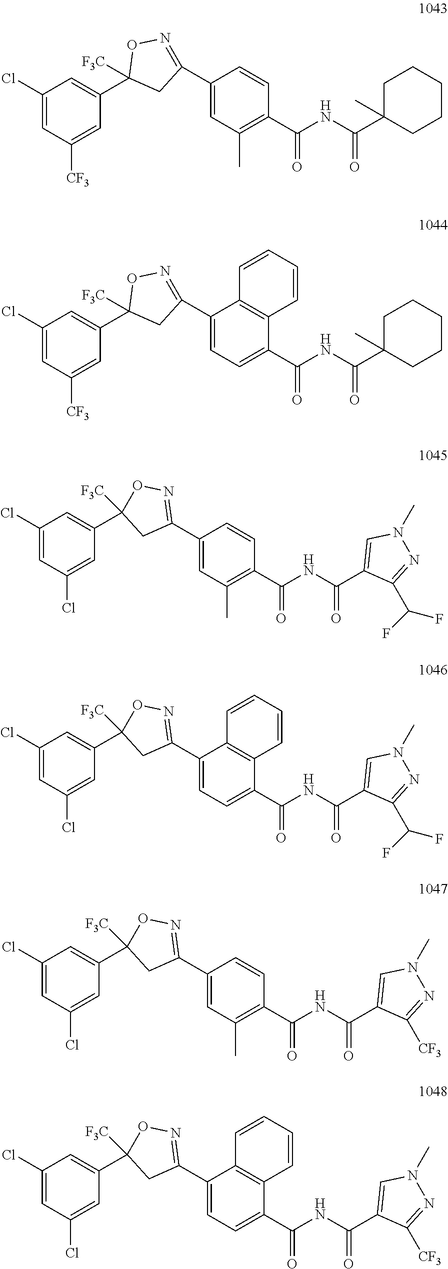 [SMILES: Cc1cc(C2=NOC(c3cc(Cl)cc(C(F)(F)F)c3)(C(F)(F)F)C2)ccc1C(=O)NC(=O)C1(C)CCCCC1 ; CC1(C(=O)NC(=O)c2ccc(C3=NOC(c4cc(Cl)cc(C(F)(F)F)c4)(C(F)(F)F)C3)c3ccccc23)CCCCC1 ; Cc1cc(C2=NOC(c3cc(Cl)cc(Cl)c3)(C(F)(F)F)C2)ccc1C(=O)NC(=O)c1cn(C)nc1C(F)F ; Cn1cc(C(=O)NC(=O)c2ccc(C3=NOC(c4cc(Cl)cc(Cl)c4)(C(F)(F)F)C3)c3ccccc23)c(C(F)F)n1 ; Cc1cc(C2=NOC(c3cc(Cl)cc(Cl)c3)(C(F)(F)F)C2)ccc1C(=O)NC(=O)c1cn(C)nc1C(F)(F)F ; Cn1cc(C(=O)NC(=O)c2ccc(C3=NOC(c4cc(Cl)cc(Cl)c4)(C(F)(F)F)C3)c3ccccc23)c(C(F)(F)F)n1]

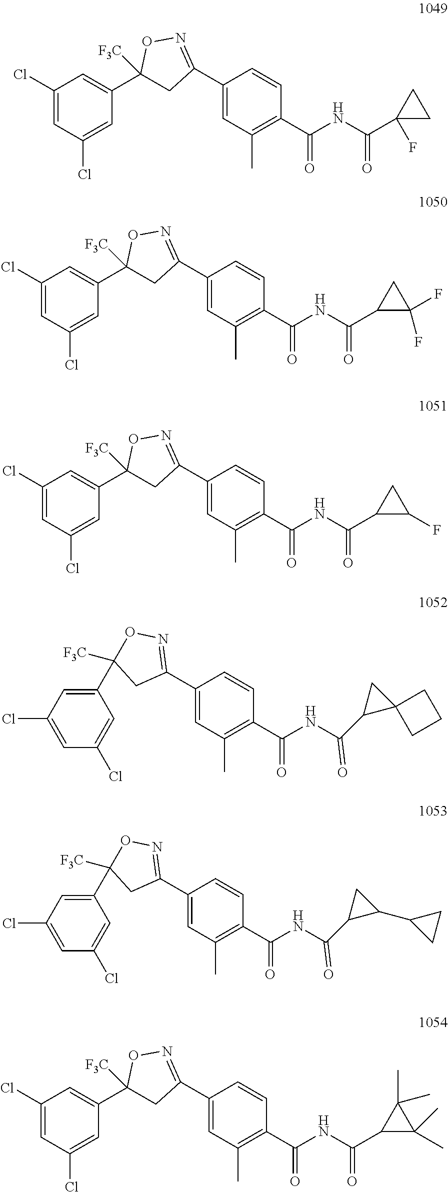 [SMILES: Cc1cc(C2=NOC(c3cc(Cl)cc(Cl)c3)(C(F)(F)F)C2)ccc1C(=O)NC(=O)C1(F)CC1 ; Cc1cc(C2=NOC(c3cc(Cl)cc(Cl)c3)(C(F)(F)F)C2)ccc1C(=O)NC(=O)C1CC1(F)F ; Cc1cc(C2=NOC(c3cc(Cl)cc(Cl)c3)(C(F)(F)F)C2)ccc1C(=O)NC(=O)C1CC1F ; Cc1cc(C2=NOC(c3cc(Cl)cc(Cl)c3)(C(F)(F)F)C2)ccc1C(=O)NC(=O)C1CC12CCC2 ; Cc1cc(C2=NOC(c3cc(Cl)cc(Cl)c3)(C(F)(F)F)C2)ccc1C(=O)NC(=O)C1CC1C1CC1 ; Cc1cc(C2=NOC(c3cc(Cl)cc(Cl)c3)(C(F)(F)F)C2)ccc1C(=O)NC(=O)C1C(C)(C)C1(C)C]

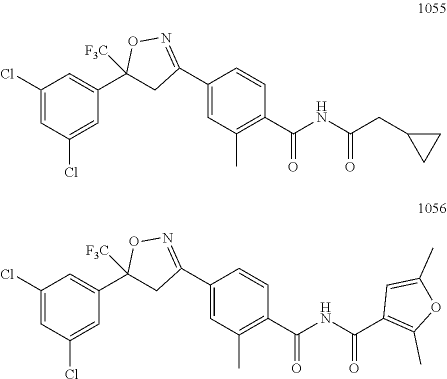 [SMILES: Cc1cc(C2=NOC(c3cc(Cl)cc(Cl)c3)(C(F)(F)F)C2)ccc1C(=O)NC(=O)CC1CC1 ; Cc1cc(C(=O)NC(=O)c2ccc(C3=NOC(c4cc(Cl)cc(Cl)c4)(C(F)(F)F)C3)cc2C)c(C)o1]

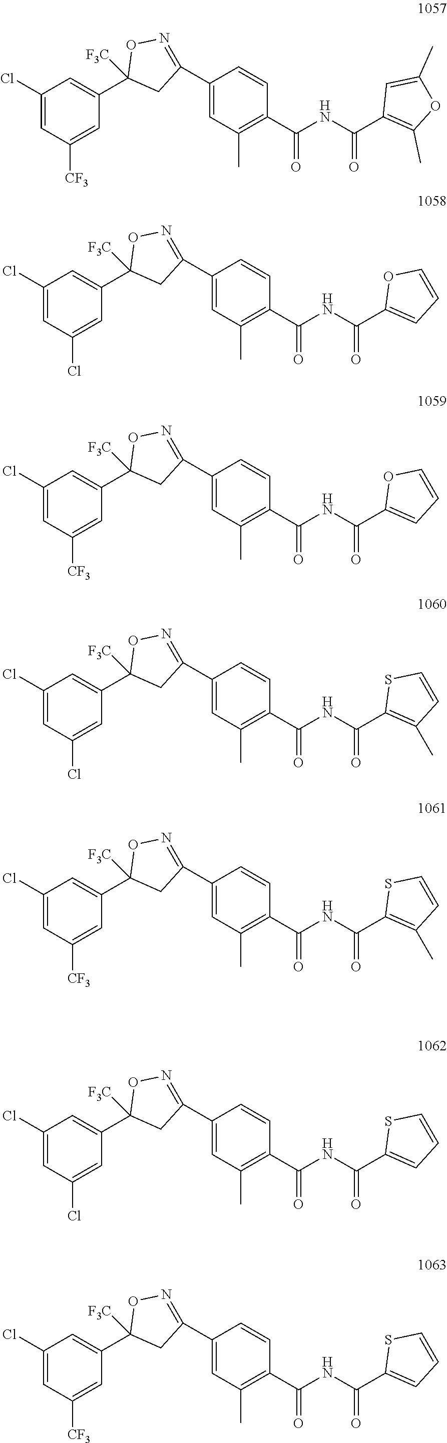

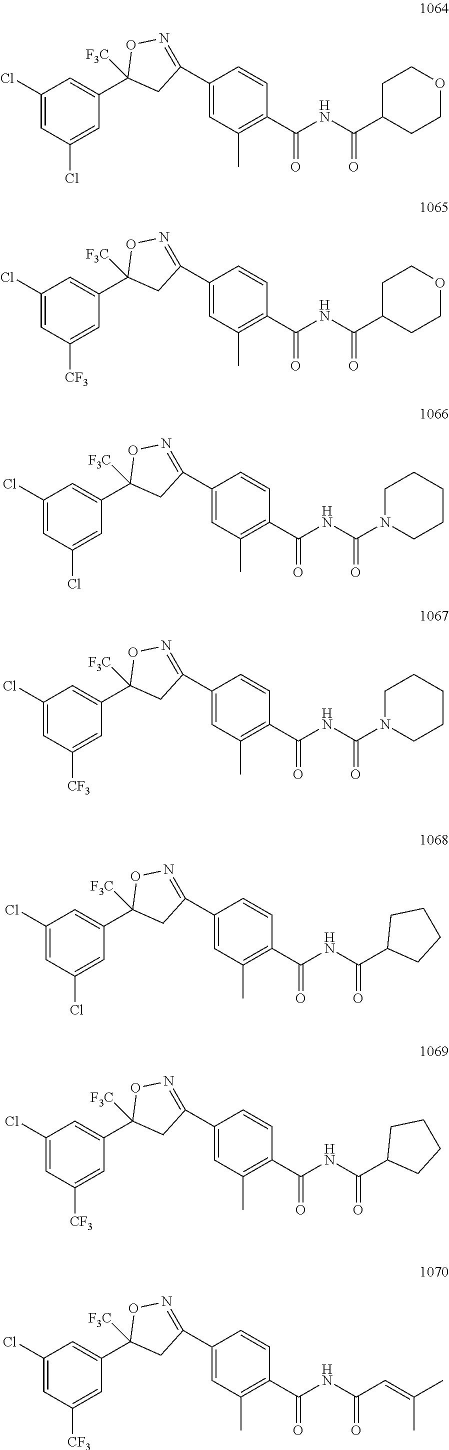

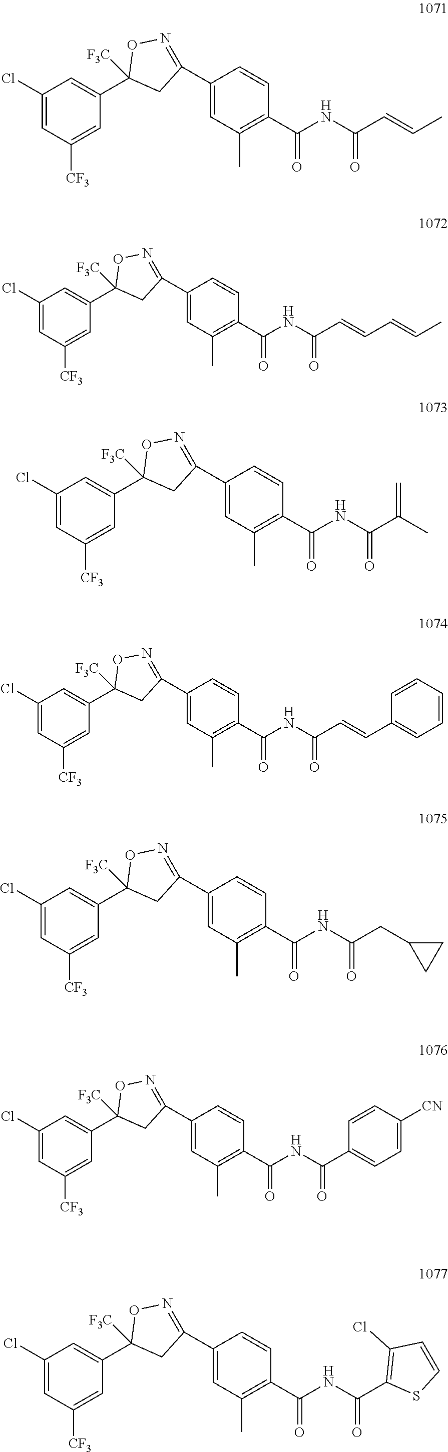

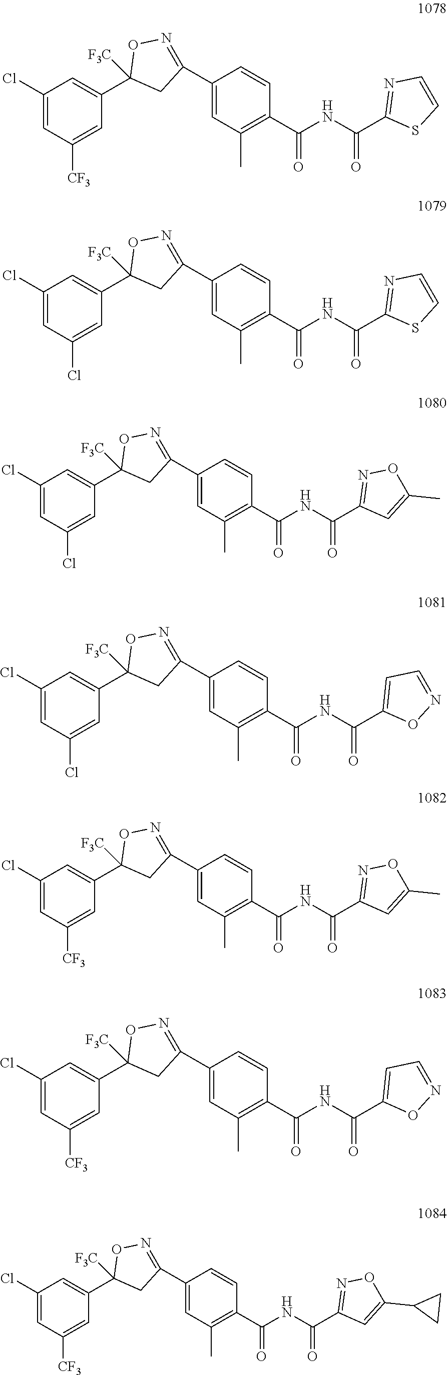

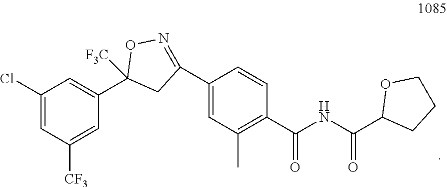 [SMILES: Cc1cc(C2=NOC(c3cc(Cl)cc(C(F)(F)F)c3)(C(F)(F)F)C2)ccc1C(=O)NC(=O)C1CCCO1]

9. A pesticide composition comprising, as an active ingredient, one or more compounds selected from the group consisting of the compound, the stereoisomer thereof, the hydrate thereof, and the salt thereof of claim 1.

10. The pesticide composition of claim 9, wherein the composition is for controlling pests of the order Thysanoptera or Lepidoptera.

11. The pesticide composition of claim 9, wherein the composition is for controlling Frankliniella occidentalis, Frankliniella tenuicornis, Frankliniella intonsa, Frankliniella lilivora, Thrips palmi Karny, Thrips tabaci Lindeman, Phaedon brassicae, Myzus persicae, Riptortus clavatus, Lymantria dispar, Helicoverpa armigera, Manulea degenerella, Rhopobota naevana, Grapholita molesta, Plutella xylostella, Spodoptera litura, Spodoptera exigua, Spodoptera frugiperda, or Maruca vitrta.

12. A method for controlling pests, comprising applying the pesticide composition of claim 9 to crops or their habitats.

13. The method of claim 12, wherein the pests belong to pests of order Thysanoptera or Lepidoptera.

14. The method of claim 12, wherein the pests are Frankliniella occidentalis, Frankliniella tenuicornis, Frankliniella intonsa, Frankliniella lilivora, Thrips palmi Karny, Thrips tabaci Lindeman, Phaedon brassicae, Myzus persicae, Riptortus clavatus, Lymantria dispar, Helicoverpa armigera, Manulea degenerella, Rhopobota naevana, Grapholita molesta, Plutella
